SEQUENCE LISTING (20 sequences)

SEQ ID NO: 1 - DNA, 573 bp, Prototheca moriformis
tgttgaagaatgagccggcgacttaaaataaatggcaggctaagagaattaataactcgaaacctaagcgaaagcaagtcttaatagggcgctaatttaacaaaacattaaataaaatctaaagtcatttattttagacccgaacctgagtgatctaaccatggtcaggatgaaacttgggtgacaccaagtggaagtccgaaccgaccgatgttgaaaaatcggcggatgaactgtggttagtggtgaaataccagtcgaactcagagctagctggttctccccgaaatgcgttgaggcgcagcaatatatctcgtctatctaggggtaaagcactgtttcggtgcgggctatgaaaatggtaccaaatcgtggcaaactctgaatactagaaatgacgatatattagtgagactatgggggataagctccatagtcgagagggaaacagcccagaccaccagttaaggccccaaaatgataatgaagtggtaaaggaggtgaaaatgcaaatacaaccaggaggttggcttagaagcagccatcctttaaagagtgcgtaatagctcactg

SEQ ID NO: 2 - protein, 431 aa, Protheca moriformis
AAAAADANPARPERRVVITGQGVVTSLGQTIEQFYSSLLEGVSGISQIQKFDTTGYTTTIAGEIKSLQLDPYVPKRWAKRVDDVIKYVYIAGKQALESAGLPIEAAGLAGAGLDPALCGVLIGTAMAGMTSFAAGVEALTRGGVRKMNPFCIPFSISNMGGAMLAMDIGFMGPNYSISTACATGNYCILGAADHIRRGDANVMLAGGADAAIIPSGIGGFIACKALSKRNDEPERASRPWDADRDGFVMGEGAGVLVLEELEHAKRRGATILAELVGGAATSDAHHMTEPDPQGRGVRLCLERALERARLAPERVGYVNAHGTSTPAGDVAEYRAIRAVIPQDSLRINSTKSMIGHLLGGAGAVEAVAAIQALRTGWLHPNLNLENPAPGVDPVVLVGPRKERAEDLDVVLSNSFGFGGHNSCVIFRKYDE

SEQ ID NO: 3 - DNA, 1191 bp, Artificial Sequence
  source: /note=“Description of Artificial Sequence Synthetic polynucleotide”
actagtatgctgaagctgtcctgcaacgtgaccaacaacctgcacaccttctccttcttctccgactcctccctgttcatccccgtgaaccgccgcaccatcgccgtgtcctccgggcgcgcctcccagctgcgcaagcccgccctggaccccctgcgcgccgtgatctccgccgaccagggctccatctcccccgtgaactcctgcacccccgccgaccgcctgcgcgccggccgcctgatggaggacggctactcctacaaggagaagttcatcgtgcgctcctacgaggtgggcatcaacaagaccgccaccgtggagaccatcgccaacctgctgcaggaggtggcctgcaaccacgtgcagaagtgcggcttctccaccgacggcttcgccaccaccctgaccatgcgcaagctgcacctgatctgggtgaccgcccgcatgcacatcgagatctacaagtaccccgcctggtccgacgtggtggagatcgagacctggtgccagtccgagggccgcatcggcacccgccgcgactggatcctgcgcgactccgccaccaacgaggtgatcggccgcgccacctccaagtgggtgatgatgaaccaggacacccgccgcctgcagcgcgtgaccgacgaggtgcgcgacgagtacctggtgttctgcccccgcgagccccgcctggccttccccgaggagaacaactcctccctgaagaagatccccaagctggaggaccccgcccagtactccatgctggagctgaagccccgccgcgccgacctggacatgaaccagcacgtgaacaacgtgacctacatcggctgggtgctggagtccatcccccaggagatcatcgacacccacgagctgcaggtgatcaccctggactaccgccgcgagtgccagcaggacgacatcgtggactccctgaccacctccgagatccccgacgaccccatctccaagttcaccggcaccaacggctccgccatgtcctccatccagggccacaacgagtcccagttcctgcacatgctgcgcctgtccgagaacggccaggagatcaaccgcggccgcacccagtggcgcaagaagtcctcccgcatggactacaaggaccacgacggcgactacaaggaccacgacatcgactacaaggacgacgacgacaagtgaatcgat

SEQ ID NO: 4 - protein, 392 aa, Artificial Sequence
  source: /note=“Description of Artificial Sequence Synthetic polynucleotide”
MLKLSCNVTNNLHTFSFFSDSSLFIPVNRRTIAVSSGRASQLRKPALDPLRAVISADQGSISPVNSCTPADRLRAGRLMEDGYSYKEKFIVRSYEVGINKTATVETIANLLQEVACNHVQKCGFSTDGFATTLTMRKLHLIWVTARMHIEIYKYPAWSDVVEIETWCQSEGRIGTRRDWILRDSATNEVIGRATSKWVMMNQDTRRLQRVTDEVRDEYLVFCPREPRLAFPEENNSSLKKIPKLEDPAQYSMLELKPRRADLDMNQHVNNVTYIGWVLESIPQEIIDTHELQVITLDYRRECQQDDIVDSLTTSEIPDDPISKFTGTNGSAMSSIQGHNESQFLHMLRLSENGQEINRGRTQWRKKSSRMDYKDHDGDYKDHDIDYKDDDDK

SEQ ID NO: 5 - protein, 391 aa, Artificial Sequence
  source: /note=“Description of Artificial Sequence Synthetic polypeptide”
MATASTFSAFNARCGDLRRSAGSGPRRPARPLPVRGRASQLRKPALDPLRAVISADQGSISPVNSCTPADRLRAGRLMEDGYSYKEKFIVRSYEVGINKTATVETIANLLQEVACNHVQKCGFSTDGFATTLTMRKLHLIWVTARMHIEIYKYPAWSDVVEIETWCQSEGRIGTRRDWILRDSATNEVIGRATSKWVMMNQDTRRLQRVTDEVRDEYLVFCPREPRLAFPEENNSSLKKIPKLEDPAQYSMLELKPRRADLDMNQHVNNVTYIGWVLESIPQEIIDTHELQVITLDYRRECQQDDIVDSLTTSEIPDDPISKFTGTNGSAMSSIQGHNESQFLHMLRLSENGQEINRGRTQWRKKSSRMDYKDHDGDYKDHDIDYKDDDDK

SEQ ID NO: 6 - protein, 384 aa, Artificial Sequence
  source: /note=“Description of Artificial Sequence Synthetic polypeptide”
MATASTFSAFNARCGDLRRSAGSGPRRPARPLPVRGRAATGEQPSGVASLREADKEKSLGNRLRLGSLTEDGLSYKEKFVIRCYEVGINKTATIETIANLLQEVGGNHAQGVGFSTDGFATTTTMRKLHLIWVTARMHIEIYRYPAWSDVIEIETWVQGEGKVGTRRDWILKDYANGEVIGRATSKWVMMNEDTRRLQKVSDDVREEYLVFCPRTLRLAFPEENNNSMKKIPKLEDPAEYSRLGLVPRRSDLDMNKHVNNVTYIGWALESIPPEIIDTHELQAITLDYRRECQRDDIVDSLTSREPLGNAAGVKFKEINGSVSPKKDEQDLSRFMHLLRSAGSGLEINRCRTEWRKKPAKRMDYKDHDGDYKDHDIDYKDDDDK

SEQ ID NO: 7 - protein, 397 aa, Artificial Sequence
  source: /note=“Description of Artificial Sequence Synthetic polypeptide”
MLKVPCCNATDPIQSLSSQCRFLTHFNNRPYFTRRPSIPTFFSSKNSSASLQAVVSDISSVESAACDSLANRLRLGKLTEDGFSYKEKFIVGRARSYEVGINKTATVETIANLLQEVGCNHAQSVGFSTDGFATTTSMRKMHLIWVTARMHIEIYKYPAWSDVVEVETWCQSEGRIGTRRDWILTDYATGQIIGRATSKWVMMNQDTRRLQKVTDDVREEYLVFCPRELRLAFPEENNRSSKKISKLEDPAQYSKLGLVPRRADLDMNQHVNNVTYIGWVLESIPQEIIDTHELQTITLDYRRECQHDDIVDSLTSVEPSENLEAVSELRGTNGSATTTAGDEDCRNFLHLLRLSGDGLEINRGRTEWRKKSARMDYKDHDGDYKDHDIDYKDDDDK

SEQ ID NO: 8 - protein, 398 aa, Artificial Sequence
  source: /note=“Description of Artificial Sequence Synthetic polypeptide”
MLKLSSCNVTDQRQALAQCRFLAPPAPFSFRWRTPVVVSCSPSSRPNLSPLQVVLSGQQQAGMELVESGSGSLADRLRLGSLTEDGLSYKEKFIVRCYEVGINKTATVETIANLLQEVGCNHAQSVGYSTDGFATTRTMRKLHLIWVTARMHIEIYKYPAWSDVIEIETWCQSEGRIGTRRDWILKDFGTGEVIGRATSKWVMMNQDTRRLQKVSDDVREEYLVFCPRELRLAFPEENNNSLKKIAKLDDSFQYSRLGLMPRRADLDMNQHVNNVTYIGWVLESMPQEIIDTHELQTITLDYRRECQQDDVVDSLTSPEQVEGTEKVSAIHGTNGSAAAREDKQDCRQFLHLLRLSSDGQEINRGRTEWRKKPARMDYKDHDGDYKDHDIDYKDDDDK

SEQ ID NO: 9 - protein, 375 aa, Artificial Sequence
  source: /note=“Description of Artificial Sequence Synthetic polypeptide”
MLKLSSSRSPLARIPTRPRPNSIPPRIIVVSSSSSKVNPLKTEAVVSSGLADRLRLGSLTEDGLSYKEKFIVRCYEVGINKTATVETIANLLQEVGCNHAQSVGYSTGGFSTTPTMRKLRLIWVTARMHIEIYKYPAWSDVVEIESWGQGEGKIGTRRDWILRDYATGQVIGRATSKWVMMNQDTRRLQKVDVDVRDEYLVHCPRELRLAFPEENNSSLKKISKLEDPSQYSKLGLVPRRADLDMNQHVNNVTYIGWVLESMPQEIIDTHELQTITLDYRRECQHDDVVDSLTSPEPSEDAEAVFNHNGTNGSANVSANDHGCRNFLHLLRLSGNGLEINRGRTEWRKKPTRMDYKDHDGDYKDHDIDYKDDDDK

SEQ ID NO: 10 - protein, 452 aa, Prototheca moriformis
MSIQFALRAAYIKGTCQRLSGRGAALGLSRDWTPGWTLPRCWPASAAATAPPRARHQERAIHLTSGRRRHSALASDADERALPSNAPGLVMASQANYFRVRLLPEQEEGELESWSPNVRHTTLLCKPRAMLSKLQMRVMVGDRVIVTAIDPVNMTVHAPPFDPLPATRFLVAGEAADMDITVVLNKADLVPEEESAALAQEVASWGPVVLTSTLTGRGLQELERQLGSTTAVLAGPSGAGKSSIINALARAARERPSDASVSNVPEEQVVGEDGRALANPPPFTLADIRNAIPKDCFRKSAAKSLAYLGDLSITGMAVLAYKINSPWLWPLYWFAQGTMFWALFVVGHDCGHQSFSTSKRLNDALAWLGALAAGTWTWALGVLPMLNLYLAPYVWLLVTYLHHHGPSDPREEMPWYRGREWSYMRGGLTTIDRDYGLFNKVHHDIGTHVVHH

SEQ ID NO: 11 - protein, 315 aa, Prototheca moriformis
MFWALFVVGHDCGHQSFSTSKRLNDAVGLFVHSIIGVPYHGWRISHRTHHNNHGHVENDESWYPPTESGLKAMTDMGRQGRFHFPSMLFVYPFYLFWRSPGKTGSHFSPATDLFALWEAPLIRTSNACQLAWLGALAAGTWALGVLPMLNLYLAPYVISVAWLDLVTYLHHHGPSDPREEMPWYRGREWSYMRGGLTTIDRDYGLFNKVHHDIGTHVVHHLFPQIPHYNLCRATKAAKKVLGPYYREPERCPLGLLPVHLLAPLLRSLGQDHFVDDAGSVLFYRRAEGINPWIQKLLPWLGGARRGADAQRDAAQ

SEQ ID NO: 12 - protein, 448 aa, Camelina sativa
MANLVLSECGIRPLPRIYTTPRSNFVSNNNKPIFKFRPFTSYKTSSSPLACSRDGFGKNWSLNVSVPLTTTTPIVDESPLKEEEEEKQRFDPGAPPPFNLADIRAAIPKHCWVKNPWKSMSYVLRDVAIVFALAAGASYLNNWIVWPLYWLAQGTMFWALFVLGHDCGHGSFSNNPRLNNVVGHLLHSSILVPYHGWRISHRTHHQNHGHVENDESWHPMSEKIYQSLDKPTRFFRFTLPLVMLAYPFYLWARSPGKKGSHYHPESDLFLPKEKTDVLTSTACWTAMAALLICLNFVVGPVQMLKLYGIPYWINVMWLDFVTYLHHHGHEDKLPWYRGKEWSYLRGGLTTLDRDYGVINNIHHDIGTHVIHHLFPQIPHYHLVEATEAVKPVLGKYYREPDKSGPLPLHLLGILAKSIKEDHYVSDEGDVVYYKADPNMYGEIKVGAD

SEQ ID NO: 13 - protein, 368 aa, Prototheca moriformis
MAIKTNRQPVEKPPFTIGTLRKAIPAHCFERSALRSSMYLAFDIAVMSLLYVASTYIDPAPVPTWVKYGIMWPLYWFFQGAFGTGVWVCAHECGHQAFSSSQAINDGVGLVFHSLLLVPYYSWKHSHRRHHSNTGCLDKDEVFVPPHRAVAHEGLEWEEWLPIRMGKVLVTLTLGWPLYLMFNVASRPYPRFANHFDPWSPIFSKRERIEVVISDLALVAVLSGLSVLGRTMGWAWLVKTYVVPYMIVNMWLVLITLLQHTHPALPHYFEKDWDWLRGAMATVDRSMGPPFMDSILHHISDTHVLHHLFSTIPHYHAEEASAAIRPILGKYYQSDSRWVGRALWEDWRDCRYVVPDAPEDDSALWFHK

SEQ ID NO: 14 - protein, 448 aa, Camelina sativa
MANLVLSECGIRPLPRIYTTPRSNFVSNNNKPIFKFRPLTSYKTSSPLFCSRDGFGRNWSLNVSVPLATTTPIVDESPLEEEEEEEKQRFDPGAPPPFNLADIRAAIPKHCWVKNPWKSMSYVLRDVAIVFALAAGAAYLNNWIVWPLYWLAQGTMFWALFVLGHDCGHGSFSNNPRLNNVVGHLLHSSILVPYHGWRISHRTHHQNHGHVENDESWHPMSEKIYQSLDKPTRFFRFTLPLVMLAYPFYLWARSPGKKGSHYHPESDLFLPKEKTDVLTSTACWTAMAALLICLNFVVGPVQMLKLYGIPYWINVMWLDFVTYLHHHGHEDKLPWYRGKEWSYLRGGLTTLDRDYGVINNIHHDIGTHVIHHLFPQIPHYHLVEATEAVKPVLGKYYREPDKSGPLPLHLLGILAKSIKEDHYVSDEGDVVYYKADPNMYGEIKVGAD

SEQ ID NO: 15 - DNA, 10521 bp, Artificial Sequence
  source: /note=“Description of Artificial Sequence Synthetic polynucleotide”
agcccgcaccctcgttgatctgggagccctgcgcagccccttaaatcatctcagtcaggtttctgtgttcaactgagcctaaagggctttcgtcatgcgcacgagcacacgtatatcggccacgcagtttctcaaaagcggtagaacagttcgcgagccctcgtaggtcgaaaacttgcgccagtactattaaattaaattaattgatcgaacgagacgcgaaacttttgcagaatgccaccgagtttgcccagagaatgggagtggcgccattcaccatccgcctgtgcccggcttgattcgccgagacgatggacggcgagaccagggagcggcttgcgagccccgagccggtagcaggaacaatgatcgacaatcttcctgtccaattactggcaaccattagaaagagccggagcgcgttgaaagtctgcaatcgagtaatttttcgatacgtcgggcctgctgaaccctaaggctccggactttgtttaaggcgatccaagatgcacgcggccccaggcacgtatctcaagcacaaaccccagccttagtttcgagactttgggagatagcgaccgatatctagtttggcattttgtatattaattacctcaagcaatggagcgctctgatgcggtgcagcgtcggctgcagcacctggcagtggcgctagggtcgccctatcgctcggaacctggtcagctggctcccgcctcctgctcagcctcttccggtaccctttcttgcgctatgacacttccagcaaaaggtagggcgggctgcgagacggcttcccggcgctgcatgcaacaccgatgatgcttcgaccccccgaagctccttcggggctgcatgggcgctccgatgccgctccagggcgagcgctgtttaaatagccaggcccccgattgcaaagacattatagcgagctaccaaagccatattcaaacacctagatcactaccacttctacacaggccactcgagcttgtgatcgcactccgctaagggggcgcctcttcctcttcgtttcagtcacaacccgcaaactctagaatatcaatgctgctgcaggccttcctgttcctgctggccggcttcgccgccaagatcagcgcctccatgacgaacgagacgtccgaccgccccctggtgcacttcacccccaacaagggctggatgaacgaccccaacggcctgtggtacgacgagaaggacgccaagtggcacctgtacttccagtacaacccgaacgacaccgtctgggggacgcccttgttctggggccacgccacgtccgacgacctgaccaactgggaggaccagcccatcgccatcgccccgaagcgcaacgactccggcgccttctccggctccatggtggtggactacaacaacacctccggcttcttcaacgacaccatcgacccgcgccagcgctgcgtggccatctggacctacaacaccccggagtccgaggagcagtacatctcctacagcctggacggcggctacaccttcaccgagtaccagaagaaccccgtgctggccgccaactccacccagttccgcgacccgaaggtcttctggtacgagccctcccagaagtggatcatgaccgcggccaagtcccaggactacaagatcgagatctactcctccgacgacctgaagtcctggaagctggagtccgcgttcgccaacgagggcttcctcggctaccagtacgagtgccccggcctgatcgaggtccccaccgagcaggaccccagcaagtcctactgggtgatgttcatctccatcaaccccggcgccccggccggcggctccttcaaccagtacttcgtcggcagcttcaacggcacccacttcgaggccttcgacaaccagtcccgcgtggtggacttcggcaaggactactacgccctgcagaccttcttcaacaccgacccgacctacgggagcgccctgggcatcgcgtgggcctccaactgggagtactccgccttcgtgcccaccaacccctggcgctcctccatgtccctcgtgcgcaagttctccctcaacaccgagtaccaggccaacccggagacggagctgatcaacctgaaggccgagccgatcctgaacatcagcaacgccggcccctggagccggttcgccaccaacaccacgttgacgaaggccaacagctacaacgtcgacctgtccaacagcaccggcaccctggagttcgagctggtgtacgccgtcaacaccacccagacgatctccaagtccgtgttcgcggacctctccctctggttcaagggcctggaggaccccgaggagtacctccgcatgggcttcgaggtgtccgcgtcctccttcttcctggaccgcgggaacagcaaggtgaagttcgtgaaggagaacccctacttcaccaaccgcatgagcgtgaacaaccagcccttcaagagcgagaacgacctgtcctactacaaggtgtacggcttgctggaccagaacatcctggagctgtacttcaacgacggcgacgtcgtgtccaccaacacctacttcatgaccaccgggaacgccctgggctccgtgaacatgacgacgggggtggacaacctgttctacatcgacaagttccaggtgcgcgaggtcaagtgacaattgacgcccgcgcggcgcacctgacctgttctctcgagggcgcctgttctgccttgcgaaacaagcccctggagcatgcgtgcatgatcgtctctggcgccccgccgcgcggtttgtcgccctcgcgggcgccgcggccgcgggggcgcattgaaattgttgcaaaccccacctgacagattgagggcccaggcaggaaggcgttgagatggaggtacaggagtcaagtaactgaaagtttttatgataactaacaacaaagggtcgtttctggccagcgaatgacaagaacaagattccacatttccgtgtagaggcttgccatcgaatgtgagcgggcgggccgcggacccgacaaaacccttacgacgtggtaagaaaaacgtggcgggcactgtccctgtagcctgaagaccagcaggagacgatcggaagcatcacagcacaggatcccgcgtctcgaacagagcgcgcagaggaacgctgaaggtctcgcctctgtcgcacctcagcgcggcatacaccacaataaccacctgacgaatgcgcttggttcttcgtccattagcgaagcgtccggttcacacacgtgccacgttggcgaggtggcaggtgacaatgatcggtggagctgatggtcgaaacgttcacagcctagggaattcctgaagaatgggaggcaggtgttgttgattatgagtgtgtaaaagaaaggggtagagagccgtcctcagatccgactactatgcaggtagccgctcgcccatgcccgcctggctgaatattgatgcatgcccatcaaggcaggcaggcatttctgtgcacgcaccaagcccacaatcttccacaacacacagcatgtaccaacgcacgcgtaaaagttggggtgctgccagtgcgtcatgccaggcatgatgtgctcctgcacatccgccatgatctcctccatcgtctcgggtgtttccggcgcctggtccgggagccgttccgccagatacccagacgccacctccgacctcacggggtacttttcgagcgtctgccggtagtcgacgatcgcgtccaccatggagtagccgaggcgccggaactggcgtgacggagggaggagagggaggagagagaggggggggggggggggggatgattacacgccagtctcacaacgcatgcaagacccgtttgattatgagtacaatcatgcactactagatggatgagcgccaggcataaggcacaccgacgttgatggcatgagcaactcccgcatcatatttcctattgtcctcacgccaagccggtcaccatccgcatgctcatattacagcgcacgcaccgcttcgtgatccaccgggtgaacgtagtcctcgacggaaacatctggctcgggcctcgtgctggcactccctcccatgccgacaacctttctgctgtcaccacgacccacgatgcaacgcgacacgacccggtgggactgatcggttcactgcacctgcatgcaattgtcacaagcgcatactccaatcgtatccgtttgatttctgtgaaaactcgctcgaccgcccgcgtcccgcaggcagcgatgacgtgtgcgtgacctgggtgtttcgtcgaaaggccagcaaccccaaatcgcaggcgatccggagattgggatctgatccgagcttggaccagatcccccacgatgcggcacgggaactgcatcgactcggcgcggaacccagctttcgtaaatgccagattggtgtccgataccttgatttgccatcagcgaaacaagacttcagcagcgagcgtatttggcgggcgtgctaccagggttgcatacattgcccatttctgtctggaccgctttaccggcgcagagggtgagttgatggggttggcaggcatcgaaacgcgcgtgcatggtgtgtgtgtctgttttcggctgcacaatttcaatagtcggatgggcgacggtagaattgggtgttgcgctcgcgtgcatgcctcgccccgtcgggtgtcatgaccgggactggaatcccccctcgcgaccctcctgctaacgctcccgactctcccgcccgcgcgcaggatagactctagttcaaccaatcgacacatatggcttccgcggcattcaccatgtcggcgtgccccgcgatgactggcagggcccctggggcacgtcgctccggacggccagtcgccacccgcctgaggtacgtattccagtgcctggtggccagctgcatcgacccctgcgaccagtaccgcagcagcgccagcctgagcttcctgggcgacaacggcttcgccagcctgttcggcagcaagcccttcatgagcaaccgcggccaccgccgcctgcgccgcgccagccacagcggcgaggccatggccgtggccctgcagcccgcccaggaggccggcaccaagaagaagcccgtgatcaagcagcgccgcgtggtggtgaccggcatgggcgtggtgacccccctgggccacgagcccgacgtgttctacaacaacctgctggacggcgtgagcggcatcagcgagatcgagaccttcgactgcacccagttccccacccgcatcgccggcgagatcaagagcttcagcaccgacggctgggtggcccccaagctgagcaagcgcatggacaagttcatgctgtacctgctgaccgccggcaagaaggccctggccgacggcggcatcaccgacgaggtgatgaaggagctggacaagcgcaagtgcggcgtgctgatcggcagcggcatgggcggcatgaaggtgttcaacgacgccatcgaggccctgcgcgtgagctacaagaagatgaaccccttctgcgtgcccttcgccaccaccaacatgggcagcgccatgctggccatggacctgggctggatgggccccaactacagcatcagcaccgcctgcgccaccagcaacttctgcatcctgaacgccgccaaccacatcatccgcggcgaggccgacatgatgctgtgcggcggcagcgacgccgtgatcatccccatcggcctgggcggcttcgtggcctgccgcgccctgagccagcgcaacagcgaccccaccaaggccagccgcccctgggacagcaaccgcgacggcttcgtgatgggcgagggcgccggcgtgctgctgctggaggagctggagcacgccaagaagcgcggcgccaccatctacgccgagttcctgggcggcagcttcacctgcgacgcctaccacatgaccgagccccaccccgagggcgccggcgtgatcctgtgcatcgagaaggccctggcccaggccggcgtgagcaaggaggacgtgaactacatcaacgcccacgccaccagcaccagcgccggcgacatcaaggagtaccaggccctggcccgctgcttcggccagaacagcgagctgcgcgtgaacagcaccaagagcatgatcggccacctgctgggcgccgccggcggcgtggaggccgtgaccgtggtgcaggccatccgcaccggctggattcaccccaacctgaacctggaggaccccgacaaggccgtggacgccaagctgctggtgggccccaagaaggagcgcctgaacgtgaaggtgggcctgagcaacagcttcggcttcggcggccacaacagcagcatcctgttcgccccctgcaacgtgtgactcgaggcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggaaagcttctgaagaatgggaggcaggtgttgttgattatgagtgtgtaaaagaaaggggtagagagccgtcctcagatccgactactatgcaggtagccgctcgcccatgcccgcctggctgaatattgatgcatgcccatcaaggcaggcaggcatttctgtgcacgcaccaagcccacaatcttccacaacacacagcatgtaccaacgcacgcgtaaaagttggggtgctgccagtgcgtcatgccaggcatgatgtgctcctgcacatccgccatgatctcctccatcgtctcgggtgtttccggcgcctggtccgggagccgttccgccagatacccagacgccacctccgacctcacggggtacttttcgagcgtctgccggtagtcgacgatcgcgtccaccatggagtagccgaggcgccggaactggcgtgacggagggaggagagggaggagagagaggggggggggggggggggatgattacacgccagtctcacaacgcatgcaagacccgtttgattatgagtacaatcatgcactactagatggatgagcgccaggcataaggcacaccgacgttgatggcatgagcaactcccgcatcatatttcctattgtcctcacgccaagccggtcaccatccgcatgctcatattacagcgcacgcaccgcttcgtgatccaccgggtgaacgtagtcctcgacggaaacatctggctcgggcctcgtgctggcactccctcccatgccgacaacctttctgctgtcaccacgacccacgatgcaacgcgacacgacccggtgggactgatcggttcactgcacctgcatgcaattgtcacaagcgcatactccaatcgtatccgtttgatttctgtgaaaactcgctcgaccgcccgcgtcccgcaggcagcgatgacgtgtgcgtgacctgggtgtttcgtcgaaaggccagcaaccccaaatcgcaggcgatccggagattgggatctgatccgagcttggaccagatcccccacgatgcggcacgggaactgcatcgactcggcgcggaacccagctttcgtaaatgccagattggtgtccgataccttgatttgccatcagcgaaacaagacttcagcagcgagcgtatttggcgggcgtgctaccagggttgcatacattgcccatttctgtctggaccgctttaccggcgcagagggtgagttgatggggttggcaggcatcgaaacgcgcgtgcatggtgtgtgtgtctgttttcggctgcacaatttcaatagtcggatgggcgacggtagaattgggtgttgcgctcgcgtgcatgcctcgccccgtcgggtgtcatgaccgggactggaatcccccctcgcgaccctcctgctaacgctcccgactctcccgcccgcgcgcaggatagactctagttcaaccaatcgacaactagtatggccaccgcctccaccttctccgccttcaacgcccgctgcggcgacctgcgccgctccgccggctccggcccccgccgccccgcccgccccctgcccgtgcgcgccgccatcaacgcctccgcccaccccaaggccaacggctccgccgtgaacctgaagtccggctccctgaacacccaggaggacacctcctcctccccccccccccgcgccttcctgaaccagctgcccgactggtccatgctgctgaccgccatcaccaccgtgttcgtggccgccgagaagcagtggaccatgcgcgaccgcaagtccaagcgccccgacatgctggtggactccgtgggcctgaagtccgtggtgctggacggcctggtgtcccgccagatcttctccatccgctcctacgagatcggcgccgaccgcaccgcctccatcgagaccctgatgaaccacctgcaggagacctccatcaaccactgcaagtccctgggcctgctgaacgacggcttcggccgcacccccggcatgtgcaagaacgacctgatctgggtgctgaccaagatgcagatcatggtgaaccgctaccccacctggggcgacaccgtggagatcaacacctggttctcccactccggcaagatcggcatggcctccgactggctgatcaccgactgcaacaccggcgagatcctgatccgcgccacctccgtgtgggccatgatgaaccagaagacccgccgcttctcccgcctgccctacgaggtgcgccaggagctgaccccccactacgtggactccccccacgtgatcgaggacaacgaccgcaagctgcacaagttcgacgtgaagaccggcgactccatccgcaagggcctgaccccccgctggaacgacctggacgtgaaccagcacgtgtccaacgtgaagtacatcggctggatcctggagtccatgcccatcgaggtgctggagacccaggagctgtgctccctgaccgtggagtaccgccgcgagtgcggcatggactccgtgctggagtccgtgaccgccatggacccctccgaggacgagggccgctcccagtacaagcacctgctgcgcctggaggacggcaccgacatcgtgaagggccgcaccgagtggcgccccaagaacgccggcaccaacggcgccatctccaccgccaagccctccaacggcaactccgtgtccatggactacaaggaccacgacggcgactacaaggaccacgacatcgactacaaggacgacgacgacaagtgactcgaggcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggaaagctgtatagggataacagggtaatgagctcagcgtctgcgtgttgggagctggagtcgtgggcttgacgacggcgctgcagctgttgcaggatgtgcctggcgtgcgcgttcacgtcgtggctgagaaatatggcgacgaaacgttgacggctggggccggcgggctgtggatgccatacgcattgggtacgcggccattggatgggattgataggcttatggagggataatagagtttttgccggatccaacgcatgtggatgcggtatcccggtgggctgaaagtgtggaaggatagtgcattggctattcacatgcactgcccaccccttttggcaggaaatgtgccggcatcgttggtgcaccgatggggaaaatcgacgttcgaccactacatgaagatttatacgtctgaagatgcagcgactgcgggtgcgaaacggatgacggtttggtcgtgtatgtcacagcatgtgctggatcttgcgggctaactccccctgccacggcccattgcaggtgtcatgttgactggagggtacgacctttcgtccgtcaaattcccagaggaggacccgctctgggccgacattgtgcccact

SEQ ID NO: 16 - DNA, 9010 bp, Artificial Sequence
  source: /note=“Description of Artificial Sequence Synthetic polynucleotide”
gtctaggttgcgaggtgactggccaggaagcagcaggcttggggtttggtgttctgatttctggtaatttgaggtttcattataagattctgtacggtcttgtttcgaaaacatgcaacaactccacacacacacactcctctcaactgagtctgcaggtttgacatctccgagttcccgaccaagtttgcggcgcagatcaccggcttctccgtggaggactgcgtggacaagaagaacgcgcggcggtacgacgacgcgctgtcgtacgcgatggtggcctccaagaaggccctgcgccaggcaggcctggagaaggacaagtgccccgagggctacggggcgctggacaagacgcgcacgggcgtgctggtcggctcgggcatgggcgggctgacggtcttccaggacggcgtcaaggcgctggtggagaagggctacaagaagatgagccccttcttcatcccctacgccatcaccaacatgggctccgcgctggtgggcatcgaccagggcttcatgggccccaactactccgtctccacagcctgcgcgacgtccaactacgcatttgtgaacgcggccaaccacatccgcaagggcgacgcggacgtcatggtcgtcggcggcaccgaggcctccatcgtgcccgtgggcctgggcggctttgtggcctgccgcgcgctgtccacgcgcaacgacgagcccaagcgcgcgagccggccgtgggacgagggccgcgacggctttggtaccccgctcccgtctggtcctcacgttcgtgtacggcctggatcccggaaagggcggatgcacgtggtgttgccccgccattggcgcccacgtttcaaagtccccggccagaaatgcacaggaccggcccggctcgcacaggccatgacgaatgcccagatttcgacagcaaaacaatctggaataatcgcaaccattcgcgttttgaacgaaacgaaaagacgctgtttagcacgtttccgatatcgtgggggccgaagcatgattggggggaggaaagcgtggccccaaggtagcccattctgtgccacacgccgacgaggaccaatccccggcatcagccttcatcgacggctgcgccgcacatataaagccggacgccttcccgacacgttcaaacagttttatttcctccacttcctgaatcaaacaaatcttcaaggaagatcctgctcttgagcaactagtatgttcgcgttctacttcctgacggcctgcatctccctgaagggcgtgttcggcgtctccccctcctacaacggcctgggcctgacgccccagatgggctgggacaactggaacacgttcgcctgcgacgtctccgagcagctgctgctggacacggccgaccgcatctccgacctgggcctgaaggacatgggctacaagtacatcatcctggacgactgctggtcctccggccgcgactccgacggcttcctggtcgccgacgagcagaagttccccaacggcatgggccacgtcgccgaccacctgcacaacaactccttcctgttcggcatgtactcctccgcgggcgagtacacgtgcgccggctaccccggctccctgggccgcgaggaggaggacgcccagttcttcgcgaacaaccgcgtggactacctgaagtacgacaactgctacaacaagggccagttcggcacgcccgagatctcctaccaccgctacaaggccatgtccgacgccctgaacaagacgggccgccccatcttctactccctgtgcaactggggccaggacctgaccttctactggggctccggcatcgcgaactcctggcgcatgtccggcgacgtcacggcggagttcacgcgccccgactcccgctgcccctgcgacggcgacgagtacgactgcaagtacgccggcttccactgctccatcatgaacatcctgaacaaggccgcccccatgggccagaacgcgggcgtcggcggctggaacgacctggacaacctggaggtcggcgtcggcaacctgacggacgacgaggagaaggcgcacttctccatgtgggccatggtgaagtcccccctgatcatcggcgcgaacgtgaacaacctgaaggcctcctcctactccatctactcccaggcgtccgtcatcgccatcaaccaggactccaacggcatccccgccacgcgcgtctggcgctactacgtgtccgacacggacgagtacggccagggcgagatccagatgtggtccggccccctggacaacggcgaccaggtcgtggcgctgctgaacggcggctccgtgtcccgccccatgaacacgaccctggaggagatcttcttcgactccaacctgggctccaagaagctgacctccacctgggacatctacgacctgtgggcgaaccgcgtcgacaactccacggcgtccgccatcctgggccgcaacaagaccgccaccggcatcctgtacaacgccaccgagcagtcctacaaggacggcctgtccaagaacgacacccgcctgttcggccagaagatcggctccctgtcccccaacgcgatcctgaacacgaccgtccccgcccacggcatcgcgttctaccgcctgcgcccctcctcctgatacaacttattacgtattctgaccggcgctgatgtggcgcggacgccgtcgtactctttcagactttactcttgaggaattgaacctttctcgcttgctggcatgtaaacattggcgcaattaattgtgtgatgaagaaagggtggcacaagatggatcgcgaatgtacgagatcgacaacgatggtgattgttatgaggggccaaacctggctcaatcttgtcgcatgtccggcgcaatgtgatccagcggcgtgactctcgcaacctggtagtgtgtgcgcaccgggtcgctttgattaaaactgatcgcattgccatcccgtcaactcacaagcctactctagctcccattgcgcactcgggcgcccggctcgatcaatgttctgagcggagggcgaagcgtcaggaaatcgtctcggcagctggaagcgcatggaatgcggagcggagatcgaatcaggatccgcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggaaagctgtagaattcgtgaaaactctctcgaccgcccgcgtcccgcaggcagcgatgacgtgtgcgtgacctgggtgtttcgtcgaaaggccagcaaccccaaatcgcaggcgatccggagattgggatctgatccgagcttggaccagatcccccacgatgcggcacgggaactgcatcgactcggcgcggaacccagctttcgtaaatgccagattggtgtccgataccttgatttgccatcagcgaaacaagacttcagcagcgagcgtatttggcgggcgtgctaccagggttgcatacattgcccatttctgtctggaccgctttaccggcgcagagggtgagttgatggggttggcaggcatcgaaacgcgcgtgcatggtgtgtgtgtctgttttcggctgcacaatttcaatagtcggatgggcgacggtagaattgggtgttgcgctcgcgtgcatgcctcgccccgtcgggtgtcatgaccgggactggaatcccccctcgcgaccctcctgctaacgctcccgactctcccgcccgcgcgcaggatagactctagttcaaccaatcgacaactagtaacaatggcttccgcggcattcaccatgtcggcgtgccccgcgatgactggcagggcccctggggcacgtcgctccggacggccagtcgccacccgcctgaggggctccaccttccagtgcctggtgaactcccacatcgacccctgcaaccagaacgtgtcctccgcctccctgtccttcctgggcgacaacggcttcggctccaaccccttccgctccaaccgcggccaccgccgcctgggccgcgcctcccactccggcgaggccatggccgtggccctgcagcccgcccaggaggtggccaccaagaagaagcccgccatcaagcagcgccgcgtggtggtgaccggcatgggcgtggtgacccccctgggccacgagcccgacgtgttctacaacaacctgctggacggcgtgtccggcatctccgagatcgagaccttcgactgcacccagttccccacccgcatcgccggcgagatcaagtccttctccaccgacggctgggtggcccccaagctgtccaagcgcatggacaagttcatgctgtacctgctgaccgccggcaagaaggccctggccgacgccggcatcaccgaggacgtgatgaaggagctggacaagcgcaagtgcggcgtgctgatcggctccggcatgggcggcatgaagctgttcaacgactccatcgaggccctgcgcgtgtcctacaagaagatgaaccccttctgcgtgcccttcgccaccaccaacatgggctccgccatgctggccatggacctgggctggatgggccccaactactccatctccaccgcctgcgccacctccaacttctgcatcctgaacgccgccaaccacatcatccgcggcgaggccgacatgatgctgtgcggcggctccgacgccgtgatcatccccatcggcctgggcggcttcgtggcctgccgcgccctgtcccagcgcaactccgaccccaccaaggcctcccgcccctgggactccaaccgcgacggcttcgtgatgggcgagggcgccggcgtgctgctgctggaggagctggagcacgccaagaagcgcggcgccaccatctacgccgagttcctgggcggctccttcacctgcgacgcctaccacatgaccgagccccaccccgacggcgccggcgtgatcctgtgcatcgagaaggccctggcccagtccggcgtgtcccgcgaggacgtgaactacatcaacgcccacgccacctccacccccgccggcgacatcaaggagtaccaggccctggcccactgcttcggccagaactccgagctgcgcgtgaactccaccaagtccatgatcggccacctgctgggcgccgccggcggcgtggaggccgtgaccgtgatccaggccatccgcaccggctggatccaccccaacctgaacctggaggaccccgacgaggccgtggacgccaagttcctggtgggccccaagaaggagcgcctgaacgtgaaggtgggcctgtccaactccttcggcttcggcggccacaactcctccatcctgttcgccccctacaacaccatgtacccctacgacgtgcccgactacgcctgatatcgaggcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggaaagcttgagacggtgaaaactcgctcgaccgcccgcgtcccgcaggcagcgatgacgtgtgcgtgacctgggtgtttcgtcgaaaggccagcaaccccaaatcgcaggcgatccggagattgggatctgatccgagcttggaccagatcccccacgatgcggcacgggaactgcatcgactcggcgcggaacccagctttcgtaaatgccagattggtgtccgataccttgatttgccatcagcgaaacaagacttcagcagcgagcgtatttggcgggcgtgctaccagggttgcatacattgcccatttctgtctggaccgctttaccggcgcagagggtgagttgatggggttggcaggcatcgaaacgcgcgtgcatggtgtgtgtgtctgttttcggctgcacaatttcaatagtcggatgggcgacggtagaattgggtgttgcgctcgcgtgcatgcctcgccccgtcgggtgtcatgaccgggactggaatcccccctcgcgaccctcctgctaacgctcccgactctcccgcccgcgcgcaggatagactctagttcaaccaatcgacaactagtaacaatggccaccgcctccaccttctccgccttcaacgcccgctgcggcgacctgcgccgctccgccggctccggcccccgccgccccgcccgccccctgcccgtgcgcgccgccatcggcaacgagcgcaactcctgcaaggtgatcaacggcaccaaggtgaaggacaccgagggcctgaagggctgctccaccctgcagggccagtccatgctggacgaccacttcggcctgcacggcctggtgttccgccgcaccttcgccatccgctgctacgaggtgggccccgaccgctccacctccatcatggccgtgatgaaccacctgcaggaggccgcccgcaaccacgccgagtccctgggcctgctgggcgacggcttcggcgagaccctggagatgtccaagcgcgacctgatctgggtggtgcgccgcacccacgtggccgtggagcgctaccccgcctggggcgacaccgtggaggtggaggcctgggtgggcgcctccggcaacaccggcatgcgccgcgacttcctggtgcgcgactgcaagaccggccacatcctgacccgctgcacctccgtgtccgtgatgatgaacatgcgcacccgccgcctgtccaagatcccccaggaggtgcgcgccgagatcgaccccctgttcatcgagaaggtggccgtgaaggagggcgagatcaagaagctgcagaagctgaacgactccaccgccgactacatccagggcggctggaccccccgctggaacgacctggacgtgaaccagcacgtgaacaacatcatctacgtgggctggatcttcaagtccgtgcccgactccatctccgagaaccaccacctgtcctccatcaccctggagtaccgccgcgagtgcacccgcggcaacaagctgcagtccctgaccaccgtgtgcggcggctcctccgaggccggcatcatctgcgagcacctgctgcagctggaggacggctccgaggtgctgcgcgcccgcaccgagtggcgccccaagcacaccgactccttccagggcatctccgagcgcttcccccagcaggagccccacaaggactacaaggaccacgacggcgactacaaggaccacgacatcgactacaaggacgacgacgacaagtgactcgaggcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggaaagcttgagctcgtgatgggcgagggcgcggccgtgctggtcatggagtcgctggagcacgcgcagaagcgtggcgcgaccatcctgggcgagtacctgggcggcgccatgacctgcgacgcgcaccacatgacggacccgcaccccgagggcctgggcgtgagcacctgcatccgcctggcgctcgaggacgccggcgtctcgcccgacgaggtcaactacgtcaacgcgcacgccacctccaccctggtgggcgacaaggccgaggtgcgcgcggtcaagtcggtctttggcgacatgaagggtatcaagatgaacgccaccaagagtatgatcgggcactgcctgggcgccgccggcggcatggaggccgtcgcgacgctcatggccatccgcaccggctgggtgcaccccaccatcaaccacgacaaccccatcgccgaggtcgatggcctggacgtcgtcgccaacgccaaggcccagcacgacatcaacgtcgccatctccaactccttcggctttggcgggcacaactccgtcgtcgcctttgcgcccttccgcgagtaggtgaagcgagcgtgctttgctgaggagggaggcggggtgcgagcgctctggccgtgcgcgcgatactctccccgcatgagcagactcctcgtgccacgcccgaatctacttgtcaacgagcaactgtgtgttttgtccgtggccaatcttattatttctccgactgtggccgtactctgtttggctgtgcaagcacc

SEQ ID NO: 17 - DNA, 6451 bp, Artificial Sequence
  source: /note=“Description of Artificial Sequence Synthetic polynucleotide”
ccctcaactgcgacgctgggaaccttctccgggcaggcgatgtgcgtgggtttgcctccttggcacggctctacaccgtcgagtacgccatgaggcggtgatggctgtgtcggttgccacttcgtccagagacggcaagtcgtccatcctctgcgtgtgtggcgcgacgctgcagcagtccctctgcagcagatgagcgtgactttggccatttcacgcactcgagtgtacacaatccatttttcttaaagcaaatgactgctgattgaccagatactgtaacgctgatttcgctccagatcgcacagatagcgaccatgttgctgcgtctgaaaatctggattccgaattcgaccctggcgctccatccatgcaacagatggcgacacttgttacaattcctgtcacccatcggcatggagcaggtccacttagattcccgatcacccacgcacatctcgctaatagtcattcgttcgtgtcttcgatcaatctcaagtgagtgtgcatggatcttggttgacgatgcggtatgggtttgcgccgctggctgcagggtctgcccaaggcaagctaacccagctcctctccccgacaatactctcgcaggcaaagccggtcacttgccttccagattgccaataaactcaattatggcctctgtcatgccatccatgggtctgatgaatggtcacgctcgtgtcctgaccgttccccagcctctggcgtcccctgccccgcccaccagcccacgccgcgcggcagtcgctgccaaggctgtctcggaggtaccctttcttgcgctatgacacttccagcaaaaggtagggcgggctgcgagacggcttcccggcgctgcatgcaacaccgatgatgcttcgaccccccgaagctccttcggggctgcatgggcgctccgatgccgctccagggcgagcgctgtttaaatagccaggcccccgattgcaaagacattatagcgagctaccaaagccatattcaaacacctagatcactaccacttctacacaggccactcgagcttgtgatcgcactccgctaagggggcgcctcttcctcttcgtttcagtcacaacccgcaaactctagaatatcaatgctgctgcaggccttcctgttcctgctggccggcttcgccgccaagatcagcgcctccatgacgaacgagacgtccgaccgccccctggtgcacttcacccccaacaagggctggatgaacgaccccaacggcctgtggtacgacgagaaggacgccaagtggcacctgtacttccagtacaacccgaacgacaccgtctgggggacgcccttgttctggggccacgccacgtccgacgacctgaccaactgggaggaccagcccatcgccatcgccccgaagcgcaacgactccggcgccttctccggctccatggtggtggactacaacaacacctccggcttcttcaacgacaccatcgacccgcgccagcgctgcgtggccatctggacctacaacaccccggagtccgaggagcagtacatctcctacagcctggacggcggctacaccttcaccgagtaccagaagaaccccgtgctggccgccaactccacccagttccgcgacccgaaggtcttctggtacgagccctcccagaagtggatcatgaccgcggccaagtcccaggactacaagatcgagatctactcctccgacgacctgaagtcctggaagctggagtccgcgttcgccaacgagggcttcctcggctaccagtacgagtgccccggcctgatcgaggtccccaccgagcaggaccccagcaagtcctactgggtgatgttcatctccatcaaccccggcgccccggccggcggctccttcaaccagtacttcgtcggcagcttcaacggcacccacttcgaggccttcgacaaccagtcccgcgtggtggacttcggcaaggactactacgccctgcagaccttcttcaacaccgacccgacctacgggagcgccctgggcatcgcgtgggcctccaactgggagtactccgccttcgtgcccaccaacccctggcgctcctccatgtccctcgtgcgcaagttctccctcaacaccgagtaccaggccaacccggagacggagctgatcaacctgaaggccgagccgatcctgaacatcagcaacgccggcccctggagccggttcgccaccaacaccacgttgacgaaggccaacagctacaacgtcgacctgtccaacagcaccggcaccctggagttcgagctggtgtacgccgtcaacaccacccagacgatctccaagtccgtgttcgcggacctctccctctggttcaagggcctggaggaccccgaggagtacctccgcatgggcttcgaggtgtccgcgtcctccttcttcctggaccgcgggaacagcaaggtgaagttcgtgaaggagaacccctacttcaccaaccgcatgagcgtgaacaaccagcccttcaagagcgagaacgacctgtcctactacaaggtgtacggcttgctggaccagaacatcctggagctgtacttcaacgacggcgacgtcgtgtccaccaacacctacttcatgaccaccgggaacgccctgggctccgtgaacatgacgacgggggtggacaacctgttctacatcgacaagttccaggtgcgcgaggtcaagtgacaattgacgcccgcgcggcgcacctgacctgttctctcgagggcgcctgttctgccttgcgaaacaagcccctggagcatgcgtgcatgatcgtctctggcgccccgccgcgcggtttgtcgccctcgcgggcgccgcggccgcgggggcgcattgaaattgttgcaaaccccacctgacagattgagggcccaggcaggaaggcgttgagatggaggtacaggagtcaagtaactgaaagtttttatgataactaacaacaaagggtcgtttctggccagcgaatgacaagaacaagattccacatttccgtgtagaggcttgccatcgaatgtgagcgggcgggccgcggacccgacaaaacccttacgacgtggtaagaaaaacgtggcgggcactgtccctgtagcctgaagaccagcaggagacgatcggaagcatcacagcacaggatcccgcgtctcgaacagagcgcgcagaggaacgctgaaggtctcgcctctgtcgcacctcagcgcggcatacaccacaataaccacctgacgaatgcgcttggttcttcgtccattagcgaagcgtccggttcacacacgtgccacgttggcgaggtggcaggtgacaatgatcggtggagctgatggtcgaaacgttcacagcctagggatatcgcctgctcaagcgggcgctcaacatgcagagcgtcagcgagacgggctgtggcgatcgcgagacggacgaggccgcctctgccctgtttgaactgagcgtcagcgctggctaaggggagggagactcatccccaggctcgcgccagggctctgatcccgtctcgggcggtgatcggcgcgcatgactacgacccaacgacgtacgagactgatgtcggtcccgacgaggagcgccgcgaggcactcccgggccaccgaccatgtttacaccgaccgaaagcactcgctcgtatccattccgtgcgcccgcacatgcatcatcttttggtaccgacttcggtcttgttttacccctacgacctgccttccaaggtgtgagcaactcgcccggacatgaccgagggtgatcatccggatccccaggccccagcagcccctgccagaatggctcgcgctttccagcctgcaggcccgtctcccaggtcgacgcaacctacatgaccaccccaatctgtcccagaccccaaacaccctccttccctgcttctctgtgatcgctgatcagcaacaactagtaacaatggccaccgcatccactttctcggcgttcaatgcccgctgcggcgacctgcgtcgctcggcgggctccgggccccggcgcccagcgaggcccctccccgtgcgcgggcgcgcctccagcctgagcccctccttcaagcccaagtccatccccaacggcggcttccaggtgaaggccaacgacagcgcccaccccaaggccaacggctccgccgtgagcctgaagagcggcagcctgaacacccaggaggacacctcctccagcccccccccccgcaccttcctgcaccagctgcccgactggagccgcctgctgaccgccatcaccaccgtgttcgtgaagtccaagcgccccgacatgcacgaccgcaagtccaagcgccccgacatgctggtggacagcttcggcctggagtccaccgtgcaggacggcctggtgttccgccagtccttctccatccgctcctacgagatcggcaccgaccgcaccgccagcatcgagaccctgatgaaccacctgcaggagacctccctgaaccactgcaagagcaccggcatcctgctggacggcttcggccgcaccctggagatgtgcaagcgcgacctgatctgggtggtgatcaagatgcagatcaaggtgaaccgctaccccgcctggggcgacaccgtggagatcaacacccgcttcagccgcctgggcaagatcggcatgggccgcgactggctgatctccgactgcaacaccggcgagatcctggtgcgcgccaccagcgcctacgccatgatgaaccagaagacccgccgcctgtccaagctgccctacgaggtgcaccaggagatcgtgcccctgttcgtggacagccccgtgatcgaggactccgacctgaaggtgcacaagttcaaggtgaagaccggcgacagcatccagaagggcctgacccccggctggaacgacctggacgtgaaccagcacgtgtccaacgtgaagtacatcggctggatcctggagagcatgcccaccgaggtgctggagacccaggagctgtgctccctggccctggagtaccgccgcgagtgcggccgcgactccgtgctggagagcgtgaccgccatggaccccagcaaggtgggcgtgcgctcccagtaccagcacctgctgcgcctggaggacggcaccgccatcgtgaacggcgccaccgagtggcgccccaagaacgccggcgccaacggcgccatctccaccggcaagaccagcaacggcaactccgtgtccatggactacaaggaccacgacggcgactacaaggaccacgacatcgactacaaggacgacgacgacaagtgactcgaggcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatttacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggaaagctgtatagggataacagggtaatgagctccagcgccatgccacgccctttgatggcttcaagtacgattacggtgttggattgtgtgtttgttgcgtagtgtgcatggtttagaataatacacttgatttcttgctcacggcaatctcggcttgtccgcaggttcaaccccatttcggagtctcaggtcagccgcgcaatgaccagccgctacttcaaggacttgcacgacaacgccgaggtgagctatgtttaggacttgattggaaattgtcgtcgacgcatattcgcgctccgcgacagcacccaagcaaaatgtcaagtgcgttccgatttgcgtccgcaggtcgatgttgtgatcgtcggcgccggatccgccggtctgtcctgcgcttacgagctgaccaagcaccctgacgtccgggtacgcgagctgagattcgattagacataaattgaagattaaacccgtagaaaaatttgatggtcgcgaaactgtgctcgattgcaagaaattgatcgtcctccactccgcaggtcgccatcatcgagcagggcgttgctcccggcggcggcgcctggctggggggacagctgttctcggccatgtgtgtacgtagaaggatgaatttcagctggttttcgttgcacagctgtttgtgcatgatttgtttcagactattgttgaatgtttttagatttcttaggatgcatgatttgtctgcatgcgact

SEQ ID NO: 18 - DNA, 9004 bp, Artificial Sequence
  source: /note=“Description of Artificial Sequence Synthetic polynucleotide”
gtctaggttgggaggcggctggcgaggaagcagcaggcttggggtttggtgttccgatttctggcaatttgaggtttcattgtgagattctatgcggtcttgtttcgaaaacatgcaacaactccacacacacacactcctctccaccaactctgcaggtttgacatctccgagttcccgaccaagtttgcggcgcagatcaccggcttctccgtggaggactgcgtggacaagaagaacgcgcggcggtacgacgacgcgctgtcgtacgcgatggtggcctccaagaaggccctgcgccaggcgggactggagaaggacaagtgccccgagggctacggagcgctggataagacgcgcgcgggcgtgctggtcggctcgggcatgggcgggctgacggtcttccaggacggcgtcaaggcgctggtggagaagggctacaagaagatgagccccttcttcatcccctacgccatcaccaacatgggctccgcgctggtgggcatcgaccagggcttcatggggcccaactactccgtctccacggcctgcgcgacctccaactacgcctttgtgaacgcggccaaccacatccgcaagggcgacgcggacgtcatggtcgtgggcggcaccgaggcctccatcgtgcccgtgggcctgggcggctttgtggcctgccgcgcgctgtccacgcgcaacgacgagcccaagcgcgcgagccggccgtgggacgagggccgcgacggcttcggtaccccgctcccgtctggtcctcacgttcgtgtacggcctggatcccggaaagggcggatgcacgtggtgttgccccgccattggcgcccacgtttcaaagtccccggccagaaatgcacaggaccggcccggctcgcacaggccatgacgaatgcccagatttcgacagcaaaacaatctggaataatcgcaaccattcgcgttttgaacgaaacgaaaagacgctgtttagcacgtttccgatatcgtgggggccgaagcatgattggggggaggaaagcgtggccccaaggtagcccattctgtgccacacgccgacgaggaccaatccccggcatcagccttcatcgacggctgcgccgcacatataaagccggacgccttcccgacacgttcaaacagttttatttcctccacttcctgaatcaaacaaatcttcaaggaagatcctgctcttgagcaactagtatgttcgcgttctacttcctgacggcctgcatctccctgaagggcgtgttcggcgtctccccctcctacaacggcctgggcctgacgccccagatgggctgggacaactggaacacgttcgcctgcgacgtctccgagcagctgctgctggacacggccgaccgcatctccgacctgggcctgaaggacatgggctacaagtacatcatcctggacgactgctggtcctccggccgcgactccgacggcttcctggtcgccgacgagcagaagttccccaacggcatgggccacgtcgccgaccacctgcacaacaactccttcctgttcggcatgtactcctccgcgggcgagtacacgtgcgccggctaccccggctccctgggccgcgaggaggaggacgcccagttcttcgcgaacaaccgcgtggactacctgaagtacgacaactgctacaacaagggccagttcggcacgcccgagatctcctaccaccgctacaaggccatgtccgacgccctgaacaagacgggccgccccatcttctactccctgtgcaactggggccaggacctgaccttctactggggctccggcatcgcgaactcctggcgcatgtccggcgacgtcacggcggagttcacgcgccccgactcccgctgcccctgcgacggcgacgagtacgactgcaagtacgccggcttccactgctccatcatgaacatcctgaacaaggccgcccccatgggccagaacgcgggcgtcggcggctggaacgacctggacaacctggaggtcggcgtcggcaacctgacggacgacgaggagaaggcgcacttctccatgtgggccatggtgaagtcccccctgatcatcggcgcgaacgtgaacaacctgaaggcctcctcctactccatctactcccaggcgtccgtcatcgccatcaaccaggactccaacggcatccccgccacgcgcgtctggcgctactacgtgtccgacacggacgagtacggccagggcgagatccagatgtggtccggccccctggacaacggcgaccaggtcgtggcgctgctgaacggcggctccgtgtcccgccccatgaacacgaccctggaggagatcttcttcgactccaacctgggctccaagaagctgacctccacctgggacatctacgacctgtgggcgaaccgcgtcgacaactccacggcgtccgccatcctgggccgcaacaagaccgccaccggcatcctgtacaacgccaccgagcagtcctacaaggacggcctgtccaagaacgacacccgcctgttcggccagaagatcggctccctgtcccccaacgcgatcctgaacacgaccgtccccgcccacggcatcgcgttctaccgcctgcgcccctcctcctgatacaacttattacgtattctgaccggcgctgatgtggcgcggacgccgtcgtactctttcagactttactcttgaggaattgaacctttctcgcttgctggcatgtaaacattggcgcaattaattgtgtgatgaagaaagggtggcacaagatggatcgcgaatgtacgagatcgacaacgatggtgattgttatgaggggccaaacctggctcaatcttgtcgcatgtccggcgcaatgtgatccagcggcgtgactctcgcaacctggtagtgtgtgcgcaccgggtcgctttgattaaaactgatcgcattgccatcccgtcaactcacaagcctactctagctcccattgcgcactcgggcgcccggctcgatcaatgttctgagcggagggcgaagcgtcaggaaatcgtctcggcagctggaagcgcatggaatgcggagcggagatcgaatcaggatccgcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggaaagctgtagaattcgtgaaaactctctcgaccgcccgcgtcccgcaggcagcgatgacgtgtgcgtgacctgggtgtttcgtcgaaaggccagcaaccccaaatcgcaggcgatccggagattgggatctgatccgagcttggaccagatcccccacgatgcggcacgggaactgcatcgactcggcgcggaacccagctttcgtaaatgccagattggtgtccgataccttgatttgccatcagcgaaacaagacttcagcagcgagcgtatttggcgggcgtgctaccagggttgcatacattgcccatttctgtctggaccgctttaccggcgcagagggtgagttgatggggttggcaggcatcgaaacgcgcgtgcatggtgtgtgtgtctgttttcggctgcacaatttcaatagtcggatgggcgacggtagaattgggtgttgcgctcgcgtgcatgcctcgccccgtcgggtgtcatgaccgggactggaatcccccctcgcgaccctcctgctaacgctcccgactctcccgcccgcgcgcaggatagactctagttcaaccaatcgacaactagtaacaatggcttccgcggcattcaccatgtcggcgtgccccgcgatgactggcagggcccctggggcacgtcgctccggacggccagtcgccacccgcctgaggggctccaccttccagtgcctggtgaactcccacatcgacccctgcaaccagaacgtgtcctccgcctccctgtccttcctgggcgacaacggcttcggctccaaccccttccgctccaaccgcggccaccgccgcctgggccgcgcctcccactccggcgaggccatggccgtggccctgcagcccgcccaggaggtggccaccaagaagaagcccgccatcaagcagcgccgcgtggtggtgaccggcatgggcgtggtgacccccctgggccacgagcccgacgtgttctacaacaacctgctggacggcgtgtccggcatctccgagatcgagaccttcgactgcacccagttccccacccgcatcgccggcgagatcaagtccttctccaccgacggctgggtggcccccaagctgtccaagcgcatggacaagttcatgctgtacctgctgaccgccggcaagaaggccctggccgacgccggcatcaccgaggacgtgatgaaggagctggacaagcgcaagtgcggcgtgctgatcggctccggcatgggcggcatgaagctgttcaacgactccatcgaggccctgcgcgtgtcctacaagaagatgaaccccttctgcgtgcccttcgccaccaccaacatgggctccgccatgctggccatggacctgggctggatgggccccaactactccatctccaccgcctgcgccacctccaacttctgcatcctgaacgccgccaaccacatcatccgcggcgaggccgacatgatgctgtgcggcggctccgacgccgtgatcatccccatcggcctgggcggcttcgtggcctgccgcgccctgtcccagcgcaactccgaccccaccaaggcctcccgcccctgggactccaaccgcgacggcttcgtgatgggcgagggcgccggcgtgctgctgctggaggagctggagcacgccaagaagcgcggcgccaccatctacgccgagttcctgggcggctccttcacctgcgacgcctaccacatgaccgagccccaccccgacggcgccggcgtgatcctgtgcatcgagaaggccctggcccagtccggcgtgtcccgcgaggacgtgaactacatcaacgcccacgccacctccacccccgccggcgacatcaaggagtaccaggccctggcccactgcttcggccagaactccgagctgcgcgtgaactccaccaagtccatgatcggccacctgctgggcgccgccggcggcgtggaggccgtgaccgtgatccaggccatccgcaccggctggatccaccccaacctgaacctggaggaccccgacgaggccgtggacgccaagttcctggtgggccccaagaaggagcgcctgaacgtgaaggtgggcctgtccaactccttcggcttcggcggccacaactcctccatcctgttcgccccctacaacaccatgtacccctacgacgtgcccgactacgcctgatatcgaggcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggaaagcttatcgcctgctcaagcgggcgctcaacatgcagagcgtcagcgagacgggctgtggcgatcgcgagacggacgaggccgcctctgccctgtttgaactgagcgtcagcgctggctaaggggagggagactcatccccaggctcgcgccagggctctgatcccgtctcgggcggtgatcggcgcgcatgactacgacccaacgacgtacgagactgatgtcggtcccgacgaggagcgccgcgaggcactcccgggccaccgaccatgtttacaccgaccgaaagcactcgctcgtatccattccgtgcgcccgcacatgcatcatcttttggtaccgacttcggtcttgttttacccctacgacctgccttccaaggtgtgagcaactcgcccggacatgaccgagggtgatcatccggatccccaggccccagcagcccctgccagaatggctcgcgctttccagcctgcaggcccgtctcccaggtcgacgcaacctacatgaccaccccaatctgtcccagaccccaaacaccctccttccctgcttctctgtgatcgctgatcagcaacaactagtaacaatggccaccgcctccaccttctccgccttcaacgcccgctgcggcgacctgcgccgctccgccggctccggcccccgccgccccgcccgccccctgcccgtgcgcgccgccatcggcaacgagcgcaactcctgcaaggtgatcaacggcaccaaggtgaaggacaccgagggcctgaagggctgctccaccctgcagggccagtccatgctggacgaccacttcggcctgcacggcctggtgttccgccgcaccttcgccatccgctgctacgaggtgggccccgaccgctccacctccatcatggccgtgatgaaccacctgcaggaggccgcccgcaaccacgccgagtccctgggcctgctgggcgacggcttcggcgagaccctggagatgtccaagcgcgacctgatctgggtggtgcgccgcacccacgtggccgtggagcgctaccccgcctggggcgacaccgtggaggtggaggcctgggtgggcgcctccggcaacaccggcatgcgccgcgacttcctggtgcgcgactgcaagaccggccacatcctgacccgctgcacctccgtgtccgtgatgatgaacatgcgcacccgccgcctgtccaagatcccccaggaggtgcgcgccgagatcgaccccctgttcatcgagaaggtggccgtgaaggagggcgagatcaagaagctgcagaagctgaacgactccaccgccgactacatccagggcggctggaccccccgctggaacgacctggacgtgaaccagcacgtgaacaacatcatctacgtgggctggatcttcaagtccgtgcccgactccatctccgagaaccaccacctgtcctccatcaccctggagtaccgccgcgagtgcacccgcggcaacaagctgcagtccctgaccaccgtgtgcggcggctcctccgaggccggcatcatctgcgagcacctgctgcagctggaggacggctccgaggtgctgcgcgcccgcaccgagtggcgccccaagcacaccgactccttccagggcatctccgagcgcttcccccagcaggagccccacaaggactacaaggaccacgacggcgactacaaggaccacgacatcgactacaaggacgacgacgacaagtgactcgaggcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggaaagcttgagctcgtgatgggcgagggcgcggccgtgctggtcatggagtcgctggagcacgcgcagaagcgcggcgcgaccatcctgggcgagtacctggggggcgccatgacctgcgacgcgcaccacatgacggacccgcaccccgagggcctgggcgtgagcacctgcatccgcctggcgctcgaggacgccggcgtctcgcccgacgaggtcaactacgtcaacgcgcacgccacctccaccctggtgggcgacaaggccgaggtgcgcgcggtcaagtcggtctttggcgacatgaagggcatcaagatgaacgccaccaagtccatgatcgggcactgcctgggcgccgccggcggcatggaggccgtcgccacgctcatggccatccgcaccggctgggtgcaccccaccatcaaccacgacaaccccatcgccgaggtcgacggcctggacgtcgtcgccaacgccaaggcccagcacaaaatcaacgtcgccatctccaactccttcggcttcggcgggcacaactccgtcgtcgcctttgcgcccttccgcgagtaggcggagcgagcgcgcttggctgaggagggaggcggggtgcgagccctttggctgcgcgcgatactctccccgcacgagcagactccacgcgcctgaatctacttgtcaacgagcaaccgtgtgttttgtccgtggccattcttattatttctccgactgtggccgtactctgtttggctgtgcaagcacc

SEQ ID NO: 19 - DNA, 5974 bp, Artificial Sequence
  source: /note=“Description of Artificial Sequence Synthetic polynucleotide”
cgcctggagctggtgcagagcatggggcagtttgcggaggagagggtgctccccgtgctgcaccccgtggacaagctgtggcagccgcaggacttcctgcccgaccccgagtcgcccgacttcgaggaccaggtggcggagctgcgcgcgcgcgccaaggacctgcccgacgagtactttgtggtgctggtgggcgacatgatcacggaggaggcgctgccgacctacatggccatgctcaacaccttggacggtgtgcgcgacgacacgggcgcggctgaccacccgtgggcgcgctggacgcggcagtgggtggccgaggagaaccggcacggcgacctgctgaacaagtactgttggctgacggggcgcgtcaacatgcgggccgtggaggtgaccatcaacaacctgatcaagagcggcatgaacccgcagacggacaacaacccttacttgggcttcgtctacacctccttccaggagcgcgccaccaagtaggtaccctttcttgcgctatgacacttccagcaaaaggtagggcgggctgcgagacggcttcccggcgctgcatgcaacaccgatgatgcttcgaccccccgaagctccttcggggctgcatgggcgctccgatgccgctccagggcgagcgctgtttaaatagccaggcccccgattgcaaagacattatagcgagctaccaaagccatattcaaacacctagatcactaccacttctacacaggccactcgagcttgtgatcgcactccgctaagggggcgcctcttcctcttcgtttcagtcacaacccgcaaactctagaatatcaatgctgctgcaggccttcctgttcctgctggccggcttcgccgccaagatcagcgcctccatgacgaacgagacgtccgaccgccccctggtgcacttcacccccaacaagggctggatgaacgaccccaacggcctgtggtacgacgagaaggacgccaagtggcacctgtacttccagtacaacccgaacgacaccgtctgggggacgcccttgttctggggccacgccacgtccgacgacctgaccaactgggaggaccagcccatcgccatcgccccgaagcgcaacgactccggcgccttctccggctccatggtggtggactacaacaacacctccggcttcttcaacgacaccatcgacccgcgccagcgctgcgtggccatctggacctacaacaccccggagtccgaggagcagtacatctcctacagcctggacggcggctacaccttcaccgagtaccagaagaaccccgtgctggccgccaactccacccagttccgcgacccgaaggtcttctggtacgagccctcccagaagtggatcatgaccgcggccaagtcccaggactacaagatcgagatctactcctccgacgacctgaagtcctggaagctggagtccgcgttcgccaacgagggcttcctcggctaccagtacgagtgccccggcctgatcgaggtccccaccgagcaggaccccagcaagtcctactgggtgatgttcatctccatcaaccccggcgccccggccggcggctccttcaaccagtacttcgtcggcagcttcaacggcacccacttcgaggccttcgacaaccagtcccgcgtggtggacttcggcaaggactactacgccctgcagaccttcttcaacaccgacccgacctacgggagcgccctgggcatcgcgtgggcctccaactgggagtactccgccttcgtgcccaccaacccctggcgctcctccatgtccctcgtgcgcaagttctccctcaacaccgagtaccaggccaacccggagacggagctgatcaacctgaaggccgagccgatcctgaacatcagcaacgccggcccctggagccggttcgccaccaacaccacgttgacgaaggccaacagctacaacgtcgacctgtccaacagcaccggcaccctggagttcgagctggtgtacgccgtcaacaccacccagacgatctccaagtccgtgttcgcggacctctccctctggttcaagggcctggaggaccccgaggagtacctccgcatgggcttcgaggtgtccgcgtcctccttcttcctggaccgcgggaacagcaaggtgaagttcgtgaaggagaacccctacttcaccaaccgcatgagcgtgaacaaccagcccttcaagagcgagaacgacctgtcctactacaaggtgtacggcttgctggaccagaacatcctggagctgtacttcaacgacggcgacgtcgtgtccaccaacacctacttcatgaccaccgggaacgccctgggctccgtgaacatgacgacgggggtggacaacctgttctacatcgacaagttccaggtgcgcgaggtcaagtgacaattgacggagcgtcgtgcgggagggagtgtgccgagcggggagtcccggtctgtgcgaggcccggcagctgacgctggcgagccgtacgccccgagggtccccctcccctgcaccctcttccccttccctctgacggccgcgcctgttcttgcatgttcagcgacggatcccgcgtctcgaacagagcgcgcagaggaacgctgaaggtctcgcctctgtcgcacctcagcgcggcatacaccacaataaccacctgacgaatgcgcttggttcttcgtccattagcgaagcgtccggttcacacacgtgccacgttggcgaggtggcaggtgacaatgatcggtggagctgatggtcgaaacgttcacagcctagggatatcgaattcggccgacaggacgcgcgtcaaaggtgctggtcgtgtatgccctggccggcaggtcgttgctgctgctggttagtgattccgcaaccctgattttggcgtcttattttggcgtggcaaacgctggcgcccgcgagccgggccggcggcgatgcggtgccccacggctgccggaatccaagggaggcaagagcgcccgggtcagttgaagggctttacgcgcaaggtacagccgctcctgcaaggctgcgtggtggaattggacgtgcaggtcctgctgaagttcctccaccgcctcaccagcggacaaagcaccggtgtatcaggtccgtgtcatccactctaaagaactcgactacgacctactgatggccctagattcttcatcaaaaacgcctgagacacttgcccaggattgaaactccctgaagggaccaccaggggccctgagttgttccttccccccgtggcgagctgccagccaggctgtacctgtgatcgaggctggcgggaaaataggcttcgtgtgctcaggtcatgggaggtgcaggacagctcatgaaacgccaacaatcgcacaattcatgtcaagctaatcagctatttcctcttcacgagctgtaattgtcccaaaattctggtctaccgggggtgatccttcgtgtacgggcccttccctcaaccctaggtatgcgcgcatgcggtcgccgcgcaactcgcgcgagggccgagggtttgggacgggccgtcccgaaatgcagttgcacccggatgcgtggcaccttttttgcgataatttatgcaatggactgctctgcaaaattctggctctgtcgccaaccctaggatcagcggcgtaggatttcgtaatcattcgtcctgatggggagctaccgactaccctaatatcagcccgactgcctgacgccagcgtccacttttgtgcacacattccattcgtgcccaagacatttcattgtggtgcgaagcgtccccagttacgctcacctgtttcccgacctccttactgttctgtcgacagagcgggcccacaggccggtcgcagccactagtatggctatcaagacgaacaggcagcctgtggagaagcctccgttcacgatcgggacgctgcgcaaggccatccccgcgcactgtttcgagcgctcggcgcttcgtgggcgcgcccccaaggccaacggcagcgccgtgagcctgaagtccggcagcctgaacaccctggaggacccccccagcagcccccccccccgcaccttcctgaaccagctgcccgactggagccgcctgcgcaccgccatcaccaccgtgttcgtggccgccgagaagcagttcacccgcctggaccgcaagagcaagcgccccgacatgctggtggactggttcggcagcgagaccatcgtgcaggacggcctggtgttccgcgagcgcttcagcatccgcagctacgagatcggcgccgaccgcaccgccagcatcgagaccctgatgaaccacctgcaggacaccagcctgaaccactgcaagagcgtgggcctgctgaacgacggcttcggccgcacccccgagatgtgcacccgcgacctgatctgggtgctgaccaagatgcagatcgtggtgaaccgctaccccacctggggcgacaccgtggagatcaacagctggttcagccagagcggcaagatcggcatgggccgcgagtggctgatcagcgactgcaacaccggcgagatcctggtgcgcgccaccagcgcctgggccatgatgaaccagaagacccgccgcttcagcaagctgccctgcgaggtgcgccaggagatcgccccccacttcgtggacgccccccccgtgatcgaggacaacgaccgcaagctgcacaagttcgacgtgaagaccggcgacagcatctgcaagggcctgacccccggctggaacgacttcgacgtgaaccagcacgtgagcaacgtgaagtacatcggctggattctggagagcatgcccaccgaggtgctggagacccaggagctgtgcagcctgaccctggagtaccgccgcgagtgcggccgcgagagcgtggtggagagcgtgaccagcatgaaccccagcaaggtgggcgaccgcagccagtaccagcacctgctgcgcctggaggacggcgccgacatcatgaagggccgcaccgagtggcgccccaagaacgccggcaccaaccgcgccatcagcacctgattaattaactcgaggcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggaaagcttgagctccagccacggcaacaccgcgcgccttgcggccgagcacggcgacaagaacctgagcaagatctgcgggctgatcgccagcgacgagggccggcacgagatcgcctacacgcgcatcgtggacgagttcttccgcctcgaccccgagggcgccgtcgccgcctacgccaacatgatgcgcaagcagatcaccatgcccgcgcacctcatggacgacatgggccacggcgaggccaacccgggccgcaacctcttcgccgacttctccgcggtcgccgagaagatcgacgtctacgacgccgaggactactgccgcatcctggagcacctcaacgcgcgctggaaggtggacgagcgccaggtcagcggccaggccgccgcggaccaggagtacgtcctgggcctgccccagcgcttccggaaactcgccgagaagaccgccgccaagcgcaagcgcgtcgcgcgcaggcccgtcgccttctcctgga

SEQ ID NO: 20 - DNA, 8313 bp, Artificial Sequence
  source: /note=“Description of Artificial Sequence Synthetic polynucleotide”
ctcaccgcgtgaattgctgtcccaaacgtaagcatcatcgtggctcggtcacgcgatcctggatccggggatcctagaccgctggtggagagcgctgccgtcggattggtggcaagtaagattgcgcaggttggcgaagggagagaccaaaaccggaggctggaagcgggcacaacatcgtattattgcgtatagtagagcagtggcagtcgcatttcgaggtccgcaacggatctcgcaagctcgctacgctcacagtaggagaaaggggaccactgcccctgccagaatggtcgcgaccctctccctcgccggccccgcctgcaacacgcagtgcgtatccggcaagcgggctgtcgccttcaaccgcccccatgttggcgtccgggctcgatcaggtgcgctgaggggggtttggtgtgcccgcgcctctgggcccgtgtcggccgtgcggacgtggggccctgggcagtggatcagcagggtttgcgtgcaaatgcctataccggcgattgaatagcgatgaacgggatacggttgcgctcactccatgcccatgcgaccccgtttctgtccgccagccgtggtcgcccgggctgcgaagcgggaccccacccagcgcattgtgatcaccggaatgggcgtggggtaccctttcttgcgctatgacacttccagcaaaaggtagggcgggctgcgagacggcttcccggcgctgcatgcaacaccgatgatgcttcgaccccccgaagctccttcggggctgcatgggcgctccgatgccgctccagggcgagcgctgtttaaatagccaggcccccgattgcaaagacattatagcgagctaccaaagccatattcaaacacctagatcactaccacttctacacaggccactcgagcttgtgatcgcactccgctaagggggcgcctcttcctcttcgtttcagtcacaacccgcaaactctagaatatcaatgatcgagcaggacggcctccacgccggctcccccgccgcctgggtggagcgcctgttcggctacgactgggcccagcagaccatcggctgctccgacgccgccgtgttccgcctgtccgcccagggccgccccgtgctgttcgtgaagaccgacctgtccggcgccctgaacgagctgcaggacgaggccgcccgcctgtcctggctggccaccaccggcgtgccctgcgccgccgtgctggacgtggtgaccgaggccggccgcgactggctgctgctgggcgaggtgcccggccaggacctgctgtcctcccacctggcccccgccgagaaggtgtccatcatggccgacgccatgcgccgcctgcacaccctggaccccgccacctgccccttcgaccaccaggccaagcaccgcatcgagcgcgcccgcacccgcatggaggccggcctggtggaccaggacgacctggacgaggagcaccagggcctggcccccgccgagctgttcgcccgcctgaaggcccgcatgcccgacggcgaggacctggtggtgacccacggcgacgcctgcctgcccaacatcatggtggagaacggccgcttctccggcttcatcgactgcggccgcctgggcgtggccgaccgctaccaggacatcgccctggccacccgcgacatcgccgaggagctgggcggcgagtgggccgaccgcttcctggtgctgtacggcatcgccgcccccgactcccagcgcatcgccttctaccgcctgctggacgagttcttctgacaattggcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggaaagctgtatagggataaaagcttatagcgactgctaccccccgaccatgtgccgaggcagaaattatatacaagaagcagatcgcaattaggcacatcgctttgcattatccacacactattcatcgctgctgcggcaaggctgcagagtgtatttttgtggcccaggagctgagtccgaagtcgacgcgacgagcggcgcaggatccgacccctagacgagcactgtcattttccaagcacgcagctaaatgcgctgagaccgggtctaaatcatccgaaaagtgtcaaaatggccgattgggttcgcctaggacaatgcgctgcggattcgctcgagtccgctgccggccaaaaggcggtggtacaggaaggcgcacggggccaaccctgcgaagccgggggcccgaacgccgaccgccggccttcgatctcgggtgtccccctcgtcaatttcctctctcgggtgcagccacgaaagtcgtgacgcaggtcacgaaatccggttacgaaaaacgcaggtcttcgcaaaaacgtgagggtttcgcgtctcgccctagctattcgtatcgccgggtcagacccacgtgcagaaaagcccttgaataacccgggaccgtggttaccgcgccgcctgcaccagggggcttatataagcccacaccacacctgtctcaccacgcatttctccaactcgcgacttttcggaagaaattgttatccacctagtatagactgccacctgcaggaccttgtgtcttgcagtttgtattggtcccggccgtcgagcacgacagatctgggctagggttggcctggccgctcggcactcccctttagccgcgcgcatccgcgttccagaggtgcgattcggtgtgtggagcattgtcatgcgcttgtgggggtcgttccgtgcgcggcgggtccgccatgggcgccgacctgggccctagggtttgttttcgggccaagcgagcccctctcacctcgtcgcccccccgcattccctctctcttgcagccactagtatggctatcaagacgaacaggcagcctgtggagaagcctccgttcacgatcgggacgctgcgcaaggccatccccgcgcactgtttcgagcgctcggcgcttcgtgggcgcgcccagctgcccgactggagccgcctgctgaccgccatcaccaccgtgttcgtgaagtccaagcgccccgacatgcacgaccgcaagtccaagcgccccgacatgctggtggacagcttcggcctggagtccaccgtgcaggacggcctggtgttccgccagtccttctccatccgctcctacgagatcggcaccgaccgcaccgccagcatcgagaccctgatgaaccacctgcaggagacctccctgaaccactgcaagagcaccggcatcctgctggacggcttcggccgcaccctggagatgtgcaagcgcgacctgatctgggtggtgattaagatgcagatcaaggtgaaccgctaccccgcctggggcgacaccgtggagatcaacacccgcttcagccgcctgggcaagatcggcatgggccgcgactggctgatctccgactgcaacaccggcgagatcctggtgcgcgccaccagcgcctacgccatgatgaaccagaagacccgccgcctgtccaagctgccctacgaggtgcaccaggagatcgtgcccctgttcgtggacagccccgtgatcgaggactccgacctgaaggtgcacaagttcaaggtgaagaccggcgacagcatccagaagggcctgacccccggctggaacgacctggacgtgaaccagcacgtgtccaacgtgaagtacatcggctggatcctggagagcatgcccaccgaggtgctggagacccaggagctgtgctccctggccctggagtaccgccgcgagtgcggccgcgactccgtgctggagagcgtgaccgccatggaccccagcaaggtgggcgtgcgctcccagtaccagcacctgctgcgcctggaggacggcaccgccatcgtgaacggcgccaccgagtggcgccccaagaacgccggcgccaacggcgccatctccaccggcaagaccagcaacggcaactccgtgtccatggactacaaggaccacgacggcgactacaaggaccacgacatcgactacaaggacgacgacgacaagtgactcgagagcgtccagcgtgtgggatgaagggtgcgatggaacggggctgccgccccccctctgggcatctagctctgcaccgcacgccaggaagcccaagccaggccccgtcacactccctcgctgaagtgttccccccctgccccacactcatccaggtatcaacgccatcatgttctacgtccccgtcatcttcaactccctggggagcgggcgccgcgcgtcgctgctgaacaccatcatcatcaacgccgtcaactttgttaattaagaattcggccgacaggacgcgcgtcaaaggtgctggtcgtgtatgccctggccggcaggtcgttgctgctgctggttagtgattccgcaaccctgattttggcgtcttattttggcgtggcaaacgctggcgcccgcgagccgggccggcggcgatgcggtgccccacggctgccggaatccaagggaggcaagagcgcccgggtcagttgaagggctttacgcgcaaggtacagccgctcctgcaaggctgcgtggtggaattggacgtgcaggtcctgctgaagttcctccaccgcctcaccagcggacaaagcaccggtgtatcaggtccgtgtcatccactctaaagaactcgactacgacctactgatggccctagattcttcatcaaaaacgcctgagacacttgcccaggattgaaactccctgaagggaccaccaggggccctgagttgttccttccccccgtggcgagctgccagccaggctgtacctgtgatcgaggctggcgggaaaataggcttcgtgtgctcaggtcatgggaggtgcaggacagctcatgaaacgccaacaatcgcacaattcatgtcaagctaatcagctatttcctcttcacgagctgtaattgtcccaaaattctggtctaccgggggtgatccttcgtgtacgggcccttccctcaaccctaggtatgcgcgcatgcggtcgccgcgcaactcgcgcgagggccgagggtttgggacgggccgtcccgaaatgcagttgcacccggatgcgtggcaccttttttgcgataatttatgcaatggactgctctgcaaaattctggctctgtcgccaaccctaggatcagcggcgtaggatttcgtaatcattcgtcctgatggggagctaccgactaccctaatatcagcccgactgcctgacgccagcgtccacttttgtgcacacattccattcgtgcccaagacatttcattgtggtgcgaagcgtccccagttacgctcacctgtttcccgacctccttactgttctgtcgacagagcgggcccacaggccggtcgcagcccatatggcttccgcggcattcaccatgtcggcgtgccccgcgatgactggcagggcccctggggcacgtcgctccggacggccagtcgccacccgcctgaggtacgtattccagtgcctggtggccagctgcatcgacccctgcgaccagtaccgcagcagcgccagcctgagcttcctgggcgacaacggcttcgccagcctgttcggcagcaagcccttcatgagcaaccgcggccaccgccgcctgcgccgcgccagccacagcggcgaggccatggccgtggccctgcagcccgcccaggaggccggcaccaagaagaagcccgtgatcaagcagcgccgcgtggtggtgaccggcatgggcgtggtgacccccctgggccacgagcccgacgtgttctacaacaacctgctggacggcgtgagcggcatcagcgagatcgagaccttcgactgcacccagttccccacccgcatcgccggcgagatcaagagcttcagcaccgacggctgggtggcccccaagctgagcaagcgcatggacaagttcatgctgtacctgctgaccgccggcaagaaggccctggccgacggcggcatcaccgacgaggtgatgaaggagctggacaagcgcaagtgcggcgtgctgatcggcagcggcatgggcggcatgaaggtgttcaacgacgccatcgaggccctgcgcgtgagctacaagaagatgaaccccttctgcgtgcccttcgccaccaccaacatgggcagcgccatgctggccatggacctgggctggatgggccccaactacagcatcagcaccgcctgcgccaccagcaacttctgcatcctgaacgccgccaaccacatcatccgcggcgaggccgacatgatgctgtgcggcggcagcgacgccgtgatcatccccatcggcctgggcggcttcgtggcctgccgcgccctgagccagcgcaacagcgaccccaccaaggccagccgcccctgggacagcaaccgcgacggcttcgtgatgggcgagggcgccggcgtgctgctgctggaggagctggagcacgccaagaagcgcggcgccaccatctacgccgagttcctgggcggcagcttcacctgcgacgcctaccacatgaccgagccccaccccgagggcgccggcgtgatcctgtgcatcgagaaggccctggcccaggccggcgtgagcaaggaggacgtgaactacatcaacgcccacgccaccagcaccagcgccggcgacatcaaggagtaccaggccctggcccgctgcttcggccagaacagcgagctgcgcgtgaacagcaccaagagcatgatcggccacctgctgggcgccgccggcggcgtggaggccgtgaccgtggtgcaggccatccgcaccggctggattcaccccaacctgaacctggaggaccccgacaaggccgtggacgccaagctgctggtgggccccaagaaggagcgcctgaacgtgaaggtgggcctgagcaacagcttcggcttcggcggccacaacagcagcatcctgttcgccccctgcaacgtgtgactcgaggcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggaaagcttgagctccacctgcatccgcctggcgctcgaggacgccggcgtctcgcccgacgaggtcaactacgtcaacgcgcacgccacctccaccctggtgggcgacaaggccgaggtgcgcgcggtcaagtcggtctttggcgacatgaagggcatcaagatgaacgccaccaagtccatgatcgggcactgcctgggcgccgccggcggcatggaggccgtcgccacgctcatggccatccgcaccggctgggtgcaccccaccatcaaccacgacaaccccatcgccgaggtcgacggcctggacgtcgtcgccaacgccaaggcccagcacaaaatcaacgtcgccatctccaactccttcggcttcggcgggcacaactccgtcgtcgcctttgcgcccttccgcgagtaggcggagcgagcgcgcttggctgaggagggaggcggggtgcgagccctttggctgcgcgcgatactctccccgcacgagcagactccacgcgcctgaatctacttgtcaacgagcaaccgtgtgttttgtccgtggccattcttattatttctccgactgtggccgtactctgtttggctgtgcaagcacc

(19) United States
(12) Patent Application Publication
(10) Pub. No.: US 2018/0216144 A1
(43) Pub. Date: Aug. 2, 2018

(54) TRIGLYCERIDE OILS HAVING ASYMMETRIC TRIGLYCERIDE MOLECULES

(71) Applicant: Corbion Biotech, Inc., South San Francisco, CA (US)

(72) Inventor: Walter Rakitsky, San Diego, CA (US)

(73) Assignee: Corbion Biotech, Inc., South San Francisco, CA (US)

(21) Appl. No.: 15/763,784

(22) PCT Filed: Sep. 27, 2016
(86) PCT No.: PCT/US2016/053979

§ 371 Date: Mar. 27, 2018

Related U.S. Application Data
(60) Provisional application No. 62/237,102, filed on Oct. 5, 2015.
(60) Provisional application No. 62/233,907, filed on Sep. 28, 2015.

(51) Int. Cl. C12P 7/64; C11C 3/12; C11B 7/00

(52) U.S. Cl. CPC C12P 7/6463; C11C 3/123; G01N 2030/027; C11B 7/0058; C11B 7/0075

(57) ABSTRACT

Triglyceride oils having one or more populations of asymmetric triglyceride molecules are provided. Asymmetric triglyceride molecule populations are triglyceride molecules that consist of a C8:0 fatty acid or a C10:0 fatty acid at the sn-1 position and the sn-2 position, and C16:0 or C18:0 at the sn-3 position. Another population of asymmetric triglyceride molecules are triglyceride molecules that consist of a C16:0 fatty acid or a C18:0 fatty acid at the sn-1 position and the sn-2 position, and C8:0 or C10:0 fatty acid at the sn-3 position. Methods of producing triglyceride oils and using the same are provided using sucrose invertase and hydrogenation of the triglyceride oil. Triglyceride molecules are produced by using recombinant DNA techniques to produce oleaginous recombinant cells.

### CROSS-REFERENCE TO RELATED APPLICATIONS

This application claims the benefit under 35 U.S.C. 119(e) of US provisional patent application Nos. 62/233,907, filed Sep. 28, 2015; and 62/237,102, filed Oct. 5, 2015, the disclosures of which are incorporated herein by reference in their entirety for all purposes.

### REFERENCE TO A SEQUENCE LISTING

This application includes a sequence listing appended hereto.

### FIELD OF THE INVENTION

Embodiments of the present invention relate to oils/fats, fuels, foods, and oleochemicals and their production from cultures of genetically engineered cells. Specific embodiments relate to oils with a high content of triglycerides bearing fatty acyl groups upon the glycerol backbone in particular regiospecific patterns, and with particular structuring characteristics, and products produced from such oils.

### BACKGROUND OF THE INVENTION

In the early 1990's reduced calorie fats were produced using a combination of short or medium and long chain fatty acids on a glycerol backbone (Salatrim/Caprinen). Although the metabolic calorie content ranged from 4.5-5.5 calories per gram, which was a significant reduction from the nine calories per gram of typical oils and fats, the functional properties of these fats were inferior to typical structuring fats like specific palm fractions, interesterified fats and cocoa butter due to their inability to form structures or stable crystal forms in the presence of liquid oils essential to generate acceptable textural properties common to many food products such as chocolate confections, margarines/spreads, and bakery coatings and fillings.

PCT Publications WO2008/151149, WO2010/063032, WO2011/150410, WO2011/150411, WO2012/061647, and WO2012/106560 disclose oils and methods for producing those oils in microbes, including microalgae. These publications also describe the use of such oils to make oleochemicals and fuels.

Tempering is a process of converting a fat into a desired polymorphic form by manipulation of the temperature of the fat or fat-containing substance, commonly used in chocolate making.

Certain enzymes of the fatty acyl-CoA elongation pathway function to extend the length of fatty acyl-CoA molecules. Elongase-complex enzymes extend fatty acyl-CoA molecules in 2 carbon additions, for example myristoyl-CoA to palmitoyl-CoA, stearoyl-CoA to arachidyl-CoA, or oleoyl-CoA to eicosanoyl-CoA, eicosanoyl-CoA to erucyl-CoA. In addition, elongase enzymes also extend acyl chain length in 2 carbon increments. KCS enzymes condense acyl-CoA molecules with two carbons from malonyl-CoA to form beta-ketoacyl-CoA. KCS and elongases may show specificity for condensing acyl substrates of particular carbon length, modification (such as hydroxylation), or degree of saturation. For example, the jojoba (Simmondsia chinensis) beta-ketoacyl-CoA synthase has been demonstrated to prefer monounsaturated and saturated C18- and C20-CoA substrates to elevate production of erucic acid in transgenic plants (Lassner et al., Plant Cell, 1996, Vol 8(2), pp. 281-292), whereas specific elongase enzymes of Trypanosoma brucei show preference for elongating short and midchain saturated CoA substrates (Lee et al., Cell, 2006, Vol 126(4), pp. 691-9).

The type II fatty acid biosynthetic pathway employs a series of reactions catalyzed by soluble proteins with intermediates shuttled between enzymes as thioesters of acyl carrier protein (ACP). By contrast, the type I fatty acid biosynthetic pathway uses a single, large multifunctional polypeptide.

The oleaginous, non-photosynthetic alga, Prototheca moriformis, stores copious amounts of triacylglyceride oil under conditions when the nutritional carbon supply is in excess, but cell division is inhibited due to limitation of other essential nutrients. Bulk biosynthesis of fatty acids with carbon chain lengths up to C18 occurs in the plastids; fatty acids are then exported to the endoplasmic reticulum where (if it occurs) elongation past C18 and incorporation into triacylglycerides (TAGs) is believed to occur. Lipids are stored in large cytoplasmic organelles called lipid bodies until environmental conditions change to favor growth, whereupon they are mobilized to provide energy and carbon molecules for anabolic metabolism.

### SUMMARY OF THE INVENTION

In one aspect, the present invention provides a method of preparing a triglyceride oil, in which the triglyceride oil comprises a first population of asymmetric triglyceride molecules and/or a second population of asymmetric triglyceride molecules, the first population comprising triglyceride molecules consisting of a C8:0 fatty acid or a C10:0 fatty acid at the sn-1 position and the sn-2 position, and C14:0, C16:0 or C18:0 at the sn-3 position, the second population comprising triglyceride molecules consisting of a C14:0, C16:0 fatty acid or a C18:0 fatty acid at the sn-1 position and the sn-2 position, and C8:0 or C10:0 fatty acid at the sn-3 position, wherein the method comprises: (a) obtaining a triglyceride oil isolated from a recombinant microalgal cell, wherein the recombinant microalgal cell comprises an exogenous gene encoding an active sucrose invertase; and (b) hydrogenating the triglyceride oil to produce the asymmetric triglyceride molecules.

In some embodiments of the method, the first population or the second population of triglyceride molecules is enriched by fractionation or preparative liquid chromatography.

In some cases, the first population of triglyceride molecules comprises at least 20%, at least 30% or at least 40% of all triglyceride molecules. In some cases, the second population of triglyceride molecules comprises at least 15%, 20% or 25% of all triglyceride molecules. In some cases, the first and second populations of triglyceride molecules together comprises at least 40%, 45%, 50% or 60% of all triglyceride molecules.

In some embodiments, the triglyceride oil has less than 9 kilocalories per gram or 4 to 8 kilocalories per gram. In some cases, the triglyceride oil has 5 to 8 kilocalories per gram, and in some cases, the triglyceride oil has 6 to 8 kilocalories per gram. Without being being bound to the mechanism of the calorie reduction, the reduction in the kilocalories per gram arises from the shorter chain length of the fatty acid residues of the TAG or because triacylglycerides in which there is a short chain fatty acid(s) (C8:0 and C10:0) and a mid and long chain fatty acid (C14:0, C16:0 and C18:0) on the glycerol backbone have been shown to be less readily metabolize during digestion.

In various embodiments, the triglyceride oil is a solid at ambient temperature and pressure. In a preferred embodiment, the triglyceride oil is a structuring fat, laminating fat or a coating fat. In some cases, the melting curve of the asymmetric triglyceride oil has one or more melting point at about 17° C., 31° C., and 37° C. In some embodiments, the triglyceride oil forms a crystalline polymorph of the β or β′ form.

In various embodiments of the present invention, the recombinant microalgal cell further comprises one or more exogenous gene encoding a fatty acyl-ACP thioesterase, a ketoacyl-ACP synthase, or a desaturase enzyme. In some embodiments, the recombinant microalgal cell further comprises (or also comprises) one or more exogenous gene that disrupts the expression of an endogenous gene encoding a fatty acyl-ACP thioesterase, a ketoacyl-ACP synthase, or a desaturase enzyme.

In another aspect, the present invention provides a triglyceride oil produced by a method as discussed above or herein. In various embodiments, any of the features discussed above or herein may be combined in any manner.

These and other aspects and embodiments of the invention are described and/or exemplified in the accompanying drawings, a brief description of which immediately follows, the detailed description of the invention, and in the examples. Any or all of the features discussed above and throughout the application can be combined in various embodiments of the present invention.

### BRIEF DESCRIPTION OF THE DRAWINGS

FIGS. 1a and 1b: FIG. 1a is the DSC heating curve of non-hydrogenated S8610 oil and FIG. 1b is the cooling curve of non-hydrogenated S8610 oil.

FIGS. 2a and 2B: FIG. 2a is the DSC heating curve of hydrogenated S8610 oil and FIG. 2b is the cooling curve of hydrogenated S8610 oil.

FIGS. 3a and 3b: FIG. 3a is the DSC heating curve of a distillate fraction of the hydrogenated S8610 oil and FIG. 3b is the cooling curve of residue fraction the hydrogenated S8610 oil.

### DETAILED DESCRIPTION OF THE INVENTION

### I. Definitions

An “allele” refers to a copy of a gene where an organism has multiple similar or identical gene copies, even if on the same chromosome. An allele may encode the same or similar protein.

“Ambient” pressure and temperature, as those terms are used herein, shall mean about 1 atmosphere and about 15-25° C., respectively, unless otherwise specified.

In connection with two fatty acids in a fatty acid profile, “balanced” shall mean that the two fatty acids are within a specified percentage of their mean area percent. Thus, for fatty acid a in x % abundance and fatty acid b in y % abundance, the fatty acids are “balanced to within z %” if |x−((x+y)/2)| and |y−((x+y)/2)| are ≤100(z).

“Asymmetric triglyceride” shall mean a triacylglyceride molecule in which the fatty acids at the sn-1 and the sn-3 position of the glycerol backbone are different.

A “cell oil” or “cell fat” shall mean a predominantly triglyceride oil obtained from an organism, where the oil has not undergone blending with another natural or synthetic oil, or fractionation so as to substantially alter the fatty acid profile of the triglyceride. In connection with an oil comprising triglycerides of a particular regiospecificity, the cell oil or cell fat has not been subjected to interesterification or other synthetic process to obtain that regiospecific triglyceride profile, rather the regiospecificity is produced naturally, by a cell or population of cells. For a cell oil produced by a cell, the sterol profile of oil is generally determined by the sterols produced by the cell, not by artificial reconstitution of the oil by adding sterols in order to mimic the cell oil. In connection with a cell oil or cell fat, and as used generally throughout the present disclosure, the terms oil and fat are used interchangeably, except where otherwise noted. Thus, an “oil” or a “fat” can be liquid, solid, or partially solid at room temperature, depending on the makeup of the substance and other conditions. Here, the term “fractionation” means removing material from the oil in a way that changes its fatty acid profile relative to the profile produced by the organism, however accomplished. The terms “cell oil” and “cell fat” encompass such oils obtained from an organism, where the oil has undergone minimal processing, including refining, bleaching and/or degumming, which does not substantially change its triglyceride profile. A cell oil can also be a “noninteresterified cell oil”, which means that the cell oil has not undergone a process in which fatty acids have been redistributed in their acyl linkages to glycerol and remain essentially in the same configuration as when recovered from the organism.

“Exogenous gene” shall mean a nucleic acid that codes for the expression of an RNA and/or protein that has been introduced into a cell (e.g. by transformation/transfection), and is also referred to as a “transgene”. A cell comprising an exogenous gene may be referred to as a recombinant cell, into which additional exogenous gene(s) may be introduced. The exogenous gene may be from a different species (and so heterologous), or from the same species (and so homologous), relative to the cell being transformed. Thus, an exogenous gene can include a homologous gene that occupies a different location in the genome of the cell or is under different control, relative to the endogenous copy of the gene. An exogenous gene may be present in more than one copy in the cell. An exogenous gene may be maintained in a cell as an insertion into the genome (nuclear or plastid) or as an episomal molecule.

“FADc”, also referred to as “FAD2” is a gene encoding a delta-12 fatty acid desaturase.

“Fatty acids” shall mean free fatty acids, fatty acid salts, or fatty acyl moieties in a glycerolipid. It will be understood that fatty acyl groups of glycerolipids can be described in terms of the carboxylic acid or anion of a carboxylic acid that is produced when the triglyceride is hydrolyzed or saponified.

“Fixed carbon source” is a molecule(s) containing carbon, typically an organic molecule that is present at ambient temperature and pressure in solid or liquid form in a culture media that can be utilized by a microorganism cultured therein. Accordingly, carbon dioxide is not a fixed carbon source.

“In operable linkage” is a functional linkage between two nucleic acid sequences, such a control sequence (typically a promoter) and the linked sequence (typically a sequence that encodes a protein, also called a coding sequence). A promoter is in operable linkage with an exogenous gene if it can mediate transcription of the gene.

“Microalgae” are eukaryotic microbial organisms that contain a chloroplast or other plastid, and optionally that is capable of performing photosynthesis, or a prokaryotic microbial organism capable of performing photosynthesis. Microalgae include obligate photoautotrophs, which cannot metabolize a fixed carbon source as energy, as well as heterotrophs, which can live solely off of a fixed carbon source.

Microalgae include unicellular organisms that separate from sister cells shortly after cell division, such as Chlamydomonas, as well as microbes such as, for example, Volvox, which is a simple multicellular photosynthetic microbe of two distinct cell types. Microalgae include cells such as Chlorella, Dunaliella, and Prototheca. Microalgae also include other microbial photosynthetic organisms that exhibit cell-cell adhesion, such as Agmenellum, Anabaena, and Pyrobotrys. Microalgae also include obligate heterotrophic microorganisms that have lost the ability to perform photosynthesis, such as certain dinoflagellate algae species and species of the genus Prototheca.

In connection with fatty acid length, “mid-chain” shall mean C8 to C16 fatty acids.

In connection with a recombinant cell, the term “knockdown” refers to a gene that has been partially suppressed (e.g., by about 1-95%) in terms of the production or activity of a protein encoded by the gene.

Also, in connection with a recombinant cell, the term “knockout” refers to a gene that has been completely or nearly completely (e.g., >95%) suppressed in terms of the production or activity of a protein encoded by the gene. Knockouts can be prepared by homologous recombination of a noncoding sequence into a coding sequence, gene deletion, mutation or other method.

An “oleaginous” cell is a cell capable of producing at least 20% lipid by dry cell weight, naturally or through recombinant or classical strain improvement. An “oleaginous microbe” or “oleaginous microorganism” is a microbe, including a microalga that is oleaginous (especially eukaryotic microalgae that store lipid). An oleaginous cell also encompasses a cell that has had some or all of its lipid or other content removed, and both live and dead cells.

An “ordered oil” or “ordered fat” is one that forms crystals that are primarily of a given polymorphic structure. For example, an ordered oil or ordered fat can have crystals that are greater than 50%, 60%, 70%, 80%, or 90% of the β or β′ polymorphic form.

In connection with a cell oil, a “profile” is the distribution of particular species or triglycerides or fatty acyl groups within the oil. A “fatty acid profile” is the distribution of fatty acyl groups in the triglycerides of the oil without reference to attachment to a glycerol backbone. Fatty acid profiles are typically determined by conversion to a fatty acid methyl ester (FAME), followed by gas chromatography (GC) analysis with flame ionization detection (FID), as in Example 1. The fatty acid profile can be expressed as one or more percent of a fatty acid in the total fatty acid signal determined from the area under the curve for that fatty acid. FAME-GC-FID measurement approximate weight percentages of the fatty acids. A “sn-2 profile” is the distribution of fatty acids found at the sn-2 position of the triacylglycerides in the oil. A “regiospecific profile” is the distribution of triglycerides with reference to the positioning of acyl group attachment to the glycerol backbone without reference to stereospecificity. In other words, a regiospecific profile describes acyl group attachment at sn-1/3 vs. sn-2. Thus, in a regiospecific profile, POS (palmitate-oleate-stearate) and SOP (stearate-oleate-palmitate) are treated identically. A “stereospecific profile” describes the attachment of acyl groups at sn-1, sn-2 and sn-3. Unless otherwise indicated, triglycerides such as SOP and POS are to be considered equivalent. A “TAG profile” is the distribution of fatty acids found in the triglycerides with reference to connection to the glycerol backbone, but without reference to the regiospecific nature of the connections. Thus, in a TAG profile, the percent of SSO in the oil is the sum of SSO and SOS, while in a regiospecific profile, the percent of SSO is calculated without inclusion of SOS species in the oil. In contrast to the weight percentages of the FAME-GC-FID analysis, triglyceride percentages are typically given as mole percentages; that is the percent of a given TAG molecule in a TAG mixture.

The term “percent sequence identity,” in the context of two or more amino acid or nucleic acid sequences, refers to two or more sequences or subsequences that are the same or have a specified percentage of amino acid residues or nucleotides that are the same, when compared and aligned for maximum correspondence, as measured using a sequence comparison algorithm or by visual inspection. For sequence comparison to determine percent nucleotide or amino acid identity, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters. Optimal alignment of sequences for comparison can be conducted using the NCBI BLAST software (ncbi.nlm.nih.gov/BLAST/) set to default parameters. For example, to compare two nucleic acid sequences, one may use blastn with the “BLAST 2 Sequences” tool Version 2.0.12 (Apr. 21, 2000) set at the following default parameters: Matrix: BLOSUM62; Reward for match: 1; Penalty for mismatch: −2; Open Gap: 5 and Extension Gap: 2 penalties; Gap×drop-off: 50; Expect: 10; Word Size: 11; Filter: on. For a pairwise comparison of two amino acid sequences, one may use the “BLAST 2 Sequences” tool Version 2.0.12 (Apr. 21, 2000) with blastp set, for example, at the following default parameters: Matrix: BLOSUM62; Open Gap: 11 and Extension Gap: 1 penalties; Gap×drop-off 50; Expect: 10; Word Size: 3; Filter: on.

“Recombinant” is a cell, nucleic acid, protein or vector that has been modified due to the introduction of an exogenous nucleic acid or the alteration of a native nucleic acid. Thus, e.g., recombinant cells can express genes that are not found within the native (non-recombinant) form of the cell or express native genes differently than those genes are expressed by a non-recombinant cell. Recombinant cells can, without limitation, include recombinant nucleic acids that encode for a gene product or for suppression elements such as mutations, knockouts, antisense, interfering RNA (RNAi) or dsRNA that reduce the levels of active gene product in a cell. A “recombinant nucleic acid” is a nucleic acid originally formed in vitro, in general, by the manipulation of nucleic acid, e.g., using polymerases, ligases, exonucleases, and endonucleases, using chemical synthesis, or otherwise is in a form not normally found in nature. Recombinant nucleic acids may be produced, for example, to place two or more nucleic acids in operable linkage. Thus, an isolated nucleic acid or an expression vector formed in vitro by ligating DNA molecules that are not normally joined in nature, are both considered recombinant for the purposes of this invention. Once a recombinant nucleic acid is made and introduced into a host cell or organism, it may replicate using the in vivo cellular machinery of the host cell; however, such nucleic acids, once produced recombinantly, although subsequently replicated intracellularly, are still considered recombinant for purposes of this invention. Similarly, a “recombinant protein” is a protein made using recombinant techniques, i.e., through the expression of a recombinant nucleic acid.

The terms “triglyceride”, “triacylglyceride” and “TAG” are used interchangeably as is known in the art.

### II. General

Illustrative embodiments of the present invention feature oleaginous cells that produce altered fatty acid profiles and/or altered regiospecific distribution of fatty acids in glycerolipids, and products produced from the cells. Examples of oleaginous cells include microbial cells having a type II fatty acid biosynthetic pathway, including plastidic oleaginous cells such as those of oleaginous algae and, where applicable, oil producing cells of higher plants including but not limited to commercial oilseed crops such as soy, corn, rapeseed/canola, cotton, flax, sunflower, safflower and peanut. Other specific examples of cells include heterotrophic or obligate heterotrophic microalgae of the phylum Chlorophtya, the class Trebouxiophytae, the order Chlorellales, or the family Chlorellacae. Examples of oleaginous microalgae and method of cultivation are also provided in Published PCT Patent Applications WO2008/151149, WO2010/063032, WO2010/063031, WO2011/150410, and WO2011/150411, including species of Chlorella and Prototheca, a genus comprising obligate heterotrophs. The oleaginous cells can be, for example, capable of producing 25, 30, 40, 50, 60, 70, 80, 85, or about 90% oil by cell weight, ±5%. Optionally, the oils produced can be low in highly unsaturated fatty acids such as DHA or EPA fatty acids. For example, the oils can comprise less than 5%, 2%, or 1% DHA and/or EPA. The above-mentioned publications also disclose methods for cultivating such cells and extracting oil, especially from microalgal cells; such methods are applicable to the cells disclosed herein and incorporated by reference for these teachings. When microalgal cells are used they can be cultivated autotrophically (unless an obligate heterotroph) or in the dark using a sugar (e.g., glucose, fructose and/or sucrose) In any of the embodiments described herein, the cells can be heterotrophic cells comprising an exogenous invertase gene so as to allow the cells to produce oil from a sucrose feedstock. Alternately, or in addition, the cells can metabolize xylose from cellulosic feedstocks. For example, the cells can be genetically engineered to express one or more xylose metabolism genes such as those encoding an active xylose transporter, a xylulose-5-phosphate transporter, a xylose isomerase, a xylulokinase, a xylitol dehydrogenase and a xylose reductase. See WO2012/154626, “Genetically Engineered Microorganisms that Metabolize Xylose”, published Nov. 15, 2012, including disclosure of genetically engineered Prototheca strains that utilize xylose.

The oleaginous cells may, optionally, be cultivated in a bioreactor/fermenter. For example, heterotrophic oleaginous microalgal cells can be cultivated on a sugar-containing nutrient broth. Optionally, cultivation can proceed in two stages: a seed stage and a lipid-production stage. In the seed stage, the number of cells is increased from s starter culture. Thus, the seeds stage typically includes a nutrient rich, nitrogen replete, media designed to encourage rapid cell division. After the seeds stage, the cells may be fed sugar under nutrient-limiting (e.g. nitrogen sparse) conditions so that the sugar will be converted into triglycerides. For example, the rate of cell division in the lipid-production stage can be decreased by 50%, 80% or more relative to the seed stage. Additionally, variation in the media between the seed stage and the lipid-production stage can induce the recombinant cell to express different lipid-synthesis genes and thereby alter the triglycerides being produced. For example, as discussed below, nitrogen and/or pH sensitive promoters can be placed in front of endogenous or exogenous genes. This is especially useful when an oil is to be produced in the lipid-production phase that does not support optimal growth of the cells in the seed stage. In an example below, a cell has a fatty acid desaturase with a pH sensitive promoter so than an oil that is low in linoleic acid is produced in the lipid production stage while an oil that has adequate linoleic acid for cell division is produced during the seed stage. The resulting low linoleic oil has exceptional oxidative stability.

The oleaginous cells express one or more exogenous genes encoding fatty acid biosynthesis enzymes. As a result, some embodiments feature cell oils that were not obtainable from a non-plant or non-seed oil, or not obtainable at all.

The oleaginous cells (optionally microalgal cells) can be improved via classical strain improvement techniques such as UV and/or chemical mutagenesis followed by screening or selection under environmental conditions, including selection on a chemical or biochemical toxin. For example the cells can be selected on a fatty acid synthesis inhibitor, a sugar metabolism inhibitor, or an herbicide. As a result of the selection, strains can be obtained with increased yield on sugar, increased oil production (e.g., as a percent of cell volume, dry weight, or liter of cell culture), or improved fatty acid or TAG profile.

For example, the cells can be selected on one or more of 1,2-Cyclohexanedione; 19-Norethindone acetate; 2,2-dichloropropionic acid; 2,4,5-trichlorophenoxyacetic acid; 2,4,5-trichlorophenoxyacetic acid, methyl ester; 2,4-dichlorophenoxyacetic acid; 2,4-dichlorophenoxyacetic acid, butyl ester; 2,4-dichlorophenoxyacetic acid, isooctyl ester; 2,4-dichlorophenoxyacetic acid, methyl ester; 2,4-dichlorophenoxybutyric acid; 2,4-dichlorophenoxybutyric acid, methyl ester; 2,6-dichlorobenzonitrile; 2-deoxyglucose; 5-Tetradecyloxy-w-furoic acid; A-922500; acetochlor; alachlor; ametryn; amphotericin; atrazine; benfluralin; bensulide; bentazon; bromacil; bromoxynil; Cafenstrole; carbonyl cyanide m-chlorophenyl hydrazone (CCCP); carbonyl cyanide-p-trifluoromethoxyphenylhydrazone (FCCP); cerulenin; chlorpropham; chlorsulfuron; clofibric acid; clopyralid; colchicine; cycloate; cyclohexamide; C75; DACTHAL (dimethyl tetrachloroterephthalate); dicamba; dichloroprop ((R)-2-(2,4-dichlorophenoxy)propanoic acid); Diflufenican; dihyrojasmonic acid, methyl ester; diquat; diuron; dimethylsulfoxide; Epigallocatechin gallate (EGCG); endothall; ethalfluralin; ethanol; ethofumesate; Fenoxaprop-p-ethyl; Fluazifop-p-Butyl; fluometuron; fomasefen; foramsulfuron; gibberellic acid; glufosinate ammonium; glyphosate; haloxyfop; hexazinone; imazaquin; isoxaben; Lipase inhibitor THL ((−)-Tetrahydrolipstatin); malonic acid; MCPA (2-methyl-4-chlorophenoxyacetic acid); MCPB (4-(4-chloro-o-tolyloxy)butyric acid); mesotrione; methyl dihydrojasmonate; metolachlor; metribuzin; Mildronate; molinate; naptalam; norharman; orlistat; oxadiazon; oxyfluorfen; paraquat; pendimethalin; pentachlorophenol; PF-04620110; phenethyl alcohol; phenmedipham; picloram; Platencin; Platensimycin; prometon; prometryn; pronamide; propachlor; propanil; propazine; pyrazon; Quizalofop-p-ethyl; s-ethyl dipropylthiocarbamate (EPTC); s,s,s-tributylphosphorotrithioate; salicylhydroxamic acid; sesamol; siduron; sodium methane arsenate; simazine; T-863 (DGAT inhibitor); tebuthiuron; terbacil; thiobencarb; tralkoxydim; triallate; triclopyr; triclosan; trifluralin; and vulpinic acid.

The oleaginous cells produce a storage oil, which is primarily triacylglyceride and may be stored in storage bodies of the cell. A raw oil may be obtained from the cells by disrupting the cells and isolating the oil. The raw oil may comprise sterols produced by the cells. WO2008/151149, WO2010/063032, WO2011/150410, and WO2011/1504 disclose heterotrophic cultivation and oil isolation techniques for oleaginous microalgae. For example, oil may be obtained by providing or cultivating, drying and pressing the cells. The oils produced may be refined, bleached and deodorized (RBD) as known in the art or as described in WO2010/120939. The raw or RBD oils may be used in a variety of food, chemical, and industrial products or processes. Even after such processing, the oil may retain a sterol profile characteristic of the source. Microalgal sterol profiles are disclosed below. See especially Section XII of this patent application. After recovery of the oil, a valuable residual biomass remains. Uses for the residual biomass include the production of paper, plastics, absorbents, adsorbents, drilling fluids, as animal feed, for human nutrition, or for fertilizer.

Where a fatty acid profile of a triglyceride (also referred to as a “triacylglyceride” or “TAG”) cell oil is given here, it will be understood that this refers to a nonfractionated sample of the storage oil extracted from the cell analyzed under conditions in which phospholipids have been removed or with an analysis method that is substantially insensitive to the fatty acids of the phospholipids (e.g. using chromatography and mass spectrometry). The oil may be subjected to an RBD process to remove phospholipids, free fatty acids and odors yet have only minor or negligible changes to the fatty acid profile of the triglycerides in the oil. Because the cells are oleaginous, in some cases the storage oil will constitute the bulk of all the TAGs in the cell. Examples 1, 2, and 3 below give analytical methods for determining TAG fatty acid composition and regiospecific structure.

Broadly categorized, certain embodiments of the invention include (i) auxotrophs of particular fatty acids; (ii) cells that produce oils having low concentrations of polyunsaturated fatty acids, including cells that are auxotrophic for unsaturated fatty acids; (iii) cells producing oils having high concentrations of particular fatty acids due to expression of one or more exogenous genes encoding enzymes that transfer fatty acids to glycerol or a glycerol ester; (iv) cells producing regiospecific oils, and (v) other inventions related to producing cell oils with altered profiles. The embodiments also encompass the oils made by such cells, the residual biomass from such cells after oil extraction, oleochemicals, fuels and food products made from the oils and methods of cultivating the cells.

In any of the embodiments below, the cells used are optionally cells having a type II fatty acid biosynthetic pathway such as microalgal cells including heterotrophic or obligate heterotrophic microalgal cells, including cells classified as Chlorophyta, Trebouxiophyceae, Chlorellales, Chlorellaceae, or Chlorophyceae, or cells engineered to have a type II fatty acid biosynthetic pathway using the tools of synthetic biology (i.e., transplanting the genetic machinery for a type II fatty acid biosynthesis into an organism lacking such a pathway). Use of a host cell with a type II pathway avoids the potential for non-interaction between an exogenous acyl-ACP thioesterase or other ACP-binding enzyme and the multienzyme complex of type I cellular machinery. In specific embodiments, the cell is of the species Prototheca moriformis, Prototheca krugani, Prototheca stagnora or Prototheca zopfii or has a 23S rRNA sequence with at least 65, 70, 75, 80, 85, 90 or 95% nucleotide identity SEQ ID NO: 1. By cultivating in the dark or using an obligate heterotroph, the cell oil produced can be low in chlorophyll or other colorants. For example, the cell oil can have less than 100, 50, 10, 5, 1, 0.0.5 ppm of chlorophyll without substantial purification.

In specific embodiments and examples discussed below, one or more fatty acid synthesis genes (e.g., encoding an acyl-ACP thioesterase, a keto-acyl ACP synthase, a stearoyl ACP desaturase, or others described herein) is incorporated into a microalga. It has been found that for certain microalga, a plant fatty acid synthesis gene product is functional in the absence of the corresponding plant acyl carrier protein (ACP), even when the gene product is an enzyme, such as an acyl-ACP thioesterase, that requires binding of ACP to function. Thus, optionally, the microalgal cells can utilize such genes to make a desired oil without co-expression of the plant ACP gene. Examples of cells engineered to express various enzymes can be found in, for example, WO 2015/051319.

For the various embodiments of recombinant cells comprising exogenous genes or combinations of genes, it is contemplated that substitution of those genes with genes having 60, 70, 80, 85, 90, 91, 92, 93, 94, 95, 96, 97, 98, or 99% nucleic acid sequence identity can give similar results, as can substitution of genes encoding proteins having 60, 70, 80, 85, 90, 91, 92, 93, 94, 95, 95.5, 96, 96.5, 97, 97.5, 98, 98.5, 99 or 99.5% amino acid sequence identity. Likewise, for novel regulatory elements, it is contemplated that substitution of those nucleic acids with nucleic acids having 60, 70, 80, 85, 90, 91, 92, 93, 94, 95, 96, 97, 98, or 99% nucleic acid can be efficacious. In the various embodiments, it will be understood that sequences that are not necessary for function (e.g. FLAG® tags or inserted restriction sites) can often be omitted in use or ignored in comparing genes, proteins and variants.

Although discovered using or exemplified with microalgae, the novel genes and gene combinations reported here can be used in higher plants using techniques that are well known in the art. For example, the use of exogenous lipid metabolism genes in higher plants is described in U.S. Pat. Nos. 6,028,247, 5,850,022, 5,639,790, 5,455,167, 5,512,482, and 5,298,421 disclose higher plants with exogenous acyl-ACP thioesterases. FAD2 suppression in higher plants is taught in WO 2013112578, and WO 2008006171.

### III. Fatty Acid Auxotrophs/Reducing Fatty Acid Levels to Growth Inhibitory Conditions During an Oil Production Phase

In an embodiment, the cell is genetically engineered so that all alleles of a lipid pathway gene are knocked out. Alternately, the amount or activity of the gene products of the alleles is knocked down so as to require supplementation with fatty acids. A first transformation construct can be generated bearing donor sequences homologous to one or more of the alleles of the gene. This first transformation construct may be introduced and selection methods followed to obtain an isolated strain characterized by one or more allelic disruptions. Alternatively, a first strain may be created that is engineered to express a selectable marker from an insertion into a first allele, thereby inactivating the first allele. This strain may be used as the host for still further genetic engineering to knockout or knockdown the remaining allele(s) of the lipid pathway gene (e.g., using a second selectable marker to disrupt a second allele). Complementation of the endogenous gene can be achieved through engineered expression of an additional transformation construct bearing the endogenous gene whose activity was originally ablated, or through the expression of a suitable heterologous gene. The expression of the complementing gene can either be regulated constitutively or through regulatable control, thereby allowing for tuning of expression to the desired level so as to permit growth or create an auxotrophic condition at will. In an embodiment, a population of the fatty acid auxotroph cells are used to screen or select for complementing genes; e.g., by transformation with particular gene candidates for exogenous fatty acid synthesis enzymes, or a nucleic acid library believed to contain such candidates.

Knockout of all alleles of the desired gene and complementation of the knocked-out gene need not be carried out sequentially. The disruption of an endogenous gene of interest and its complementation either by constitutive or inducible expression of a suitable complementing gene can be carried out in several ways. In one method, this can be achieved by co-transformation of suitable constructs, one disrupting the gene of interest and the second providing complementation at a suitable, alternative locus. In another method, ablation of the target gene can be effected through the direct replacement of the target gene by a suitable gene under control of an inducible promoter (“promoter hijacking”). In this way, expression of the targeted gene is now put under the control of a regulatable promoter. An additional approach is to replace the endogenous regulatory elements of a gene with an exogenous, inducible gene expression system. Under such a regime, the gene of interest can now be turned on or off depending upon the particular needs. A still further method is to create a first strain to express an exogenous gene capable of complementing the gene of interest, then to knockout out or knockdown all alleles of the gene of interest in this first strain. The approach of multiple allelic knockdown or knockout and complementation with exogenous genes may be used to alter the fatty acid profile, regiospecific profile, sn-2 profile, or the TAG profile of the engineered cell.

Where a regulatable promoter is used, the promoter can be pH-sensitive (e.g., amt03), nitrogen and pH sensitive (e.g., amt03), or nitrogen sensitive but pH-insensitive (see, e.g., WO 2015/051319) or variants thereof comprising at least 60, 65, 70, 75, 80, 85, 90, 95, 96, 97, 98 or 99% sequence identity to any of the aforementioned promoters. In connection with a promoter, pH-insensitive means that the promoter is less sensitive than the amt03 promoter when environmental conditions are shifter from pH 6.8 to 5.0 (e.g., at least 5, 10, 15, or 20% less relative change in activity upon the pH-shift as compared to an equivalent cell with amt03 as the promoter).

In a specific embodiment, the recombinant cell comprises nucleic acids operable to reduce the activity of an endogenous acyl-ACP thioesterase; for example a FatA or FatB acyl-ACP thioesterase having a preference for hydrolyzing fatty acyl-ACP chains of length C18 (e.g., stearate (C18:0) or oleate (C18:1), or C8:0-C16:0 fatty acids. The activity of an endogenous acyl-ACP thioesterase may be reduced by knockout or knockdown approaches. Knockdown may be achieved, for example, through the use of one or more RNA hairpin constructs, by promoter hijacking (substitution of a lower activity or inducible promoter for the native promoter of an endogenous gene), or by a gene knockout combined with introduction of a similar or identical gene under the control of an inducible promoter. WO2015/051319 describes the engineering of a Prototheca strain in which two alleles of the endogenous fatty acyl-ACP thioesterase (FATA1) have been knocked out. The activity of the Prototheca moriformis FATA1 was complemented by the expression of an exogenous FatA or FatB acyl-ACP thioesterase. WO2015/051319 details the use of RNA hairpin constructs to reduce the expression of FATA in Prototheca, which resulted in an altered fatty acid profile having less palmitic acid and more oleic acid.

Accordingly, oleaginous cells, including those of organisms with a type II fatty acid biosynthetic pathway can have knockouts or knockdowns of acyl-ACP thioesterase-encoding alleles to such a degree as to eliminate or severely limit viability of the cells in the absence of fatty acid supplementation or genetic complementations. These strains can be used to select for transformants expressing acyl-ACP-thioesterase transgenes. Alternately, or in addition, the strains can be used to completely transplant exogenous acyl-ACP-thioesterases to give dramatically different fatty acid profiles of cell oils produced by such cells. For example, FATA expression can be completely or nearly completely eliminated and replaced with FATB genes that produce mid-chain fatty acids. Alternately, an organism with an endogenous FatA gene having specificity for palmitic acid (C16) relative to stearic or oleic acid (C18) can be replaced with an exogenous FatA gene having a greater relative specificity for stearic acid (C18:0) or replaced with an exogenous FatA gene having a greater relative specificity for oleic acid (C18:1). In certain specific embodiments, these transformants with double knockouts of an endogenous acyl-ACP thioesterase produce cell oils with more than 50, 60, 70, 80, or 90% caprylic, capric, lauric, myristic, or palmitic acid, or total fatty acids of chain length less than 18 carbons. Such cells may require supplementation with longer chain fatty acids such as stearic or oleic acid or switching of environmental conditions between growth permissive and restrictive states in the case of an inducible promoter regulating a FatA gene.

In an embodiment the oleaginous cells are cultured (e.g., in a bioreactor). The cells are fully auxotrophic or partially auxotrophic (i.e., lethality or synthetic sickness) with respect to one or more types of fatty acid. The cells are cultured with supplementation of the fatty acid(s) so as to increase the cell number, then allowing the cells to accumulate oil (e.g. to at least 40% by dry cell weight). Alternatively, the cells comprise a regulatable fatty acid synthesis gene that can be switched in activity based on environmental conditions and the environmental conditions during a first, cell division, phase favor production of the fatty acid and the environmental conditions during a second, oil accumulation, phase disfavor production of the fatty acid. In the case of an inducible gene, the regulation of the inducible gene can be mediated, without limitation, via environmental pH (for example, by using the AMT3 promoter as described in, e.g., WO2015/051319).

As a result of applying either of these supplementation or regulation methods, a cell oil may be obtained from the cell that has low amounts of one or more fatty acids essential for optimal cell propagation. Specific examples of oils that can be obtained include those low in stearic, linoleic and/or linolenic acids.

These cells and methods are illustrated in connection with low polyunsaturated oils in the section immediately below. Specific examples can be found in, e.g., WO2015/051319.

Likewise, fatty acid auxotrophs can be made in other fatty acid synthesis genes including those encoding a SAD, FAD, KASIII, KASI, KASII, KCS, elongase, GPAT, LPAAT, DGAT or AGPAT or PAP. These auxotrophs can also be used to select for complement genes or to eliminate native expression of these genes in favor of desired exogenous genes in order to alter the fatty acid profile, regiospecific profile, or TAG profile of cell oils produced by oleaginous cells.

Accordingly, in an embodiment of the invention, there is a method for producing an oil/fat. The method comprises cultivating a recombinant oleaginous cell in a growth phase under a first set of conditions that is permissive to cell division so as to increase the number of cells due to the presence of a fatty acid, cultivating the cell in an oil production phase under a second set of conditions that is restrictive to cell division but permissive to production of an oil that is depleted in the fatty acid, and extracting the oil from the cell, wherein the cell has a mutation or exogenous nucleic acids operable to suppress the activity of a fatty acid synthesis enzyme, the enzyme optionally being a stearoyl-ACP desaturase, delta 12 fatty acid desaturase, or a ketoacyl-ACP synthase. The oil produced by the cell can be depleted in the fatty acid by at least 50, 60, 70, 80, or 90%. The cell can be cultivated heterotrophically. The cell can be a microalgal cell cultivated heterotrophically or autotrophically and may produce at least 40, 50, 60, 70, 80, or 90% oil by dry cell weight.

### IV. Low Polyunsaturated Cell Oils

In an embodiment of the present invention, the cell oil produced by the cell has very low levels of polyunsaturated fatty acids. As a result, the cell oil can have improved stability, including oxidative stability. The cell oil can be a liquid or solid at room temperature, or a blend of liquid and solid oils, including the regiospecific or stereospecific oils, high stearate oils, or high mid-chain oils described infra. Oxidative stability can be measured by the Rancimat method using the AOCS Cd 12b-92 standard test at a defined temperature. For example, the OSI (oxidative stability index) test may be run at temperatures between 110° C. and 140° C. The oil is produced by cultivating cells (e.g., any of the plastidic microbial cells mentioned above or elsewhere herein) that are genetically engineered to reduce the activity of one or more fatty acid desaturase. For example, the cells may be genetically engineered to reduce the activity of one or more fatty acyl 412 desaturase(s) responsible for converting oleic acid (18:1) into linoleic acid (18:2) and/or one or more fatty acyl 415 desaturase(s) responsible for converting linoleic acid (18:2) into linolenic acid (18:3). Various methods may be used to inhibit the desaturase including knockout or mutation of one or more alleles of the gene encoding the desaturase in the coding or regulatory regions, inhibition of RNA transcription, or translation of the enzyme, including RNAi, siRNA, miRNA, dsRNA, antisense, and hairpin RNA techniques. Other techniques known in the art can also be used including introducing an exogenous gene that produces an inhibitory protein or other substance that is specific for the desaturase. In specific examples, a knockout of one fatty acyl Δ12 desaturase allele is combined with RNA-level inhibition of a second allele.

In a specific embodiment, fatty acid desaturase (e.g., 412 fatty acid desaturase) activity in the cell is reduced to such a degree that the cell is unable to be cultivated or is difficult to cultivate (e.g., the cell division rate is decreased more than 10, 20, 30, 40, 50, 60, 70, 80, 90, 95, 97 or 99%). Achieving such conditions may involve knockout, or effective suppression of the activity of multiple gene copies (e.g. 2, 3, 4 or more) of the desaturase or their gene products. A specific embodiment includes the cultivation in cell culture of a full or partial fatty acid auxotroph with supplementation of the fatty acid or a mixture of fatty acids so as to increase the cell number, then allowing the cells to accumulate oil (e.g. to at least 40% by cell weight). Alternatively, the cells comprise a regulatable fatty acid synthesis gene that can be switched in activity. For example, the regulation can be based on environmental conditions and the environmental conditions during a first, cell division, phase favor production of the fatty acid and the environmental conditions during a second, oil accumulation, phase disfavor production of the oil. For example, culture media pH and/or nitrogen levels can be used as an environmental control to switch expression of a lipid pathway gene to produce a state of high or low synthetic enzyme activity. Examples of such cells are described in, e.g., WO2015/051319.

In a specific embodiment, a cell is cultivated using a modulation of linoleic acid levels within the cell. In particular, the cell oil is produced by cultivating the cells under a first condition that is permissive to an increase in cell number due to the presence of linoleic acid and then cultivating the cells under a second condition that is characterized by linoleic acid starvation and thus is inhibitory to cell division, yet permissive of oil accumulation. For example, a seed culture of the cells may be produced in the presence of linoleic acid added to the culture medium. For example, the addition of linoleic acid to 0.25 g/L in the seed culture of a Prototheca strain deficient in linoleic acid production due to ablation of two alleles of a fatty acyl 412 desaturase (i.e., a linoleic auxotroph) was sufficient to support cell division to a level comparable to that of wild type cells. Optionally, the linoleic acid can then be consumed by the cells, or otherwise removed or diluted. The cells are then switched into an oil producing phase (e.g., supplying sugar under nitrogen limiting conditions such as described in WO2010/063032). Surprisingly, oil production has been found to occur even in the absence of linoleic acid production or supplementation, as demonstrated in the obligate heterotroph oleaginous microalgae Prototheca but generally applicable to other oleaginous microalgae, microorganisms, or even multicellular organisms (e.g., cultured plant cells). Under these conditions, the oil content of the cell can increase to about 10, 20, 30, 40, 50, 60, 70, 80, 90%, or more by dry cell weight, while the oil produced can have polyunsaturated fatty acid (e.g.; linoleic+linolenic) profile with 5%, 4%, 3%, 2%, 1%, 0.5%, 0.3%, 0.2%, 0.1%, 0.05% or less, as a percent of total triacylglycerol fatty acids in the oil. For example, the oil content of the cell can be 50% or more by dry cell weight and the triglyceride of the oil produced less than 3% polyunsaturated fatty acids.

These oils can also be produced without the need (or reduced need) to supplement the culture with linoleic acid by using cell machinery to produce the linoleic acid during the cell division phase, but less or no linoleic acid in the lipid production phase. The linoleic-producing cell machinery may be regulatable so as to produce substantially less linoleic acid during the oil producing phase. The regulation may be via modulation of transcription of the desaturase gene(s) or modulation or modulation of production of an inhibitor substance (e.g., regulated production of hairpin RNA/RNAi). For example, the majority, and preferably all, of the fatty acid Δ12 desaturase activity can be placed under a regulatable promoter regulated to express the desaturase in the cell division phase, but to be reduced or turned off during the oil accumulation phase. The regulation can be linked to a cell culture condition such as pH, and/or nitrogen level, as described in the examples herein, or other environmental condition. In practice, the condition may be manipulated by adding or removing a substance (e.g., protons via addition of acid or base) or by allowing the cells to consume a substance (e.g., nitrogen-supplying nutrients) to effect the desired switch in regulation of the desaturase activity.

Other genetic or non-genetic methods for regulating the desaturase activity can also be used. For example, an inhibitor of the desaturase can be added to the culture medium in a manner that is effective to inhibit polyunsaturated fatty acids from being produced during the oil production phase.

Accordingly, in a specific embodiment of the invention, there is a method comprising providing a recombinant cell having a regulatable delta 12 fatty acid desaturase gene, under control of a recombinant regulatory element via an environmental condition. The cell is cultivated under conditions that favor cell multiplication. Upon reaching a given cell density, the cell media is altered to switch the cells to lipid production mode by nutrient limitation (e.g. reduction of available nitrogen). During the lipid production phase, the environmental condition is such that the activity of the delta 12 fatty acid desaturase is downregulated. The cells are then harvested and, optionally, the oil extracted. Due to the low level of delta 12 fatty acid desaturase during the lipid production phase, the oil has less polyunsaturated fatty acids and has improved oxidative stability. Optionally the cells are cultivated heterotrophically and optionally microalgal cells.

Using one or more of these desaturase regulation methods, it is possible to obtain a cell oil that it is believed has been previously unobtainable, especially in large scale cultivation in a bioreactor (e.g., more than 1000 L). The oil can have polyunsaturated fatty acid levels that are 5%, 4%, 3%, 2%, 1%, 0.5%, 0.3%, 0.2%, or less, as an area percent of total triacylglycerol fatty acids in the oil.

One consequence of having such low levels of polyunsaturates is that oils are exceptionally stable to oxidation. Indeed, in some cases the oils may be more stable than any previously known cell cell oil. In specific embodiments, the oil is stable, without added antioxidants, at 110° C. so that the inflection point in conductance is not yet reached by 10 hours, 15 hours, 20 hours, 30 hours, 40, hours, 50 hours, 60 hours, or 70 hours under conditions of the AOCS Cd 12b-92. Rancimat test, noting that for very stable oils, replenishment of water may be required in such a test due to evaporation that occurs with such long testing periods. For example the oil can have and OSI value of 40-50 hours or 41-46 hours at 110° C. without added antioxidants. When antioxidants (suitable for foods or otherwise) are added, the OSI value measured may be further increased. For example, with added tocopherol (100 ppm) and ascorbyl palmitate (500 ppm) or PANA and ascorbyl palmitate, such an oil can have an oxidative stability index (OSI value) at 110° C. in excess 100 or 200 hours, as measured by the Rancimat test. In another example, 1050 ppm of mixed tocopherols and 500 pm of ascorbyl palmitate are added to an oil comprising less than 1% linoleic acid or less than 1% linoleic+linolenic acids; as a result, the oil is stable at 110° C. for 1, 2, 3, 4, 5, 6, 7, 8, or 9, 10, 11, 12, 13, 14, 15, or 16, 20, 30, 40 or 50 days, 5 to 15 days, 6 to 14 days, 7 to 13 days, 8 to 12 days, 9 to 11 days, about 10 days, or about 20 days. The oil can also be stable at 130° C. for 1, 2, 3, 4, 5, 6, 7, 8, or 9, 10, 11, 12, 13, 14, 15, or 16, 20, 30, 40 or 50 days, 5 to 15 days, 6 to 14 days, 7 to 13 days, 8 to 12 days, 9 to 11 days, about 10 days, or about 20 days. In a specific example, such an oil was found to be stable for greater than 100 hours (about 128 hours as observed). In a further embodiment, the OSI value of the cell oil without added antioxidants at 120° C. is greater than 15 hours or 20 hours or is in the range of 10-15, 15-20, 20-25, or 25-50 hours, or 50-100 hours.

In an example, using these methods, the oil content of a microalgal cell is between 40 and about 85% by dry cell weight and the polyunsaturated fatty acids in the fatty acid profile of the oil is between 0.001% and 3% in the fatty acid profile of the oil and optionally yields a cell oil having an OSI induction time of at least 20 hours at 110° C. without the addition of antioxidants. In yet another example, there is a cell oil produced by RBD treatment of a cell oil from an oleaginous cell, the oil comprises between 0.001% and 2% polyunsaturated fatty acids and has an OSI induction time exceeding 30 hours at 110 C without the addition of antioxidants. In yet another example, there is a cell oil produced by RBD treatment of a cell oil from an oleaginous cell, the oil comprises between 0.001% and 1% polyunsaturated fatty acids and has an OSI induction time exceeding 30 hours at 110 C without the addition of antioxidants.

In another specific embodiment there is an oil with reduced polyunsaturate levels produced by the above-described methods. The oil is combined with antioxidants such as PANA and ascorbyl palmitate. For example, it was found that when such an oil was combined with 0.5% PANA and 500 ppm of ascorbyl palmitate the oil had an OSI value of about 5 days at 130° C. or 21 days at 110° C. These remarkable results suggest that not only is the oil exceptionally stable, but these two antioxidants are exceptionally potent stabilizers of triglyceride oils and the combination of these antioxidants may have general applicability including in producing stable biodegradable lubricants (e.g., jet engine lubricants). In specific embodiments, the genetic manipulation of fatty acyl 412 desaturase results in a 2 to 30, or 5 to 25, or 10 to 20 fold increase in OSI (e.g., at 110° C.) relative to a strain without the manipulation. The oil can be produced by suppressing desaturase activity in a cell, including as described above.

Antioxidants suitable for use with the oils of the present invention include alpha, delta, and gamma tocopherol (vitamin E), tocotrienol, ascorbic acid (vitamin C), glutathione, lipoic acid, uric acid, β-carotene, lycopene, lutein, retinol (vitamin A), ubiquinol (coenzyme Q), melatonin, resveratrol, flavonoids, rosemary extract, propyl gallate (PG), tertiary butylhydroquinone (TBHQ), butylated hydroxyanisole (BHA), and butylated hydroxytoluene (BHT), N,N′-di-2-butyl-1,4-phenylenediamine,2,6-di-tert-butyl-4-methylphenol, 2,4-dimethyl-6-tert-butylphenol, 2,4-dimethyl-6-tert-butylphenol, 2,4-dimethyl-6-tert-butylphenol, 2,6-di-tert-butyl-4-methylphenol, 2,6-di-tert-butylphenol, and phenyl-alpha-naphthylamine (PANA).

In addition to the desaturase modifications, in a related embodiment other genetic modifications may be made to further tailor the properties of the oil, as described throughout, including introduction or substitution of acyl-ACP thioesterases having altered chain length specificity and/or overexpression of an endogenous or exogenous gene encoding a KAS, SAD, LPAAT, or DGAT gene. For example, a strain that produces elevated oleic levels may also produce low levels of polyunsaturates. Such genetic modifications can include increasing the activity of stearoyl-ACP desaturase (SAD) by introducing an exogenous SAD gene, increasing elongase activity by introducing an exogenous KASII gene, and/or knocking down or knocking out a FATA gene.

In a specific embodiment, a high oleic cell oil with low polyunsaturates may be produced. For example, the oil may have a fatty acid profile with greater than 60, 70, 80, 90, or 95% oleic acid and less than 5, 4, 3, 2, or 1% polyunsaturates. In related embodiments, a cell oil is produced by a cell having recombinant nucleic acids operable to decrease fatty acid 412 desaturase activity and optionally fatty acid 415 desaturase so as to produce an oil having less than or equal to 3% polyunsaturated fatty acids with greater than 60% oleic acid, less than 2% polyunsaturated fatty acids and greater than 70% oleic acid, less than 1% polyunsaturated fatty acids and greater than 80% oleic acid, or less than 0.5% polyunsaturated fatty acids and greater than 90% oleic acid. It has been found that one way to increase oleic acid is to use recombinant nucleic acids operable to decrease expression of a FATA acyl-ACP thioesterase and optionally overexpress a KAS II gene; such a cell can produce an oil with greater than or equal to 75% oleic acid. Alternately, overexpression of KASII can be used without the FATA knockout or knockdown. Oleic acid levels can be further increased by reduction of delta 12 fatty acid desaturase activity using the methods above, thereby decreasing the amount of oleic acid the is converted into the unsaturates linoleic acid and linolenic acid. Thus, the oil produced can have a fatty acid profile with at least 75% oleic and at most 3%, 2%, 1%, or 0.5% linoleic acid. In a related example, the oil has between 80 to 95% oleic acid and about 0.001 to 2% linoleic acid, 0.01 to 2% linoleic acid, or 0.1 to 2% linoleic acid. In another related embodiment, an oil is produced by cultivating an oleaginous cell (e.g., a microalga) so that the microbe produces a cell oil with less than 10% palmitic acid, greater than 85% oleic acid, 1% or less polyunsaturated fatty acids, and less than 7% saturated fatty acids. See, e.g., WO2015/051319, in which such an oil is produced in a microalga with FAD and FATA knockouts plus expression of an exogenous KASII gene. Such oils will have a low freezing point, with excellent stability and are useful in foods, for frying, fuels, or in chemical applications. Further, these oils may exhibit a reduced propensity to change color over time. In an illustrative chemical application, the high oleic oil is used to produce a chemical. The oleic acid double bonds of the oleic acid groups of the triglycerides in the oil can be epoxidized or hydroxylated to make a polyol. The epoxidized or hydroxylated oil can be used in a variety of applications. One such application is the production of polyurethane (including polyurethane foam) via condensation of the hydroxylated triglyceride with an isocyanate, as has been practiced with hydroxylated soybean oil or castor oil. See, e.g. US2005/0239915, US2009/0176904, US2005/0176839, US2009/0270520, and U.S. Pat. No. 4,264,743 and Zlatanic, et al, Biomacromolecules 2002, 3, 1048-1056 (2002) for examples of hydroxylation and polyurethane condensation chemistries. Suitable hydroxyl forming reactions include epoxidation of one or more double bonds of a fatty acid followed by acid catalyzed epoxide ring opening with water (to form a diol), alcohol (to form a hydroxyl ether), or an acid (to form a hydroxyl ester). There are multiple advantages of using the high-oleic/low polyunsaturated oil in producing a bio-based polyurethane: (1) the shelf-life, color or odor, of polyurethane foams may be improved; (2) the reproducibility of the product may be improved due to lack of unwanted side reactions resulting from polyunsaturates; (3) a greater degree of hydroxylation reaction may occur due to lack of polyunsaturates and the structural characteristics of the polyurethane product can be improved accordingly.

The low-polyunsaturated or high-oleic/low-polyunsaturated oils described here may be advantageously used in chemical applications where yellowing is undesirable. For example, yellowing can be undesirable in paints or coatings made from the triglycerides fatty acids derived from the triglycerides. Yellowing may be caused by reactions involving polyunsaturated fatty acids and tocotrienols and/or tocopherols. Thus, producing the high-stability oil in an oleaginous microbe with low levels of tocotrienols can be advantageous in elevating high color stability a chemical composition made using the oil. In contrast to commonly used plant oils, through appropriate choice of oleaginous microbe, the cell oils of these embodiments can have tocopherols and tocotrienols levels of 1 g/L or less. In a specific embodiment, a cell oil has a fatty acid profile with less than 2% with polyunsaturated fatty acids and less than 1 g/L for tocopherols, tocotrienols or the sum of tocopherols and tocotrienols. In another specific embodiment, the cell oil has a fatty acid profile with less than 1% with polyunsaturated fatty acids and less than 0.5 g/L for tocopherols, tocotrienols or the sum of tocopherols and tocotrienols

Any of the high-stability (low-polyunsaturate) cell oils or derivatives thereof can be used to formulate foods, drugs, vitamins, nutraceuticals, personal care or other products, and are especially useful for oxidatively sensitive products. For example, the high-stability cell oil (e.g., less than or equal to 3%, 2% or 1% polyunsaturates) can be used to formulate a sunscreen (e.g. a composition having one or more of avobenzone, homosalate, octisalate, octocrylene or oxybenzone) or retinoid face cream with an increased shelf life due to the absence of free-radical reactions associated with polyunsaturated fatty acids. For example, the shelf-life can be increased in terms of color, odor, organoleptic properties or % active compound remaining after accelerated degradation for 4 weeks at 54° C. The high stability oil can also be used as a lubricant with excellent high-temperature stability. In addition to stability, the oils can be biodegradable, which is a rare combination of properties.

In another related embodiment, the fatty acid profile of a cell oil is elevated in C8 to C16 fatty acids through additional genetic modification, e.g. through overexpression of a short-chain to mid chain preferring acyl-ACP thioesterase or other modifications described here. A low polyunsaturated oil in accordance with these embodiments can be used for various industrial, food, or consumer products, including those requiring improved oxidative stability. In food applications, the oils may be used for frying with extended life at high temperature, or extended shelf life.

Where the oil is used for frying, the high stability of the oil may allow for frying without the addition of antioxidant and/or defoamers (e.g. silicone). As a result of omitting defoamers, fried foods may absorb less oil. Where used in fuel applications, either as a triglyceride or processed into biodiesel or renewable diesel (see, e.g., WO2008/151149 WO2010/063032, and WO2011/150410), the high stability can promote storage for long periods, or allow use at elevated temperatures. For example, the fuel made from the high stability oil can be stored for use in a backup generator for more than a year or more than 5 years. The frying oil can have a smoke point of greater than 200° C., and free fatty acids of less than 0.1% (either as a cell oil or after refining).

The low polyunsaturated oils may be blended with food oils, including structuring fats such as those that form beta or beta prime crystals, including those produced as described below. These oils can also be blended with liquid oils. If mixed with an oil having linoleic acid, such as corn oil, the linoleic acid level of the blend may approximate that of high oleic plant oils such as high oleic sunflower oils (e.g., about 80% oleic and 8% linoleic).

Blends of the low polyunsaturated cell oil can be interesterified with other oils. For example, the oil can be chemically or enzymatically interesterified. In a specific embodiment, a low polyunsaturated oil according to an embodiment of the invention has at least 10% oleic acid in its fatty acid profile and less than 5% polyunsaturates and is enzymatically interesterified with a high saturate oil (e.g. hydrogenated soybean oil or other oil with high stearate levels) using an enzyme that is specific for sn-1 and sn-2 triacylglycerol positions. The result is an oil that includes a stearate-oleate-stearate (SOS). Methods for interesterification are known in the art; see for example, “Enzymes in Lipid Modification,” Uwe T. Bornschuer, ed., Wiley_VCH, 2000, ISBN 3-527-30176-3.

High stability oils can be used as spray oils. For example, dried fruits such as raisins can be sprayed with a high stability oil having less than 5, 4, 3, 2, or 1% polyunsaturates. As a result, the spray nozzle used will become clogged less frequently due to polymerization or oxidation product buildup in the nozzle that might otherwise result from the presence of polyunsaturates.

In a further embodiment, an oil that is high is SOS, such as those described below can be improved in stability by knockdown or regulation of delta 12 fatty acid desaturase.

Optionally, where the FADc promoter is regulated, it can be regulated with a promoter that is operable at low pH (e.g., one for which the level of transcription of FADc is reduced by less than half upon switching from cultivation at pH 7.0 to cultivation at pH 5.0). The promoter can be sensitive to cultivation under low nitrogen conditions such that the promoter is active under nitrogen replete conditions and inactive under nitrogen starved conditions. For example, the promoter may cause a reduction in FADc transcript levels of 5, 10, 15-fold or more upon nitrogen starvation. Because the promoter is operable at pH 5.0, more optimal invertase activity can be obtained. For example, the cell can be cultivated in the presence of invertase at a pH of less than 6.5, 6.0 or 5.5. The cell may have a FADc knockout to increase the relative gene-dosage of the regulated FADc. Optionally, the invertase is produced by the cell (natively or due to an exogenous invertase gene). Because the promoter is less active under nitrogen starved conditions, fatty acid production can proceed during the lipid production phase that would not allow for optimal cell proliferation in the cell proliferation stage. In particular, a low linoleic oil may be produced. The cell can be cultivated to an oil content of at least 20% lipid by dry cell weight. The oil may have a fatty acid profile having less than 5, 4, 3, 2, 1, or 0.5, 0.2, or 0.1% linoleic acid. WO2015/051319 describes the discovery of such promoters.

### V. Regiospecific and Stereospecific Oils/Fats

In an embodiment, a recombinant cell produces a cell fat or oil having a given regiospecific makeup. As a result, the cell can produce triglyceride fats having a tendency to form crystals of a given polymorphic form; e.g., when heated to above melting temperature and then cooled to below melting temperature of the fat. For example, the fat may tend to form crystal polymorphs of the β or β′ form (e.g., as determined by X-ray diffraction analysis), either with or without tempering. The fats may be ordered fats. In specific embodiments, the fat may directly from either β or β′ crystals upon cooling; alternatively, the fat can proceed through a β form to a β′ form. Such fats can be used as structuring, laminating or coating fats for food applications. The cell fats can be incorporated into candy, dark or white chocolate, chocolate flavored confections, ice cream, margarines or other spreads, cream fillings, pastries, or other food products. Optionally, the fats can be semi-solid (at room temperature) yet free of artificially produced trans-fatty acids. Such fats can also be useful in skin care and other consumer or industrial products.

As in the other embodiments, the fat can be produced by genetic engineering of a plastidic cell, including heterotrophic eukaryotic microalgae of the phylum Chlorophyta, the class Trebouxiophytae, the order Chlorellales, or the family Chlorellacae. Preferably, the cell is oleaginous and capable of accumulating at least 40% oil by dry cell weight. The cell can be an obligate heterotroph, such as a species of Prototheca, including Prototheca moriformis or Prototheca zopfii. The fats can also be produced in autotrophic algae or plants. Optionally, the cell is capable of using sucrose to produce oil and a recombinant invertase gene may be introduced to allow metabolism of sucrose, as described in PCT Publications WO2008/151149, WO2010/063032, WO2011/150410, WO2011/150411, and international patent application PCT/US12/23696. The invertase may be codon optimized and integrated into a chromosome of the cell, as may all of the genes mentioned here. It has been found that cultivated recombinant microalgae can produce hardstock fats at temperatures below the melting point of the hardstock fat. For example, Prototheca moriformis can be altered to heterotrophically produce triglyceride oil with greater than 50% stearic acid at temperatures in the range of 15 to 30° C., wherein the oil freezes when held at 30° C.

In an embodiment, the cell fat has at least 30, 40, 50, 60, 70, 80, or 90% fat of the general structure [saturated fatty acid (sn-1)-unsaturated fatty acid (sn-2)-saturated fatty acid (sn-3)]. This is denoted below as Sat-Unsat-Sat fat. In a specific embodiment, the saturated fatty acid in this structure is preferably stearate or palmitate and the unsaturated fatty acid is preferably oleate. As a result, the fat can form primarily β or β′ polymorphic crystals, or a mixture of these, and have corresponding physical properties, including those desirable for use in foods or personal care products. For example, the fat can melt at mouth temperature for a food product or skin temperature for a cream, lotion or other personal care product (e.g., a melting temperature of 30 to 40, or 32 to 35° C.). Optionally, the fats can have a 2 L or 3 L lamellar structure (e.g., as determined by X-ray diffraction analysis). Optionally, the fat can form this polymorphic form without tempering.

In a specific related embodiment, a cell fat triglyceride has a high concentration of SOS (i.e. triglyceride with stearate at the terminal sn-1 and sn-3 positions, with oleate at the sn-2 position of the glycerol backbone). For example, the fat can have triglycerides comprising at least 50, 60, 70, 80 or 90% SOS. In an embodiment, the fat has triglyceride of at least 80% SOS. Optionally, at least 50, 60, 70, 80 or 90% of the sn-2 linked fatty acids are unsaturated fatty acids. In a specific embodiment, at least 95% of the sn-2 linked fatty acids are unsaturated fatty acids. In addition, the SSS (tri-stearate) level can be less than 20, 10 or 5% and/or the C20:0 fatty acid (arachidic acid) level may be less than 6%, and optionally greater than 1% (e.g., from 1 to 5%). For example, in a specific embodiment, a cell fat produced by a recombinant cell has at least 70% SOS triglyceride with at least 80% sn-2 unsaturated fatty acyl moieties. In another specific embodiment, a cell fat produced by a recombinant cell has TAGs with at least 80% SOS triglyceride and with at least 95% sn-2 unsaturated fatty acyl moieties. In yet another specific embodiment, a cell fat produced by a recombinant cell has TAGs with at least 80% SOS, with at least 95% sn-2 unsaturated fatty acyl moieties, and between 1 to 6% C20 fatty acids.

In yet another specific embodiment, the sum of the percent stearate and palmitate in the fatty acid profile of the cell fat is twice the percentage of oleate, ±10, 20, 30 or 40% [e.g., (% P+% S)/% O=2.0±20%]. Optionally, the sn-2 profile of this fat is at least 40%, and preferably at least 50, 60, 70, or 80% oleate (at the sn-2 position). Also optionally, this fat may be at least 40, 50, 60, 70, 80, or 90% SOS. Optionally, the fat comprises between 1 to 6% C20 fatty acids.

In any of these embodiments, the high SatUnsatSat fat may tend to form β′ polymorphic crystals. Unlike previously available plant fats like cocoa butter, the SatUnsatSat fat produced by the cell may form β′ polymorphic crystals without tempering. In an embodiment, the polymorph forms upon heating to above melting temperature and cooling to less that the melting temperature for 3, 2, 1, or 0.5 hours. In a related embodiment, the polymorph forms upon heating to above 60° C. and cooling to 10° C. for 3, 2, 1, or 0.5 hours.

In various embodiments the fat forms polymorphs of the β form, β′ form, or both, when heated above melting temperature and the cooled to below melting temperature, and optionally proceeding to at least 50% of polymorphic equilibrium within 5, 4, 3, 2, 1, 0.5 hours or less when heated to above melting temperature and then cooled at 10° C. The fat may form β′ crystals at a rate faster than that of cocoa butter.

Optionally, any of these fats can have less than 2 mole % diacylglycerol, or less than 2 mole % mono and diacylglycerols, in sum.

In an embodiment, the fat may have a melting temperature of between 30-60° C., 30-40° C., 32 to 37° C., 40 to 60° C. or 45 to 55° C. In another embodiment, the fat can have a solid fat content (SFC) of 40 to 50%, 15 to 25%, or less than 15% at 20° C. and/or have an SFC of less than 15% at 35° C.

The cell used to make the fat may include recombinant nucleic acids operable to modify the saturate to unsaturate ratio of the fatty acids in the cell triglyceride in order to favor the formation of SatUnsatSat fat. For example, a knock-out or knock-down of stearoyl-ACP desaturase (SAD) gene can be used to favor the formation of stearate over oleate or expression of an exogenous mid-chain-preferring acyl-ACP thioesterase gene can increase the levels mid-chain saturates. Alternately a gene encoding a SAD enzyme can be overexpressed to increase unsaturates.

In a specific embodiment, the cell has recombinant nucleic acids operable to elevate the level of stearate in the cell. As a result, the concentration of SOS may be increased. WO2015/051319 demonstrates that the regiospecific profile of the recombinant microbe is enriched for the SatUnsatSat triglycerides POP, POS, and SOS as a result of overexpressing a Brassica napus C18:0-preferring thioesterase. An additional way to increase the stearate of a cell is to decrease oleate levels. For cells having high oleate levels (e.g., in excess of one half the stearate levels) one can also employ recombinant nucleic acids or classical genetic mutations operable to decrease oleate levels. For example, the cell can have a knockout, knockdown, or mutation in one or more FATA alleles, which encode an oleate liberating acyl-ACP thioesterase, and/or one or more alleles encoding a stearoyl ACP desaturase (SAD). WO2015/051319 describes the inhibition of SAD2 gene product expression using hairpin RNA to produce a fatty acid profile of 37% stearate in Prototheca moriformis (UTEX 1435), whereas the wildtype strain produced less than 4% stearate, a more than 9-fold improvement. Moreover, while such strains are engineered to reduce SAD activity, sufficient SAD activity remains to produce enough oleate to make SOS, POP, and POS. In specific examples, one of multiple SAD encoding alleles may be knocked out and/or one or more alleles are downregulated using inhibition techniques such as antisense, RNAi, or siRNA, hairpin RNA or a combination thereof. In various embodiments, the cell can produce TAGs that have 20-30, 30-40, 40-50, 50-60, 60-70, 70-80, 80-90, or 90 to about 100% stearate. In other embodiments, the cells can produce TAGs that are 20-30, 30-40, 40-50, 50-60, 60-70, 70-80, 80-90, or 90 to about 100% SOS. Optionally, or in addition to genetic modification, stearoyl ACP desaturase can be inhibited chemically; e.g., by addition of sterculic acid to the cell culture during oil production.

Surprisingly, knockout of a single FATA allele has been found to increase the presence of C18 fatty acids produced in microalgae. By knocking out one allele, or otherwise suppressing the activity of the FATA gene product (e.g., using hairpin RNA), while also suppressing the activity of stearoyl-ACP desaturase (using techniques disclosed herein), stearate levels in the cell can be increased.

Another genetic modification to increase stearate levels includes increasing a ketoacyl ACP synthase (KAS) activity in the cell so as to increase the rate of stearate production. It has been found that in microalgae, increasing KASII activity is effective in increasing C18 synthesis and particularly effective in elevating stearate levels in cell triglyceride in combination with recombinant DNA effective in decreasing SAD activity. Recombinant nucleic acids operable to increase KASII (e.g., an exogenous KasII gene) can be also be combined with a knockout or knockdown of a FatA gene, or with knockouts or knockdowns of both a FatA gene and a SAD gene). Optionally, the KASII gene encodes a protein having at least 75, 80, 85, 90, 95, 96, 97, 98, or 99% amino acid identity to the KASII Prototheca moriformis (mature protein given in SEQ ID NO: 2), or any plant KASII gene disclosed in WO2015/051319 or known in the art including a microalgal KASII.

Optionally, the cell can include an exogenous stearate-liberating acyl-ACP thioesterase, either as a sole modification or in combination with one or more other stearate-increasing genetic modifications. For example the cell may be engineered to overexpress an acyl-ACP thioesterase with preference for cleaving C18:0-ACPs. WO2015/051319 describes the expression of exogenous C18:0-preferring acyl-ACP thioesterases to increase stearate in the fatty acid profile of the microalgae, Prototheca moriformis (UTEX 1435), from about 3.7% to about 30.4% (over 8-fold). WO2015/051319 provides additional examples of C18:0-preferring acyl-ACP thioesterases function to elevate C18:0 levels in Prototheca. Optionally, the stearate-preferring acyl-ACP thioesterase gene encodes an enzyme having at least 80, 85, 90, 91, 92, 93, 94, 95, 96, 97, 98 or 9% amino acid identity to SEQ ID NOs. 3, 4, 5, 6, 7, 8, or 9, omitting FLAG tags when present. Introduction of the acyl-ACP-thioesterase can be combined with a knockout or knockdown of one or more endogenous acyl-ACP thioesterase alleles. Introduction of the thioesterase can also be combined with overexpression of an elongase (KCS) or beta-ketoacyl-CoA synthase. In addition, one or more exogenous genes (e.g., encoding SAD or KASII) can be regulated via an environmental condition (e.g., by placement in operable linkage with a regulatable promoter). In a specific example, pH and/or nitrogen level is used to regulate an amt03 promoter. The environmental condition may then be modulated to tune the cell to produce the desired amount of stearate appearing in cell triglycerides (e.g., to twice the oleate concentration). As a result of these manipulations, the cell may exhibit an increase in stearate of at least 5, 10, 15, or 20 fold.

As a further modification, alone or in combination with the other stearate increasing modifications, the cell can comprise recombinant nucleic acids operable to express an elongase or a beta-ketoacyl-CoA synthase. For example, overexpression of a C18:0-preferring acyl-ACP thioesterases may be combined with overexpression of a midchain-extending elongase or KCS to increase the production of stearate in the recombinant cell. One or more of the exogenous genes (e.g., encoding a thioesterase, elongase, or KCS) can be regulated via an environmental condition (e.g., by placement in operable linkage with a regulatable promoter). In a specific example, pH and/or nitrogen level is used to regulate an amt03 promoter or other promoters. The environmental condition may then be modulated to tune the cell to produce the desired amount of stearate appearing in cell triglycerides (e.g., to twice the oleate concentration). As a result of these manipulations, the cell may exhibit an increase in stearate of at least 5, 10, 15, or 20 fold. In addition to stearate, arachidic, behenic, lignoceric, and cerotic acids may also be produced.

In specific embodiments, due to the genetic manipulations of the cell to increase stearate levels, the ratio of stearate to oleate in the oil produced by the cell is 2:1±30% (i.e., in the range of 1.4:1 to 2.6:1), 2:1±20% or 2:1±10%.

Alternately, the cell can be engineered to favor formation of SatUnsatSat where Sat is palmitate or a mixture of palmitate and stearate. In this case introduction of an exogenous palmitate liberating acyl-ACP thioesterase can promote palmitate formation. In this embodiment, the cell can produce triglycerides, that are at least 30, 40, 50, 60, 70, or 80% POP, or triglycerides in which the sum of POP, SOS, and POS is at least 30, 40, 50, 60, 70, 80, or 90% of cell triglycerides. In other related embodiments, the POS level is at least 30, 40, 50, 60, 70, 80, or 90% of the triglycerides produced by the cell.

In a specific embodiment, the melting temperature of the oil is similar to that of cocoa butter (about 30-32° C.). The POP, POS and SOS levels can approximate cocoa butter at about 16, 38, and 23% respectively. For example, POP can be 16%±20%, POS can be 38%±20%, and SOS can be 23%±20%. Or, POP can be 16%±15%, POS can be 38%±15%, an SOS can be 23%±15%. Or, POP can be 16%±10%, POS can be 38%±10%, an SOS can be 23%±10%.

As a result of the recombinant nucleic acids that increase stearate, a proportion of the fatty acid profile may be arachidic acid. For example, the fatty acid profile can be 0.01% to 5%, 0.1 to 4%, or 1 to 3% arachidic acid. Furthermore, the regiospecific profile may have 0.01% to 4%, 0.05% to 3%, or 0.07% to 2% AOS, or may have 0.01% to 4%, 0.05% to 3%, or 0.07% to 2% AOA. It is believed that AOS and AOA may reduce blooming and fat migration in confection comprising the fats of the present invention, among other potential benefits.

In addition to the manipulations designed to increase stearate and/or palmitate, and to modify the SatUnsatSat levels, the levels of polyunsaturates may be suppressed, including as described above by reducing delta 12 fatty acid desaturase activity (e.g., as encoded by a Fad gene) and optionally supplementing the growth medium or regulating FAD expression. It has been discovered that, in microalgae (as evidenced by work in Prototheca strains), polyunsaturates are preferentially added to the sn-2 position. Thus, to elevate the percent of triglycerides with oleate at the sn-2 position, production of linoleic acid by the cell may be suppressed. The techniques described herein, in connection with highly oxidatively stable oils, for inhibiting or ablating fatty acid desaturase (FAD) genes or gene products may be applied with good effect toward producing SatUnsatSat oils by reducing polyunsaturates at the sn-2 position. As an added benefit, such oils can have improved oxidatively stability. As also described herein, the fats may be produced in two stages with polyunsaturates supplied or produced by the cell in the first stage with a deficit of polyunsaturates during the fat producing stage. The fat produced may have a fatty acid profile having less than or equal to 15, 10, 7, 5, 4, 3, 2, 1, or 0.5% polyunsaturates. In a specific embodiment, the oil/fat produced by the cell has greater than 50% SatUnsatSat, and optionally greater than 50% SOS, yet has less than 3% polyunsaturates. Optionally, polyunsaturates can be approximated by the sum of linoleic and linolenic acid area % in the fatty acid profile.

In an embodiment, the cell fat is a Shea stearin substitute having 65% to 95% SOS and optionally 0.001 to 5% SSS. In a related embodiment, the fat has 65% to 95% SOS, 0.001 to 5% SSS, and optionally 0.1 to 8% arachidic acid containing triglycerides. In another related embodiment, the fat has 65% to 95% SOS and the sum of SSS and SSO is less than 10% or less than 5%.

The cell's regiospecific preference can be learned using the analytical method described below (Examples 1-3). It is possible that the use of genetic engineering techniques, optionally combined with classical mutagenesis and breeding, a microalga or higher plant can be produced with an increase in the amount of SatUnsatSat or SOS produced of at least 1.5-fold, 2-fold, 3-fold, 4-fold, 5-fold, or more relative to the starting strain. In another aspect, the SatUnsatSat or SOS concentration of a species for which the wild-type produces less than 20%, 30%, 40% or 50% SatUnsatSat or SOS can be increased so that the SatUnsatSat or SOS is increased to at least 30%, 40%, 50% or 60%, respectively. The key changes, relative to the starting or wild-type organism, are to increase the amount of stearate (e.g., by reducing the amount of oleate formed from stearate, e.g., by reducing SAD activity, and/or increasing the amount of palmitate that is converted to stearate by reducing the activity of FATA and/or increasing the activity of KASII) and by decreasing the amount of linoleate by reducing FAD2/FADc activity.

Optionally, the starting organism can have triacylglycerol (TAG) biosynthetic machineries which are predisposed toward the synthesis of TAG species in which oleate or unsaturated fatty acids, predominate at the sn-2 position. Many oilseed crops have this characteristic. It has been demonstrated that lysophosphatidic acyltransferases (LPAATs) play a critical role in determining the species of fatty acids which will ultimately be inserted at the sn-2 position. Indeed, manipulation, through heterologous gene expression, of LPAATs in higher plant seeds, can alter the species of fatty acid occupying the sn-2 position.

One approach to generating oils with significant levels of so-called structuring fats (typically comprised of the species SOS-stearate-oleate-stearate, POS-palmitate-oleate-stearate, or POP-palmitate-oleate-palmitate) in agriculturally important oilseeds and in algae, is through the manipulation of endogenous as well as heterologous gene expression. Such approaches include:

Increasing the level of stearate. This can be achieved, as we have demonstrated in microalgae here and others have shown in higher plants, through the expression of stearate specific FATA activities or down regulation of the endogenous SAD activity; e.g., through direct gene knockout, RNA silencing, or mutation, including classical strain improvement. Simply elevating stearate levels alone, by the above approaches, however, will not be optimal. For example, in the case of palm oil, the already high levels of palmitate, coupled with increased stearate levels, will likely overwhelm the existing LPAAT activity, leading to significant amounts of stearate and palmitate incorporation into tri-saturated fatty acids (SSS, PPP, SSP, PPS etc). Hence, steps must be taken to control palmitate levels as well.

Palmitate levels must be minimized in order to create high SOS containing fats because palmitate, even with a high-functioning LPAAT, will occupy sn-1 or sn-3 positions that could be taken up by stearate, and, as outlined above, too many saturates will result in significant levels of tri-saturated TAG species. Palmitate levels can be lowered. for example, through down-regulation of endogenous FATA activity through mutation/classical strain improvement, gene knockouts or RNAi-mediated strategies, in instances wherein the endogenous FATA activity has significant palmitate activity. Alternatively, or in concert with the above, palmitate levels can be lowered through over expression of endogenous KASII activity or classical strain improvement efforts which manifest in the same effect, such that elongation from palmitate to stearate is enhanced. Simply lowering palmitate levels via the above methods may not be sufficient, however. Take again the example of palm oil. Reduction of palmitate and elevation of stearate via the previous methods would still leave significant levels of linoleic acid. The endogenous LPAAT activity in most higher plants species while they will preferentially insert oleate in the sn-2 position, will insert linoleic as the next most preferred species. As oleate levels decrease, linoleic will come to occupy the sn-2 position with increased frequency. TAG species with linoleic at the sn-2 position have poor structuring properties as the TAGs will tend to display much higher melting temperatures than what is desired in a structuring fat. Hence, increases in stearate and reductions in palmitate must in turn be balanced by reductions in levels of linoleic fatty acids.

In turn, levels of linoleic fatty acids must be minimized in order to create high SOS-containing fats because linoleate, even with a high functioning LPAAT will occupy sn-2 positions to the exclusion of oleate, creating liquid oils as opposed to the desired solid fat (at room temperature). Linoleate levels can be lowered, as we have demonstrated in microalgae and others have shown in plant oilseeds, through down regulation of endogenous FAD2 desaturases; e.g., through mutation/classical strain improvement, FAD2 knockouts or RNAi mediated down regulation of endogenous FAD2 activity. Accordingly, the linoleic acid level in the fatty acid profile can be reduced by at least 10, 20, 30, 40, 50, 100, 200, or 300%. For example, an RNAi construct with at least 70, 75, 80, 85, 90, 95, 96, 97, 98, or 99% identity to those disclosed herein can be used to downregulate FAD2.

Although one can choose a starting strain with such an sn-2 preference one can also introduce an exogenous LPAAT gene having a greater oleate preference, to further boost oleate at the sn-2 position and to further boost Sat-Unsat-Sat in the TAG profile. Optionally, one can replace one or more endogenous LPAAT alleles with the exogenous, more specific LPAAT.

The cell oils resulting from the SatUnsatSat/SOS producing organisms can be distinguished from conventional sources of SOS/POP/POS in that the sterol profile will be indicative of the host organism as distinguishable from the conventional source. Conventional sources of SOS/POP/POS include cocoa, shea, mango, sal, illipe, kokum, and allanblackia. See section XII of this disclosure for a discussion of microalgal sterols.

TABLE 6
The fatty acid profiles of some commercial oilseed strains.

| Common Food Oils* | C12:0 | C14:0 | C16:0 | C16:1 | C18:0 | C18:1 | C18:2 | C18:3 |
|---|---|---|---|---|---|---|---|---|
| Corn oil (Zea mays) | | <1.0 | 8.0-19.0 | <0.5 | 0.5-4.0 | 19-50 | 38-65 | <2.0 |
| Cottonseed oil (Gossypium barbadense) | <0.1 | 0.5-2.0 | 17-29 | <1.5 | 1.0-4.0 | 13-44 | 40-63 | 0.1-2.1 |
| Canola (Brassica rapa, B. napus, B. juncea) | <0.1 | <0.2 | <6.0 | <1.0 | <2.5 | >50 | <40 | <14 |
| Olive (Olea europea) | | <0.1 | 6.5-20.0 | ≤3.5 | 0.5-5.0 | 56-85 | 3.5-20.0 | ≤1.2 |
| Peanut (Arachis hypogaea) | <0.1 | <0.2 | 7.0-16.0 | <1.0 | 1.3-6.5 | 35-72 | 13.0-43 | <0.6 |
| Palm (Elaeis guineensis) | | 0.5-5.9 | 32.0-47.0 | | 2.0-8.0 | 34-44 | 7.2-12.0 | |
| Safflower (Carthamus tinctorus) | <0.1 | <1.0 | 2.0-10.0 | <0.5 | 1.0-10.0 | 7.0-16.0 | 72-81 | <1.5 |
| Sunflower (Helianthus annus) | <0.1 | <0.5 | 3.0-10.0 | <1.0 | 1.0-10.0 | 14-65 | 20-75 | <0.5 |
| Soybean (Glycine max) | <0.1 | <0.5 | 7.0-12.0 | <0.5 | 2.0-5.5 | 19-30 | 48-65 | 5.0-10.0 |
| Solin-Flax (Linum usitatissimum) | <0.1 | <0.5 | 2.0-9.0 | <0.5 | 2.0-5.0 | 8.0-60 | 40-80 | <5.0 |

*Unless otherwise indicated, data taken from the U.S. Pharacopeia's Food and Chemicals Codex, 7th Ed. 2010-2011**

Accordingly, in an embodiment of the present invention, there is a method for increasing the amount of SOS in an oil (i.e. oil or fat) produced by a cell. The method comprises providing a cell and using classical and/or genetic engineering techniques (e.g., mutation, selection, strain-improvement, introduction of an exogenous gene and/or regulator element, or RNA-level modulation such as RNAi) to (i) increase the stearate in the oil, (ii) decrease the linoleate in the oil, and optionally (iii) increase the stereospecificity of the addition of oleate in the sn-2 position. The step of increasing the stearate can comprise decreasing desaturation by SAD (e.g., knockout, knockdown or use of regulatory elements) and increasing the conversion of palmitate to stearate (including overexpression of an endogenous or exogenous KASII and/or knockout or knockdown of FATA). Optionally, an exogenous FATA with greater stearate specificity then an endogenous FATA is expressed in the cell to increase stearate levels. Here, stearate-specificity of a FATA gene is a measure of the gene product's rate of cleavage of stearate over palmitate. The stearate-specific FATA gene insertion can be combined with a knockdown or knockout of the less-specific endogenous FATA gene. In this way, the ratio of stearate to palmitate can be increased, by 10%, 20%, 30%, 40%, 50%, 100% or more. The step of decreasing the linoleate can be via reduction of FADc/FAD2 activity including knockout and/or knockdown. The step of increasing the oleate at the sn-2 position can comprise expressing an exogenous oleate-preferring LPAAT such as an LPAAT having at least 75, 80, 85, 90, 85, 96, 97, 98, or 99% amino acid identity to an LPAAT disclosed herein.

In a specific embodiment, the cell (e.g., an oleaginous microalgal or other plastidic cell) produces an oil enriched in SOS (e.g., at least 50% SOS and in some cases 60% SOS). The cell is modified in at least four genes: (i) a β-ketoacyl-ACP synthase II (KASII) is overexpressed, (ii) activity of an endogenous FATA acyl-ACP thioesterase is reduced (iii) a stearate-specific FATA acyl-ACP thioesterase is overexpressed, (iii) endogenous SAD activity is decreased, and (iv) endogenous FAD activity is decreased. WO2015/051319 demonstrates this embodiment in a Prototheca moriformis microalga by disrupting the coding region of endogenous FATA and SAD2 through homologous recombination, overexpressing a β-ketoacyl-ACP synthase II (KASII) gene, and activating FAD2 RNAi to decrease polyunsaturates.

In another specific embodiment, the cell (e.g., an oleaginous microalgal or other plastidic cell) produces an oil enriched in SOS (e.g., at least 50% SOS and in some cases 60% SOS). The cell is modified in at least four genes: (i) a β-ketoacyl-ACP synthase II (KASII) is overexpressed, (ii) activity of an endogenous FATA acyl-ACP thioesterase is reduced (iii) a stearate-specific FATA acyl-ACP thioesterase is overexpressed, (iv) endogenous SAD activity is decreased, (v) endogenous FAD activity is decreased and (vi) an exogenous oleate-preferring LPAAT is expressed. Optionally, these genes or regulatory elements have at least 75, 80, 85, 90, 85, 96, 97, 98, or 99% nucleic acid or amino acid identity to a gene or gene-product or regulatory element disclosed herein. Optionally, one or more of these genes is under control of a pH-sensitive or nitrogen-sensitive (pH-sensitive or pH-insensitive) promoter such as one having at least 75, 80, 85, 90, 85, 96, 97, 98, or 99% nucleic acid identity to one of those disclosed herein. Optionally, the cell oil is fractionated.

In an embodiment, fats produced by cells according to the invention are used to produce a confection, candy coating, or other food product. As a result, a food product like a chocolate or candy bar may have the “snap” (e.g., when broken) of a similar product produced using cocoa butter. The fat used may be in a beta polymorphic form or tend to a beta polymorphic form. In an embodiment, a method includes adding such a fat to a confection. Optionally, the fat can be a cocoa butter equivalent per EEC regulations, having greater than 65% SOS, less than 45% unsaturated fatty acid, less than 5% polyunsaturated fatty acids, less than 1% lauric acid, and less than 2% trans fatty acid. The fats can also be used as cocoa butter extenders, improvers, replacers, or anti-blooming agents, or as Shea butter replacers, including in food and personal care products. High SOS fats produced using the cells and methods disclosed here can be used in any application or formulation that calls for Shea butter or Shea fraction. However, unlike Shea butter, fats produced by the embodiments of the invention can have low amounts of unsaponifiables; e.g. less than 7, 5, 3, or 2% unsaponifiables. In addition, Shea butter tends to degrade quickly due to the presence of diacylglycerides whereas fats produced by the embodiments of the invention can have low amounts of diacylglycerides; e.g., less than 5, 4, 3, 2, 1, or 0.5% diacylglycerides.

In an embodiment of the invention there is a cell fat suitable as a shortening, and in particular, as a roll-in shortening. Thus, the shortening may be used to make pastries or other multi-laminate foods. The shortening can be produced using methods disclosed herein for producing engineered organisms and especially heterotrophic microalgae. In an embodiment, the shortening has a melting temperature of between 40 to 60° C. and preferably between 45-55° C. and can have a triglyceride profile with 15 to 20% medium chain fatty acids (C8 to C14), 45-50% long chain saturated fatty acids (C16 and higher), and 30-35% unsaturated fatty acids (preferably with more oleic than linoleic). The shortening may form β′ polymorphic crystals, optionally without passing through the β polymorphic form. The shortening may be thixotropic. The shortening may have a solid fat content of less than 15% at 35° C. In a specific embodiment, there is a cell oil suitable as a roll-in shortening produced by a recombinant microalga, where the oil has a yield stress between 400 and 700 or 500 and 600 Pa and a storage modulus of greater than 1×10⁵ Pa or 1×10⁶ Pa (see Example 4).

A structured solid-liquid fat system can be produced using the structuring oils by blending them with an oil that is a liquid at room temperature (e.g., an oil high in tristearin or triolein). The blended system may be suitable for use in a food spread, mayonnaise, dressing, shortening; i.e. by forming an oil-water-oil emulsion. The structuring fats according to the embodiments described here, and especially those high in SOS, can be blended with other oils/fats to make a cocoa butter equivalent, replacer, or extender. For example, a cell fat having greater than 65% SOS can be blended with palm mid-fraction to make a cocoa butter equivalent.

In general, such high Sat-Unsat-Sat fats or fat systems can be used in a variety of other products including whipped toppings, margarines, spreads, salad dressings, baked goods (e.g. breads, cookies, crackers muffins, and pastries), cheeses, cream cheese, mayonnaise, etc.

In a specific embodiment, a Sat-Unsat-Sat fat described above is used to produce a margarine, spread, or the like. For example, a margarine can be made from the fat using any of the recipes or methods found in U.S. Pat. Nos. 7,118,773, 6,171,636, 4,447,462, 5,690,985, 5,888,575, 5,972,412, 6,171,636, or international patent publications WO9108677A1.

In an embodiment, a fat comprises a cell (e.g., from microalgal cells) fat optionally blended with another fat and is useful for producing a spread or margarine or other food product is produced by the genetically engineered cell and has glycerides derived from fatty acids which comprises:
(a) at least 10 weight % of C18 to C24 saturated fatty acids,
(b) which comprise stearic and/or arachidic and/or behenic and/or lignoceric acid and
(c) oleic and/or linoleic acid, while
(d) the ratio of saturated C18 acid/saturated (C20+C22+C24)-acids ≥1, preferably ≥5, more preferably ≥10, which glycerides contain:
(e) ≤5 weight % of linolenic acid calculated on total fatty acid weight
(f) ≤5 weight % of trans fatty acids calculated on total fatty acid weight
(g) ≤75 weight %, preferably ≤60 weight % of oleic acid at the sn-2 position: which glycerides contain calculated on total glycerides weight
(h) ≥8 weight % HOH+HHO triglycerides
(i) ≤5 weight % of trisaturated triglycerides, and optionally one or more of the following properties:
(j) a solid fat content of >10% at 10° C.
(k) a solid fat content ≤15% at 35° C.,
(l) a solid fat content of >15% at 10° C. and a solid fat content ≤25% at 35° C.,
(m) the ratio of (HOH+HHO) and (HLH+HHL) triglycerides is >1, and preferably >2,
   where H stands for C18-C24 saturated fatty acid, O for oleic acid, and L for linoleic acid.

Optionally, the solid content of the fat (% SFC) is 11 to 30 at 10° C., 4 to 15 at 20° C., 0.5 to 8 at 30° C., and 0 to 4 at 35° C. Alternately, the % SFC of the fat is 20 to 45 at 10° C., 14 to 25 at 20° C., 2 to 12 at 30° C., and 0 to 5 at 35° C. In related embodiment, the % SFC of the fat is 30 to 60 at 10° C., 20 to 55 at 20° C., 5 to 35 at 30° C., and 0 to 15 at 35° C. The C12-C16 fatty acid content can be ≤15 weight %. The fat can have ≤5 weight % disaturated diglycerides.

In related embodiments there is a spread, margarine or other food product made with the cell oil or cell oil blend. For example, the cell fat can be used to make an edible W/O (water/oil) emulsion spread comprising 70-20 wt. % of an aqueous phase dispersed in 30-80 wt. % of a fat phase which fat phase is a mixture of 50-99 wt. % of a vegetable triglyceride oil A and 1-50 wt. % of a structuring triglyceride fat B, which fat consists of 5-100 wt. % of a hardstock fat C and up to 95 wt. % of a fat D, where at least 45 wt. % of the hardstock fat C triglycerides consist of SatOSat triglycerides and where Sat denotes a fatty acid residue with a saturated C18-C24 carbon chain and O denotes an oleic acid residue and with the proviso that any hardstock fat C which has been obtained by fractionation, hydrogenation, esterification or interesterification of the fat is excluded. The hardstock fat can be a cell fat produced by a cell according to the methods disclosed herein. Accordingly, the hardstock fat can be a fat having a regiospecific profile having at least 50, 60, 70, 80, or 90% SOS. The W/O emulsion can be prepared to methods known in the art including in U.S. Pat. No. 7,118,773.

In related embodiment, the cell also expresses an endogenous hydrolyase enzyme that produces ricinoleic acid. As a result, the oil (e.g., a liquid oil or structured fat) produced may be more easily emulsified into a margarine, spread, or other food product or non-food product. For example, the oil produced may be emulsified using no added emulsifiers or using lower amounts of such emulsifiers. The U.S. patent application Ser. No. 13/365,253 discloses methods for expressing such hydroxylases in microalgae and other cells. In specific embodiments, a cell oil comprises at least 1, 2, or 5% SRS, where S is stearate and R is ricinoleic acid.

In an alternate embodiment, a cell oil that is a cocoa butter mimetic as described above (or other high sat-unsat-sat oil such as a Shea or Kolum mimetic) can be fractionated to remove trisaturates (e.g., tristearin and tripalmitin, SSP, and PPS). For example, it has been found that microalgae engineered to decrease SAD activity to increase SOS concentration make an oil that can be fractionated to remove trisaturated. In specific embodiments, the melting temperature of the fractionated cell oil is similar to that of cocoa butter (about 30-32° C.). The POP, POS and SOS levels can approximate cocoa butter at about 16, 38, and 23% respectively. For example, POP can be 16%±20%, POS can be 38%±20%, an SOS can be 23%±20%. Or, POP can be 16%±15%, POS can be 38%±15%, an SOS can be 23%±15%. Or, POP can be 16%±10%, POS can be 38%±10%, an SOS can be 23%±10%. In addition, the tristearin levels can be less than 5% of the triacylglycerides.

In an embodiment, a method comprises obtaining a cell oil obtained from a genetically engineered (e.g., microalga or other microbe) cell that produces a starting oil with a TAG profile having at least 40, 50, or 60% SOS. Optionally, the cell comprises one or more of an overexpressed KASII gene, a SAD knockout or knockdown, or an exogenous C18-preferring FATA gene, an exogenous LPAAT, and a FAD2 knockout or knockdown. The oil is fractionated by dry fractionation or solvent fractionation to give an enriched oil (stearin fraction) that is increased in SOS and decreased in trisaturates relative to the starting oil. The enriched oil can have at least 60%, 70% or 80% SOS with no more than 5%, 4%, 3%, 2% or 1% trisaturates. The enriched oil can have a sn-2 profile having 85, 90, 95% or more oleate at the sn-2 position. For example, the fractionated oil can comprise at least 60% SOS, no more than 5% trisaturates and at least 85% oleate at the sn-2 position. Alternatively, the oil can comprise at least 70% SOS, no more than 4% trisaturates and at least 90% oleate at the sn-2 position or 80% SOS, no more than 4% trisaturates and at least 95% oleate at the sn-2 position. Optionally, the oil has essentially identical maximum heat-flow temperatures and/or the DSC-derived SFC curves to Kokum butter. The stearin fraction can be obtained by dry fractionation, solvent fractionation, or a combination of these. Optionally, the process includes a 2-step dry fractionation at a first temperature and a second temperature. The first temperature can be higher or lower than the second temperature. In a specific embodiment, the first temperature is effective at removing OOS and the second temperature is effective in removing trisaturates. Optionally, the stearin fraction is washed with a solvent (e.g. acetone) to remove the OOS after treatment at the first temperature. Optionally, the first temperature is about 24° C. and the second temperature is about 29° C.

### VI. High Mid-Chain Oils

In an embodiment of the present invention, the cell has recombinant nucleic acids operable to elevate the level of midchain fatty acids (e.g., C8:0, C10:0, C12:0, C14:0, or C16:0 fatty acids) in the cell or in the oil of the cell. One way to increase the levels of midchain fatty acids in the cell or in the oil of the cell is to engineer a cell to express an exogenous acyl-ACP thioesterase that has activity towards midchain fatty acyl-ACP substrates (e.g., one encoded by a FatB gene), either as a sole modification or in combination with one or more other genetic modifications. Examples of such engineering can be found in, for example, WO 2015/051319.

Alternately, or in addition, the cell may comprise recombinant nucleic acids that are operable to express an exogenous KASI or KASIV enzyme and optionally to decrease or eliminate the activity of a KASII, which is particularly advantageous when a mid-chain-preferring acyl-ACP thioesterase is expressed. WO2015/051319 describes the engineering of Prototheca cells to overexpress KASI or KASIV enzymes in conjunction with a mid-chain preferring acyl-ACP thioesterase to generate strains in which production of C10-C12 fatty acids is about 59% of total fatty acids. Mid-chain production can also be increased by suppressing the activity of KASI and/or KASII (e.g., using a knockout or knockdown). WO2015/051319 details the chromosomal knockout of different alleles of Prototheca moriformis (UTEX 1435) KASI in conjunction with overexpression of a mid-chain preferring acyl-ACP thioesterase to achieve fatty acid profiles that are about 76% or 84% C10-C14 fatty acids. WO2015/051319 also provides recombinant cells and oils characterized by elevated midchain fatty acids as a result of expression of KASI RNA hairpin polynucleotides. In addition to any of these modifications, unsaturated or polyunsaturated fatty acid production can be suppressed (e.g., by knockout or knockdown) of a SAD or FAD enzyme.

### VII. High Oleic/Palmitic Oil

In another embodiment, there is a high oleic oil with about 60% oleic acid, 25% palmitic acid and optionally 5% polyunsaturates or less. The high oleic oil can be produced using the methods disclosed in U.S. patent application Ser. No. 13/365,253, which is incorporated by reference in relevant part. For example, the cell can have nucleic acids operable to suppress an acyl-ACP thioesterase (e.g., knockout or knockdown of a gene encoding FATA) while also expressing a gene that increases KASII activity. The cell can have further modifications to inhibit expression of delta 12 fatty acid desaturase, including regulation of gene expression as described above. As a result, the polyunsaturates can be less than or equal to 5, 4, 3, 2, or 1 area %.

### VIII. Low Saturate Oil

In an embodiment, a cell oil is produced from a recombinant cell. The oil produced has a fatty acid profile that has less that 4%, 3%, 2%, or 1% (area %), saturated fatty acids. In a specific embodiment, the oil has 0.1 to 3.5% saturated fatty acids. Certain of such oils can be used to produce a food with negligible amounts of saturated fatty acids. Optionally, these oils can have fatty acid profiles comprising at least 90% oleic acid or at least 90% oleic acid with at least 3% polyunsaturated fatty acids. In an embodiment, a cell oil produced by a recombinant cell comprises at least 90% oleic acid, at least 3% of the sum of linoleic and linolenic acid and has less than 3.5% saturated fatty acids. In a related embodiment, a cell oil produced by a recombinant cell comprises at least 90% oleic acid, at least 3% of the sum of linoleic and linolenic acid and has less than 3.5% saturated fatty acids, the majority of the saturated fatty acids being comprised of chain length 10 to 16. These oils may be produced by recombinant oleaginous cells including but not limited to those described here and in U.S. patent application Ser. No. 13/365,253. For example, overexpression of a KASII enzyme in a cell with a highly active SAD can produce a high oleic oil with less than or equal to 3.5% saturates. Optionally, an oleate-specific acyl-ACP thioesterase is also overexpressed and/or an endogenous thioesterase having a propensity to hydrolyze acyl chains of less than C18 knocked out or suppressed. The oleate-specific acyl-ACP thioesterase may be a transgene with low activity toward ACP-palmitate and ACP-stearate so that the ratio of oleic acid relative to the sum of palmitic acid and stearic acid in the fatty acid profile of the oil produced is greater than 3, 5, 7, or 10. Alternately, or in addition, a FATA gene may be knocked out or knocked down, as in WO 2015/051319. A FATA gene may be knocked out or knocked down and an exogenous KASII overexpressed. Another optional modification is to increase KASI and/or KASIII activity, which can further suppress the formation of shorter chain saturates. Optionally, one or more acyltransferases (e.g., an LPAAT) having specificity for transferring unsaturated fatty acyl moieties to a substituted glycerol is also overexpressed and/or an endogenous acyltransferase is knocked out or attenuated. An additional optional modification is to increase the activity of KCS enzymes having specificity for elongating unsaturated fatty acids and/or an endogenous KCS having specificity for elongating saturated fatty acids is knocked out or attenuated. Optionally, oleate is increased at the expense of linoleate production by knockout or knockdown of a delta 12 fatty acid desaturase; e.g., using the techniques of Section IV of this patent application. Optionally, the exogenous genes used can be plant genes; e.g., obtained from cDNA derived from mRNA found in oil seeds.

### IX. Minor Oil Components

The oils produced according to the above methods in some cases are made using a microalgal host cell. As described above, the microalga can be, without limitation, fall in the classification of Chlorophyta, Trebouxiophyceae, Chlorellales, Chlorellaceae, or Chlorophyceae. It has been found that microalgae of Trebouxiophyceae can be distinguished from vegetable oils based on their sterol profiles. Oil produced by Chlorella protothecoides was found to produce sterols that appeared to be brassicasterol, ergosterol, campesterol, stigmasterol, and β-sitosterol, when detected by GC-MS. However, it is believed that all sterols produced by Chlorella have C2413 stereochemistry. Thus, it is believed that the molecules detected as campesterol, stigmasterol, and β-sitosterol, are actually 22,23-dihydrobrassicasterol, poriferasterol and clionasterol, respectively. Thus, the oils produced by the microalgae described above can be distinguished from plant oils by the presence of sterols with C2413 stereochemistry and the absence of C24a stereochemistry in the sterols present. For example, the oils produced may contain 22, 23-dihydrobrassicasterol while lacking campesterol; contain clionasterol, while lacking in β-sitosterol, and/or contain poriferasterol while lacking stigmasterol. Alternately, or in addition, the oils may contain significant amounts of Δ⁷-poriferasterol.

In one embodiment, the oils provided herein are not vegetable oils. Vegetable oils are oils extracted from plants and plant seeds. Vegetable oils can be distinguished from the non-plant oils provided herein on the basis of their oil content. A variety of methods for analyzing the oil content can be employed to determine the source of the oil or whether adulteration of an oil provided herein with an oil of a different (e.g. plant) origin has occurred. The determination can be made on the basis of one or a combination of the analytical methods. These tests include but are not limited to analysis of one or more of free fatty acids, fatty acid profile, total triacylglycerol content, diacylglycerol content, peroxide values, spectroscopic properties (e.g. UV absorption), sterol profile, sterol degradation products, antioxidants (e.g. tocopherols), pigments (e.g. chlorophyll), d13C values and sensory analysis (e.g. taste, odor, and mouth feel). Many such tests have been standardized for commercial oils such as the Codex Alimentarius standards for edible fats and oils.

Sterol profile analysis is a particularly well-known method for determining the biological source of organic matter. Campesterol, b-sitosterol, and stigmasterol are common plant sterols, with b-sitosterol being a principle plant sterol. For example, b-sitosterol was found to be in greatest abundance in an analysis of certain seed oils, approximately 64% in corn, 29% in rapeseed, 64% in sunflower, 74% in cottonseed, 26% in soybean, and 79% in olive oil (Gul et al. J. Cell and Molecular Biology 5:71-79, 2006).

Oil isolated from Prototheca moriformis strain UTEX1435 were separately clarified (CL), refined and bleached (RB), or refined, bleached and deodorized (RBD) and were tested for sterol content according to the procedure described in JAOCS vol. 60, no. 8, August 1983. Results of the analysis are shown below (units in mg/100 g) in Table 7.

TABLE 7
Sterol profiles of oils from UTEX 1435.

| | | | | | Refined, |
|---|---|---|---|---|---|
| | | | | Refined | bleached, |
| | | | | & | & |
| | Sterol | Crude | Clarified | bleached | deodorized |
| 1 | Ergosterol | 384 | 398 | 293 | 302 |
| | | (56%) | (55%) | (50%) | (50%) |
| 2 | 5,22-cholestadien- | 14.6 | 18.8 | 14 | 15.2 |
| | 24-methyl-3-ol | (2.1%) | (2.6%) | (2.4%) | (2.5%) |
| | (Brassicasterol) | | | | |
| 3 | 24-methylcholest- | 10.7 | 11.9 | 10.9 | 10.8 |
| | 5-en-3-ol | (1.6%) | (1.6%) | (1.8%) | (1.8%) |
| | (Campesterol or | | | | |
| | 22,23- | | | | |
| | dihydrobrassicasterol) | | | | |
| 4 | 5,22-cholestadien- | 57.7 | 59.2 | 46.8 | 49.9 |
| | 24-ethyl-3-ol | (8.4%) | (8.2%) | (7.9%) | (8.3%) |
| | (Stigmasterol or | | | | |
| | poriferasterol) | | | | |
| 5 | 24-ethylcholest- | 9.64 | 9.92 | 9.26 | 10.2 |
| | 5-en-3-ol | (1.4%) | (1.4%) | (1.6%) | (1.7%) |
| | (β-Sitosterol or | | | | |
| | clionasterol) | | | | |
| 6 | Other sterols | 209 | 221 | 216 | 213 |
| | Total sterols | 685.64 | 718.82 | 589.96 | 601.1 |

These results show three striking features. First, ergosterol was found to be the most abundant of all the sterols, accounting for about 50% or more of the total sterols. The amount of ergosterol is greater than that of campesterol, β-sitosterol, and stigmasterol combined. Ergosterol is steroid commonly found in fungus and not commonly found in plants, and its presence particularly in significant amounts serves as a useful marker for non-plant oils. Secondly, the oil was found to contain brassicasterol. With the exception of rapeseed oil, brassicasterol is not commonly found in plant based oils. Thirdly, less than 2% β-sitosterol was found to be present. β-sitosterol is a prominent plant sterol not commonly found in microalgae, and its presence particularly in significant amounts serves as a useful marker for oils of plant origin. In summary, Prototheca moriformis strain UTEX1435 has been found to contain both significant amounts of ergosterol and only trace amounts of β-sitosterol as a percentage of total sterol content. Accordingly, the ratio of ergosterol:β-sitosterol or in combination with the presence of brassicasterol can be used to distinguish this oil from plant oils.

In some embodiments, the oil content of an oil provided herein contains, as a percentage of total sterols, less than 20%, 15%, 10%, 5%, 4%, 3%, 2%, or 1% β-sitosterol. In other embodiments the oil is free from β-sitosterol. For any of the oils or cell-oils disclosed in this application, the oil can have the sterol profile of any column of Table 7, above, with a sterol-by-sterol variation of 30%, 20%, 10% or less.

In some embodiments, the oil is free from one or more of β-sitosterol, campesterol, or stigmasterol. In some embodiments the oil is free from β-sitosterol, campesterol, and stigmasterol. In some embodiments the oil is free from campesterol. In some embodiments the oil is free from stigmasterol.

In some embodiments, the oil content of an oil provided herein comprises, as a percentage of total sterols, less than 20%, 15%, 10%, 5%, 4%, 3%, 2%, or 1% 24-ethylcholest-5-en-3-ol. In some embodiments, the 24-ethylcholest-5-en-3-ol is clionasterol. In some embodiments, the oil content of an oil provided herein comprises, as a percentage of total sterols, at least 1%, 2%, 3%, 4%, 5%, 6%, 7%, 8%, 9%, or 10% clionasterol.

In some embodiments, the oil content of an oil provided herein contains, as a percentage of total sterols, less than 20%, 15%, 10%, 5%, 4%, 3%, 2%, or 1% 24-methylcholest-5-en-3-ol. In some embodiments, the 24-methylcholest-5-en-3-ol is 22, 23-dihydrobrassicasterol. In some embodiments, the oil content of an oil provided herein comprises, as a percentage of total sterols, at least 1%, 2%, 3%, 4%, 5%, 6%, 7%, 8%, 9%, or 10% 22,23-dihydrobrassicasterol.

In some embodiments, the oil content of an oil provided herein contains, as a percentage of total sterols, less than 20%, 15%, 10%, 5%, 4%, 3%, 2%, or 1% 5,22-cholestadien-24-ethyl-3-ol. In some embodiments, the 5, 22-cholestadien-24-ethyl-3-ol is poriferasterol. In some embodiments, the oil content of an oil provided herein comprises, as a percentage of total sterols, at least 1%, 2%, 3%, 4%, 5%, 6%, 7%, 8%, 9%, or 10% poriferasterol.

In some embodiments, the oil content of an oil provided herein contains ergosterol or brassicasterol or a combination of the two. In some embodiments, the oil content contains, as a percentage of total sterols, at least 5%, 10%, 20%, 25%, 35%, 40%, 45%, 50%, 55%, 60%, or 65% ergosterol. In some embodiments, the oil content contains, as a percentage of total sterols, at least 25% ergosterol. In some embodiments, the oil content contains, as a percentage of total sterols, at least 40% ergosterol. In some embodiments, the oil content contains, as a percentage of total sterols, at least 5%, 10%, 20%, 25%, 35%, 40%, 45%, 50%, 55%, 60%, or 65% of a combination of ergosterol and brassicasterol.

In some embodiments, the oil content contains, as a percentage of total sterols, at least 1%, 2%, 3%, 4% or 5% brassicasterol. In some embodiments, the oil content contains, as a percentage of total sterols less than 10%, 9%, 8%, 7%, 6%, or 5% brassicasterol.

In some embodiments the ratio of ergosterol to brassicasterol is at least 5:1, 10:1, 15:1, or 20:1.

In some embodiments, the oil content contains, as a percentage of total sterols, at least 5%, 10%, 20%, 25%, 35%, 40%, 45%, 50%, 55%, 60%, or 65% ergosterol and less than 20%, 15%, 10%, 5%, 4%, 3%, 2%, or 1% β-sitosterol. In some embodiments, the oil content contains, as a percentage of total sterols, at least 25% ergosterol and less than 5% β-sitosterol. In some embodiments, the oil content further comprises brassicasterol.

Sterols contain from 27 to 29 carbon atoms (C27 to C29) and are found in all eukaryotes. Animals exclusively make C27 sterols as they lack the ability to further modify the C27 sterols to produce C28 and C29 sterols. Plants however are able to synthesize C28 and C29 sterols, and C28/C29 plant sterols are often referred to as phytosterols. The sterol profile of a given plant is high in C29 sterols, and the primary sterols in plants are typically the C29 sterols b-sitosterol and stigmasterol. In contrast, the sterol profile of non-plant organisms contain greater percentages of C27 and C28 sterols. For example the sterols in fungi and in many microalgae are principally C28 sterols. The sterol profile and particularly the striking predominance of C29 sterols over C28 sterols in plants has been exploited for determining the proportion of plant and marine matter in soil samples (Huang, Wen-Yen, Meinschein W. G., “Sterols as ecological indicators”; Geochimica et Cosmochimia Acta. Vol 43. pp 739-745).

In some embodiments the primary sterols in the microalgal oils provided herein are sterols other than b-sitosterol and stigmasterol. In some embodiments of the microalgal oils, C29 sterols make up less than 50%, 40%, 30%, 20%, 10%, or 5% by weight of the total sterol content.

In some embodiments the microalgal oils provided herein contain C28 sterols in excess of C29 sterols. In some embodiments of the microalgal oils, C28 sterols make up greater than 50%, 60%, 70%, 80%, 90%, or 95% by weight of the total sterol content. In some embodiments the C28 sterol is ergosterol. In some embodiments the C28 sterol is brassicasterol.

### X. Chemical Modification

The oils of the present invention can be chemically modified. One such chemical modification is hydrogenation, which is the addition of hydrogen to double bonds in the fatty acid constituents of glycerolipids or of free fatty acids. The hydrogenation process permits the transformation of liquid oils into semi-solid or solid fats, which may be more suitable for specific applications.

Hydrogenation of oil produced by the methods described herein can be performed in conjunction with one or more of the methods and/or materials provided herein, as reported in the following: U.S. Pat. No. 7,288,278 (Food additives or medicaments); U.S. Pat. No. 5,346,724 (Lubrication products); U.S. Pat. No. 5,475,160 (Fatty alcohols); U.S. Pat. No. 5,091,116 (Edible oils); U.S. Pat. No. 6,808,737 (Structural fats for margarine and spreads); U.S. Pat. No. 5,298,637 (Reduced-calorie fat substitutes); U.S. Pat. No. 6,391,815 (Hydrogenation catalyst and sulfur adsorbent); U.S. Pat. Nos. 5,233,099 and 5,233,100 (Fatty alcohols); U.S. Pat. No. 4,584,139 (Hydrogenation catalysts); U.S. Pat. No. 6,057,375 (Foam suppressing agents); and U.S. Pat. No. 7,118,773 (Edible emulsion spreads).

One skilled in the art will recognize that various processes may be used to hydrogenate carbohydrates. One suitable method includes contacting the carbohydrate with hydrogen or hydrogen mixed with a suitable gas and a catalyst under conditions sufficient in a hydrogenation reactor to form a hydrogenated product. The hydrogenation catalyst generally can include Cu, Re, Ni, Fe, Co, Ru, Pd, Rh, Pt, Os, Ir, and alloys or any combination thereof, either alone or with promoters such as W, Mo, Au, Ag, Cr, Zn, Mn, Sn, B, P, Bi, and alloys or any combination thereof. Other effective hydrogenation catalyst materials include either supported nickel or ruthenium modified with rhenium. In an embodiment, the hydrogenation catalyst also includes any one of the supports, depending on the desired functionality of the catalyst. The hydrogenation catalysts may be prepared by methods known to those of ordinary skill in the art.

In some embodiments the hydrogenation catalyst includes a supported Group VIII metal catalyst and a metal sponge material (e.g., a sponge nickel catalyst). Raney nickel provides an example of an activated sponge nickel catalyst suitable for use in this invention. In other embodiment, the hydrogenation reaction in the invention is performed using a catalyst comprising a nickel-rhenium catalyst or a tungsten-modified nickel catalyst. One example of a suitable catalyst for the hydrogenation reaction of the invention is a carbon-supported nickel-rhenium catalyst.

In an embodiment, a suitable Raney nickel catalyst may be prepared by treating an alloy of approximately equal amounts by weight of nickel and aluminum with an aqueous alkali solution, e.g., containing about 25 weight % of sodium hydroxide. The aluminum is selectively dissolved by the aqueous alkali solution resulting in a sponge shaped material comprising mostly nickel with minor amounts of aluminum. The initial alloy includes promoter metals (i.e., molybdenum or chromium) in the amount such that about 1 to 2 weight % remains in the formed sponge nickel catalyst. In another embodiment, the hydrogenation catalyst is prepared using a solution of ruthenium (III) nitrosylnitrate, ruthenium (III) chloride in water to impregnate a suitable support material. The solution is then dried to form a solid having a water content of less than about 1% by weight. The solid may then be reduced at atmospheric pressure in a hydrogen stream at 300° C. (uncalcined) or 400° C. (calcined) in a rotary ball furnace for 4 hours. After cooling and rendering the catalyst inert with nitrogen, 5% by volume of oxygen in nitrogen is passed over the catalyst for 2 hours.

In certain embodiments, the catalyst described includes a catalyst support. The catalyst support stabilizes and supports the catalyst. The type of catalyst support used depends on the chosen catalyst and the reaction conditions. Suitable supports for the invention include, but are not limited to, carbon, silica, silica-alumina, zirconia, titania, ceria, vanadia, nitride, boron nitride, heteropolyacids, hydroxyapatite, zinc oxide, chromia, zeolites, carbon nanotubes, carbon fullerene and any combination thereof.

The catalysts used in this invention can be prepared using conventional methods known to those in the art. Suitable methods may include, but are not limited to, incipient wetting, evaporative impregnation, chemical vapor deposition, wash-coating, magnetron sputtering techniques, and the like.

The conditions for which to carry out the hydrogenation reaction will vary based on the type of starting material and the desired products. One of ordinary skill in the art, with the benefit of this disclosure, will recognize the appropriate reaction conditions. In general, the hydrogenation reaction is conducted at temperatures of 80° C. to 250° C., and preferably at 90° C. to 200° C., and most preferably at 100° C. to 150° C. In some embodiments, the hydrogenation reaction is conducted at pressures from 500 KPa to 14000 KPa.

The hydrogen used in the hydrogenolysis reaction of the current invention may include external hydrogen, recycled hydrogen, in situ generated hydrogen, and any combination thereof. As used herein, the term “external hydrogen” refers to hydrogen that does not originate from the biomass reaction itself, but rather is added to the system from another source.

Another such chemical modification is interesterification. Naturally produced glycerolipids do not have a uniform distribution of fatty acid constituents. In the context of oils, interesterification refers to the exchange of acyl radicals between two esters of different glycerolipids. The interesterification process provides a mechanism by which the fatty acid constituents of a mixture of glycerolipids can be rearranged to modify the distribution pattern. Interesterification is a well-known chemical process, and generally comprises heating (to about 200° C.) a mixture of oils for a period (e.g., 30 minutes) in the presence of a catalyst, such as an alkali metal or alkali metal alkylate (e.g., sodium methoxide). This process can be used to randomize the distribution pattern of the fatty acid constituents of an oil mixture, or can be directed to produce a desired distribution pattern. This method of chemical modification of lipids can be performed on materials provided herein, such as microbial biomass with a percentage of dry cell weight as lipid at least 20%.

Directed interesterification, in which a specific distribution pattern of fatty acids is sought, can be performed by maintaining the oil mixture at a temperature below the melting point of some TAGs which might occur. This results in selective crystallization of these TAGs, which effectively removes them from the reaction mixture as they crystallize. The process can be continued until most of the fatty acids in the oil have precipitated, for example. A directed interesterification process can be used, for example, to produce a product with a lower calorie content via the substitution of longer-chain fatty acids with shorter-chain counterparts. Directed interesterification can also be used to produce a product with a mixture of fats that can provide desired melting characteristics and structural features sought in food additives or products (e.g., margarine) without resorting to hydrogenation, which can produce unwanted trans isomers.

Interesterification of oils produced by the methods described herein can be performed in conjunction with one or more of the methods and/or materials, or to produce products, as reported in the following: U.S. Pat. No. 6,080,853 (Nondigestible fat substitutes); U.S. Pat. No. 4,288,378 (Peanut butter stabilizer); U.S. Pat. No. 5,391,383 (Edible spray oil); U.S. Pat. No. 6,022,577 (Edible fats for food products); U.S. Pat. No. 5,434,278 (Edible fats for food products); U.S. Pat. No. 5,268,192 (Low calorie nut products); U.S. Pat. No. 5,258,197 (Reduce calorie edible compositions); U.S. Pat. No. 4,335,156 (Edible fat product); U.S. Pat. No. 7,288,278 (Food additives or medicaments); U.S. Pat. No. 7,115,760 (Fractionation process); U.S. Pat. No. 6,808,737 (Structural fats); U.S. Pat. No. 5,888,947 (Engine lubricants); U.S. Pat. No. 5,686,131 (Edible oil mixtures); and U.S. Pat. No. 4,603,188 (Curable urethane compositions).

In one embodiment in accordance with the invention, transesterification of the oil, as described above, is followed by reaction of the transesterified product with polyol, as reported in U.S. Pat. No. 6,465,642, to produce polyol fatty acid polyesters. Such an esterification and separation process may comprise the steps as follows: reacting a lower alkyl ester with polyol in the presence of soap; removing residual soap from the product mixture; water-washing and drying the product mixture to remove impurities; bleaching the product mixture for refinement; separating at least a portion of the unreacted lower alkyl ester from the polyol fatty acid polyester in the product mixture; and recycling the separated unreacted lower alkyl ester.

Transesterification can also be performed on microbial biomass with short chain fatty acid esters, as reported in U.S. Pat. No. 6,278,006. In general, transesterification may be performed by adding a short chain fatty acid ester to an oil in the presence of a suitable catalyst and heating the mixture. In some embodiments, the oil comprises about 5% to about 90% of the reaction mixture by weight. In some embodiments, the short chain fatty acid esters can be about 10% to about 50% of the reaction mixture by weight. Non-limiting examples of catalysts include base catalysts, sodium methoxide, acid catalysts including inorganic acids such as sulfuric acid and acidified clays, organic acids such as methane sulfonic acid, benzenesulfonic acid, and toluenesulfonic acid, and acidic resins such as Amberlyst 15. Metals such as sodium and magnesium, and metal hydrides also are useful catalysts.

Another such chemical modification is hydroxylation, which involves the addition of water to a double bond resulting in saturation and the incorporation of a hydroxyl moiety. The hydroxylation process provides a mechanism for converting one or more fatty acid constituents of a glycerolipid to a hydroxy fatty acid. Hydroxylation can be performed, for example, via the method reported in U.S. Pat. No. 5,576,027. Hydroxylated fatty acids, including castor oil and its derivatives, are useful as components in several industrial applications, including food additives, surfactants, pigment wetting agents, defoaming agents, water proofing additives, plasticizing agents, cosmetic emulsifying and/or deodorant agents, as well as in electronics, pharmaceuticals, paints, inks, adhesives, and lubricants. One example of how the hydroxylation of a glyceride may be performed is as follows: fat may be heated, preferably to about 30-50° C. combined with heptane and maintained at temperature for thirty minutes or more; acetic acid may then be added to the mixture followed by an aqueous solution of sulfuric acid followed by an aqueous hydrogen peroxide solution which is added in small increments to the mixture over one hour; after the aqueous hydrogen peroxide, the temperature may then be increased to at least about 60° C. and stirred for at least six hours; after the stirring, the mixture is allowed to settle and a lower aqueous layer formed by the reaction may be removed while the upper heptane layer formed by the reaction may be washed with hot water having a temperature of about 60° C.; the washed heptane layer may then be neutralized with an aqueous potassium hydroxide solution to a pH of about 5 to 7 and then removed by distillation under vacuum; the reaction product may then be dried under vacuum at 100° C. and the dried product steam-deodorized under vacuum conditions and filtered at about 50° to 60° C. using diatomaceous earth.

Hydroxylation of microbial oils produced by the methods described herein can be performed in conjunction with one or more of the methods and/or materials, or to produce products, as reported in the following: U.S. Pat. No. 6,590,113 (Oil-based coatings and ink); U.S. Pat. No. 4,049,724 (Hydroxylation process); U.S. Pat. No. 6,113,971 (Olive oil butter); U.S. Pat. No. 4,992,189 (Lubricants and lube additives); U.S. Pat. No. 5,576,027 (Hydroxylated milk); and U.S. Pat. No. 6,869,597 (Cosmetics).

Hydroxylated glycerolipids can be converted to estolides. Estolides consist of a glycerolipid in which a hydroxylated fatty acid constituent has been esterified to another fatty acid molecule. Conversion of hydroxylated glycerolipids to estolides can be carried out by warming a mixture of glycerolipids and fatty acids and contacting the mixture with a mineral acid, as described by Isbell et al., JAOCS 71(2):169-174 (1994). Estolides are useful in a variety of applications, including without limitation those reported in the following: U.S. Pat. No. 7,196,124 (Elastomeric materials and floor coverings); U.S. Pat. No. 5,458,795 (Thickened oils for high-temperature applications); U.S. Pat. No. 5,451,332 (Fluids for industrial applications); U.S. Pat. No. 5,427,704 (Fuel additives); and U.S. Pat. No. 5,380,894 (Lubricants, greases, plasticizers, and printing inks).

The invention, having been described in detail above, is exemplified in the following examples, which are offered to illustrate, but not to limit, the claimed invention. Other examples of genetically engineering microalgae can be found in WO2008/151149, WO2010/063032, WO2010/063031, WO2011/150410, WO2011/150411, WO2012/061647, WO2012/106560, WO2013/158938, WO 2015/051319, WO2014/176515, and PCT/US2016/024106 which show the engineering of cells to express various lipid biosynthesis pathway enzymes, such as, e.g., those mentioned below.

TABLE 8
Lipid biosynthesis pathway proteins.
3-Ketoacyl ACP synthase
Cuphea hookeriana 3-ketoacyl-ACP synthase (GenBank Acc. No. AAC68861.1),
Cuphea wrightii beta-ketoacyl-ACP synthase II (GenBank Acc. No. AAB37271.1),
Cuphea lanceolata beta-ketoacyl-ACP synthase IV (GenBank Acc. No. CAC59946.1),
Cuphea wrightii beta-ketoacyl-ACP synthase II (GenBank Acc. No. AAB37270.1),
Ricinus communis ketoacyl-ACP synthase (GenBank Acc. No. XP_002516228 ),
Gossypium hirsutum ketoacyl-ACP synthase (GenBank Acc. No. ADK23940.1),
Glycine max plastid 3-keto-acyl-ACP synthase II-A (GenBank Acc No. AAW88763.1),
Elaeis guineensis beta-ketoacyl-ACP synthase II (GenBank Acc. No. AAF26738.2),
Helianthus annuus plastid 3-keto-acyl-ACP synthase I (GenBank Acc. No. ABM53471.1),
Glycine max3-keto-acyl-ACP synthase I (GenkBank Acc. No. NP_001238610.1),
Helianthus annuus plastid 3-keto-acyl-ACP synthase II (GenBank Acc ABI18155.1),
Brassica napus beta-ketoacyl-ACP synthetase 2 (GenBank Acc. No. AAF61739.1),
Perilla frutescens beta-ketoacyl-ACP synthase II (GenBank Acc. No. AAC04692.1),
Helianthus annus beta-ketoacyl-ACP synthase II (GenBank Accession No. ABI18155),
Ricinus communis beta-ketoacyl-ACP synthase II (GenBank Accession No. AAA33872),
Haematococcus pluvialis beta-ketoacyl acyl carrier protein synthase
(GenBank Accession No. HM560033.1),
Jatropha curcasbeta ketoacyl-ACP synthase I (GenBank Accession No. ABJ90468.1),
Populus trichocarpa beta-ketoacyl-ACP synthase I (GenBank Accession No. XP_002303661.1),
Coriandrum sativum beta-ketoacyl-ACP synthetase I (GenBank Accession No. AAK58535.1),
Arabidopsis thaliana 3-oxoacyl-[acyl-carrier-protein] synthase I
(GenBank Accession No. NP_001190479.1),
Vitis vinifera 3-oxoacyl-[acyl-carrier-protein] synthase I (GenBank Accession No. XP_002272874.2)
Fatty acyl-ACP Thioesterases
Umbellularia californica fatty acyl-ACP thioesterase (GenBank Acc. No. AAC49001),
Cinnamomum camphora fatty acyl-ACP thioesterase (GenBank Acc. No. Q39473),
Umbellularia californica fatty acyl-ACP thioesterase (GenBank Acc. No. Q41635),
Myristica fragrans fatty acyl-ACP thioesterase (GenBank Acc. No. AAB71729),
Myristica fragrans fatty acyl-ACP thioesterase (GenBank Acc. No. AAB71730),
Elaeis guineensis fatty acyl-ACP thioesterase (GenBank Acc. No. ABD83939),
Elaeis guineensis fatty acyl-ACP thioesterase (GenBank Acc. No. AAD42220),
Populus tomentosa fatty acyl-ACP thioesterase (GenBank Acc. No. ABC47311),
Arabidopsis thaliana fatty acyl-ACP thioesterase (GenBank Acc. No. NP_172327),
Arabidopsis thaliana fatty acyl-ACP thioesterase (GenBank Acc. No. CAA85387),
Arabidopsis thaliana fatty acyl-ACP thioesterase (GenBank Acc. No. CAA85388),
Gossypium hirsutum fatty acyl-ACP thioesterase (GenBank Acc. No. Q9SQI3),
Cuphea lanceolata fatty acyl-ACP thioesterase (GenBank Acc. No. CAA54060),
Cuphea hookeriana fatty acyl-ACP thioesterase (GenBank Acc. No. AAC72882),
Cuphea calophylla subsp. mesostemon fatty acyl-ACP thioesterase (GenBank Acc. No. ABB71581),
Cuphea lanceolata fatty acyl-ACP thioesterase (GenBank Acc. No. CAC19933),
Elaeis guineensis fatty acyl-ACP thioesterase (GenBank Acc. No. AAL15645),
Cuphea hookeriana fatty acyl-ACP thioesterase (GenBank Acc. No. Q39513),
Gossypium hirsutum fatty acyl-ACP thioesterase (GenBank Acc. No. AAD01982),
Vitis vinifera fatty acyl-ACP thioesterase (GenBank Acc. No. CAN81819),
Garcinia mangostana fatty acyl-ACP thioesterase (GenBank Acc. No. AAB51525),
Brassica juncea fatty acyl-ACP thioesterase (GenBank Acc. No. ABI18986),
Madhuca longifolia fatty acyl-ACP thioesterase (GenBank Acc. No. AAX51637),
Brassica napus fatty acyl-ACP thioesterase (GenBank Acc. No. ABH11710),
B rassica napus fatty acyl-ACP thioesterase (GenBank Acc. No. CAA52070.1),
Oryza sativa (indica cultivar-group) fatty acyl-ACP thioesterase (GenBank Acc. No. EAY86877),
Oryza sativa (japonica cultivar-group) fatty acyl-ACP thioesterase (GenBank Acc. No. NP 001068400),
Oryza sativa (indica cultivar-group) fatty acyl-ACP thioesterase (GenBank Acc. No. EAY99617),
Cuphea hookeriana fatty acyl-ACP thioesterase (GenBank Acc. No. AAC49269),
Ulmus Americana fatty acyl-ACP thioesterase (GenBank Acc. No. AAB71731),
Cuphea lanceolata fatty acyl-ACP thioesterase (GenBank Acc. No. CAB60830),
Cuphea palustris fatty acyl-ACP thioesterase (GenBank Acc. No. AAC49180),
Iris germanica fatty acyl-ACP thioesterase (GenBank Acc. No. AAG43858,
Iris germanica fatty acyl-ACP thioesterase (GenBank Acc. No. AAG43858.1),
Cuphea palustris fatty acyl-ACP thioesterase (GenBank Acc. No. AAC49179),
Myristica fragrans fatty acyl-ACP thioesterase (GenBank Acc. No. AAB71729),
Myristica fragrans fatty acyl-ACP thioesterase (GenBank Acc. No. AAB717291.1),
Cuphea hookeriana fatty acyl-ACP thioesterase GenBank Acc. No. U39834),
Umbelluaria californica fatty acyl-ACP thioesterase (GenBank Acc. No. M94159),
Cinnamomum camphora fatty acyl-ACP thioesterase (GenBank Acc. No. U31813),
Ricinus communis fatty acyl-ACP thioesterase (GenBank Acc. No. ABS30422.1),
Helianthus annuus acyl-ACP thioesterase (GenBank Accession No. AAL79361.1),
Jatropha curcas acyl-ACP thioesterase (GenBank Accession No. ABX82799.3),
Zea mays oleoyl-acyl carrier protein thioesterase, (GenBank Accession No. ACG40089.1),
Haematococcus pluvialis fatty acyl-ACP thioesterase (GenBank Accession No. HM560034.1)
Desaturase Enzymes
Linum usitatissimum fatty acid desaturase 3C, (GenBank Acc. No. ADV92272.1),
Ricinus communis omega-3 fatty acid desaturase,
endoplasmic reticulum, putative, (GenBank Acc. No. EEF36775.1),
Vernicia fordii omega-3 fatty acid desaturase, (GenBank Acc. No. AAF12821),
Glycine max chloroplast omega 3 fatty acid desaturase isoform 2, (GenBank Acc. No. ACF19424.1),
Prototheca moriformis FAD-D omega 3 desaturase (SEQ ID NO: 10),
Prototheca moriformis linoleate desaturase (SEQ ID NO: 11),
Carthamus tinctorius delta 12 desaturase, (GenBank Accession No. ADM48790.1),
Gossypium hirsutum omega-6 desaturase, (GenBank Accession No. CAA71199.1),
Glycine max microsomal desaturase (GenBank Accession No. BAD89862.1),
Zea mays fatty acid desaturase (GenBank Accession No. ABF50053.1),
Brassica napa linoleic acid desaturase (GenBank Accession No. AAA32994.1),
Camelina sativa omega-3 desaturase (SEQ ID NO: 12),
Prototheca moriformis delta 12 desaturase allele 2 (SEQ ID NO: 13,
Camelina sativa omega-3 FAD7-1 (SEQ ID NO: 14),
Helianthus annuus stearoyl-ACP desaturase, (GenBank Accession No. AAB65145.1),
Ricinus communis stearoyl-ACP desaturase, (GenBank Accession No. AACG59946.1),
Brassica juncea plastidic delta-9-stearoyl-ACP desaturase (GenBank Accession No. AAD40245.1),
Glycine max stearoyl-ACP desaturase (GenBank Accession No. ACJ39209.1),
Olea europaea stearoyl-ACP desaturase (GenBank Accession No. AAB67840.1),
Vernicia fordii stearoyl-acyl-carrier protein desaturase, (GenBank Accession No. ADC32803.1),
Descurainia sophia delta-12 fatty acid desaturase (GenBank Accession No. AB586964.2),
Euphorbia lagascae delta12-oleic acid desaturase (GenBank Acc. No. AAS57577.1),
Chlorella vulgaris delta 12 fatty acid desaturase (GenBank Accession No. ACF98528),
Chlorella vulgaris omega-3 fatty acid desaturase (GenBank Accession No. BAB78717),
Haematococcus pluvialis omega-3 fatty acid desaturase (GenBank Accession No. HM560035.1),
Haematococcus pluvialis stearoyl-ACP-desaturase GenBank Accession No. EFS 86860.1,
Haematococcus pluvialis stearoyl-ACP-desaturase GenBank Accession No. EF523479.1
Oleate 12-hydroxylase Enzymes
Ricinus communis oleate 12-hydroxylase (GenBank Acc. No. AAC49010.1),
Physaria lindheimeri oleate 12-hydroxylase (GenBank Acc. No. ABQ01458.1),
Physaria lindheimeri mutant bifunctional oleate 12-hydroxylase:
desaturase (GenBank Acc. No. ACF17571.1),
Physaria lindheimeri bifunctional oleate 12-hydroxylase:
desaturase (GenBank Accession No. ACQ42234.1),
Physaria lindheimeri bifunctional oleate 12-hydroxylase:desaturase (GenBank Acc. No. AAC32755.1),
Arabidopsis lyrata subsp. Lyrata (GenBank Acc. No. XP_002884883.1)
Glycerol-3-phosphate Enzymes
Arabidopsis thaliana glycerol-3-phosphate acyltransferase BAA00575,
Chlamydomonas reinhardtii glycerol-3-phosphate acyltransferase (GenBank Acc. No. EDP02129),
Chlamydomonas reinhardtii glycerol-3-phosphate acyltransferase (GenBank Acc. No. Q886Q7),
Cucurbita moschata acyl-(acyl-carrier-protein):glycerol-3-phosphate
acyltransferase (GenBank Acc. No. BAB39688),
Elaeis guineensis glycerol-3-phosphate acyltransferase, ((GenBank Acc. No. AAF64066),
Garcina mangostana glycerol-3-phosphate acyltransferase (GenBank Acc. No. ABS86942),
Gossypium hirsutum glycerol-3-phosphate acyltransferase (GenBank Acc. No. ADK23938),
Jatropha curcas glycerol-3-phosphate acyltransferase (GenBank Acc. No. ADV77219),
Jatropha curcas plastid glycerol-3-phosphate acyltransferase (GenBank Acc. No. ACR61638),
Ricinus communis plastidial glycerol-phosphate acyltransferase (GenBank Acc. No. EEF43526),
Vica faba glycerol-3-phosphate acyltransferase (GenBank Accession No. AAD05164),
Zea mays glycerol-3-phosphate acyltransferase (GenBank Acc. No. ACG45812)
Lysophosphatidic acid acyltransferase Enzymes
Arabidopsis thaliana 1-acyl-sn-glycerol-3-phosphate acyltransferase (GenBank Accession No. AEE85783),
Brassica juncea 1-acyl-sn-glycerol-3-phosphate acyltransferase (GenBank Accession No. ABQ42862 ),
Brassica juncea 1-acyl-sn-glycerol-3-phosphate acyltransferase (GenBank Accession No. ABM92334),
Brassica napus 1-acyl-sn-glycerol-3-phosphate acyltransferase (GenBank Accession No. CAB09138),
Chlamydomonas reinhardtii lysophosphatidic acid acyltransferase GenBank Accession No. EDP02300),
Cocos nucifera lysophosphatidic acid acyltransferase (GenBank Acc. No. AAC49119),
Limnanthes alba lysophosphatidic acid acyltransferase (GenBank Accession No. EDP02300),
Limnanthes douglasii 1-acyl-sn-glycerol-3-phosphate acyltransferase
(putative) (GenBank Accession No. CAA88620),
Limnanthes douglasii acyl-CoA:sn-1-acylglycerol-3-phosphate acyltransferase
(GenBank Accession No. ABD62751),
Limnanthes douglasii 1-acylglycerol-3-phosphate 0-acyltransferase
(GenBank Accession No. CAA58239),
Ricinus communis 1-acyl-sn-glycerol-3-phosphate acyltransferase (GenBank Accession No. EEF39377)
Diacylglycerol acyltransferase Enzymes
Arabidopsis thaliana diacylglycerol acyltransferase (GenBank Acc. No. CAB45373),
Brassica juncea diacylglycerol acyltransferase (GenBank Acc. No. AAY40784),
Elaeis guineensis putative diacylglycerol acyltransferase (GenBank Acc. No. AEQ94187),
Elaeis guineensis putative diacylglycerol acyltransferase (GenBank Acc. No. AEQ94186),
Glycine max acyl CoA:diacylglycerol acyltransferase (GenBank Acc. No. AAT73629),
Helianthus annus diacylglycerol acyltransferase (GenBank Acc. No. ABX61081),
Olea europaea acyl-CoA:diacylglycerol acyltransferase 1 (GenBank Acc. No. AAS01606),
Ricinus communis diacylglycerol acyltransferase (GenBank Acc. No. AAR11479)
Phospholipid diacylglycerol acyltransferase Enzymes
Arabidopsis thaliana phospholipid:diacylglycerol acyltransferase (GenBank Acc. No. AED91921),
Elaeis guineensis putative phospholipid: diacylglycerol acyltransferase (GenBank Acc. No. AEQ94116),
Glycine max phospholipid:diacylglycerol acyltransferase 1-like (GenBank Acc. No. XP 003541296),
Jatropha curcas phospholipid: diacylglycerol acyltransferase (GenBank Acc. No. AEZ56255),
Ricinus communis phospholipid:diacylglycerol acyltransferase (GenBank Acc. No. ADK92410),
Ricinus communis phospholipid: diacylglycerol acyltransferase (GenBank Acc. No. AEW99982)

### XI. Examples

### Example 1: Fatty Acid Analysis by Fatty Acid Methyl Ester Detection

Lipid samples were prepared from dried biomass. 20-40 mg of dried biomass was resuspended in 2 mL of 5% H₂SO₄ in MeOH, and 200 ul of toluene containing an appropriate amount of a suitable internal standard (C19:0) was added. The mixture was sonicated briefly to disperse the biomass, then heated at 70-75° C. for 3.5 hours. 2 mL of heptane was added to extract the fatty acid methyl esters, followed by addition of 2 mL of 6% K₂CO₃ (aq) to neutralize the acid. The mixture was agitated vigorously, and a portion of the upper layer was transferred to a vial containing Na₂SO₄ (anhydrous) for gas chromatography analysis using standard FAME GC/FID (fatty acid methyl ester gas chromatography flame ionization detection) methods. Fatty acid profiles reported below were determined by this method.

### Example 2: Triacylglyceride Purification from Oil and Methods for Triacylglyceride Lipase Digestion

The triacylglyceride (TAG) fraction of each oil sample was isolated by dissolving ˜10 mg of oil in dichloromethane and loading it onto a Bond-Elut aminopropyl solid-phase extraction cartridge (500 mg) preconditioned with heptane. TAGs were eluted with dicholoromethane-MeOH (1:1) into a collection tube, while polar lipids were retained on the column. The solvent was removed with a stream of nitrogen gas. Tris buffer and 2 mg porcine pancreatic lipase (Type II, Sigma, 100-400 units/mg) were added to the TAG fraction, followed by addition of bile salt and calcium chloride solutions. The porcine pancreatic lipase cleaves sn-1 and sn-3 fatty acids, thereby generating 2-monoacylglycerides and free fatty acids. This mixture was heated with agitation at 40° C. for three minutes, cooled briefly, then quenched with 6 N HCl. The mixture was then extracted with diethyl ether and the ether layer was washed with water then dried over sodium sulfate. The solvent was removed with a stream of nitrogen. To isolate the monoacylglyceride (MAG) fraction, the residue was dissolved in heptane and loaded onto a second aminopropyl solid phase extraction cartridge pretreated with heptane. Residual TAGs were eluted with diethyl ether-dichloromethane-heptane (1:9:40), diacylglycerides (DAGs) were eluted with ethyl acetate-heptane (1:4), and MAGs were eluted from the cartridge with dichloromethane-methanol (2:1). The resulting MAG, DAG, and TAG fractions were then concentrated to dryness with a stream of nitrogen and subjected to routine direct transesterification method of GC/FID analysis as described in Example 1.

### Example 3: Analysis of Regiospecific Profile LC/MS TAG Distribution Analyses were Carried Out Using a Shimadzu

Nexera ultra high performance liquid chromatography system that included a SIL-30AC autosampler, two LC-30AD pumps, a DGU-20A5 in-line degasser, and a CTO-20A column oven, coupled to a Shimadzu LCMS 8030 triple quadrupole mass spectrometer equipped with an APCI source. Data was acquired using a Q3 scan of m/z 350-1050 at a scan speed of 1428 u/sec in positive ion mode with the CID gas (argon) pressure set to 230 kPa. The APCI, desolvation line, and heat block temperatures were set to 300, 250, and 200° C., respectively, the flow rates of the nebulizing and drying gases were 3.0 L/min and 5.0 L/min, respectively, and the interface voltage was 4500 V. Oil samples were dissolved in dichloromethane-methanol (1:1) to a concentration of 5 mg/mL, and 0.8 μL of sample was injected onto Shimadzu Shim-pack XR-ODS III (2.2 μm, 2.0×200 mm) maintained at 30° C. A linear gradient from 30% dichloromethane-2-propanol (1:1)/acetonitrile to 51% dichloromethane-2-propanol (1:1)/acetonitrile over 27 minutes at 0.48 mL/min was used for chromatographic separations.

### Example 4: Preparation of Oil Enriched in Capric Acid

Triglyceride oils typically do not have high capric acid (C10:0) content. Most plant and animal oils have vanishingly small amounts of capric acid, often reported as 0%. The highest capric acid content of commercial oils is coconut oil with about 10% capric acid and palm kernel oil with about 4% capric acid.

Prototheca was engineered to produce capric acid. Recombinant strain A126 produced over 75% capric acid.

Strain A126 was prepared as follows. Base strain S6165 is a non-recombinant, classically mutagenized Prototheca moriformis strain derived from UTEX1435. UTEX 1435 was obtained from the University of Texas culture collection and classically mutagenized to increase lipid yield. The classical mutagenesis did not alter the fatty acid profile of the oil produced by S6165 when compared to UTEX 1435.

Strain A126 was created by two successive transformations of S6165. S6165 was first transformed with construct D3118 (SEQ ID NO:15) by biolistic transformation to prepare strain S7897. Next, S7897 was transformed with construct D3798 (SEQ ID NO:16).

Construct D3118 is written as DAO1b-5′::CrTUB2-ScSUC2-PmPGH:PmSAD2-2p-PmSADtp-CwKASA1-CvNR:PmSAD2-2p-CpSAD1tp_trimmed: CpauFATB1-CvNR::DAO1b-3′. D3118 targets integration into the DAO1b locus via homologous recombination. Proceeding in the 5′ to 3′ direction, the Chlamydomonas reinhardtii β-tubulin promoter (CrTUB2) drives expression of the Saccharomyces cerevisiae sucrose invertase gene (ScSUC2). PmPGH is the Prototheca moriformis PGH3′ UTR. Next, the Prototheca moriformis SAD2-2p promoter (PmSAD2-2p), followed by a Prototheca moriformis SAD transit peptide (PmSADtp) drives the expression of the Cuphea wrightii KASA1 gene (CwKASA1), followed by the Chlorella vulgaris nitrate reductase 3′ UTR (CvNR). Construct D3118 also provides polynucleotides for expression of a Cuphea paucipetala FATB1 (CpauFATB1) driven by the Prototheca moriformis SAD2-2p promoter (PmSAD2-2p) and Chlorella protothecoides SAD1 transit peptide (SAD1tp), and followed by the Chlorella vulgaris nitrate reductase 3′ UTR (CvNR).

Construct D3798 is written as KASI-2ver2_5′::PmHXT1-2v2-ScarMEL1-PmPGK:CvNR:PmSAD2-2v3-PmSADtp-CpauKASIVa-CvNR:PmSAD2-2v3-CpSAD1tp_tr2-CcFATB4-CvNR::KAS1-2ver2_3′. D3798 targets integration into the KAS1 locus thereby knocking out one or both alleles of the endogenous KAS1 gene. Proceeding in the 5′ to 3′ direction, the Prototheca moriformis HXT1-2v2 promoter drives expression of the Saccharomyces carlsbergensis MEL1 gene, conferring the ability to grow on melibiose, and was utilized as the selectable marker. PmPGK is the Prototheca moriformis PGK 3′ UTR and CvNR is the Chlorella vulgaris nitrate reductase 3′ UTR. Next, Prototheca moriformis SAD2-2v3 promoter (PmSAD2-2v3), followed by a Prototheca moriformis SAD transit peptide (PmSADtp) drives the expression of the Cuphea paucipetala KASIVa gene, followed by the Chlorella vulgaris nitrate reductase 3′ UTR (CvNR). Construct D3798 also provides sequences for expression of a Cinnamomum camphora FATB4 (CcFATB4) driven by the Prototheca moriformis SAD2-2v3 promoter (PmSAD2-2v3) and Chlorella protothecoides SAD1 transit peptide (SAD1tp-tr2), and followed by the Chlorella vulgaris nitrate reductase 3′ UTR (CvNR).

The fatty acid profiles of 56165, 57897 and A126 are shown below in Table 9.

TABLE 9

| Strain | C8:0 | C10:0 | C12:0 | C14:0 | C16:0 | C18:0 | C18:1 | C18:2 | C18:3 α |
|---|---|---|---|---|---|---|---|---|---|
| S6165; pH 5 | 0.00 | 0.00 | 0.04 | 1.40 | 29.13 | 3.22 | 58.06 | 5.59 | 0.63 |
| S7897; pH 5 | 0.08 | 17.72 | 2.31 | 3.42 | 23.08 | 1.87 | 43.35 | 5.96 | 0.61 |
| A126; pH 5 | 0.48 | 75.37 | 8.65 | 1.48 | 3.31 | 0.21 | 6.64 | 3.37 | 0.29 |

### Example 5: Preparation of Oil Enriched in Caprylic Acid and Capric Acid

Triglyceride oils typically do not have high caprylic acid (C8:0) and capric acid (C10:0) content. Most plant and animal oils have vanishingly small amounts of caprylic acid and capric acid, often reported as 0%. The highest caprylic acid content of commercial oils is coconut oil with about 9% caprylic acid and palm kernel oil with about 3% caprylic acid. The combined caprylic and capric acid content of coconut oil is less than 20% and for palm coconut oil, it is less than 8%.

Prototheca was engineered to produce both caprylic acid and capric acid. Recombinant strain S8610 produced 21% caprylic acid and 34% capric acid.

Strain S8610 was prepared with base strain S6165. Strain S8610 was created by two successive transformations of S6165. S6165 was first transformed with construct D3104 (SEQ ID NO:17) by biolistic transformation to prepare strain S7786. Next, S7786 was transformed with construct D3937 (SEQ ID NO:18) by biolistic transformation to make strain S8610.

Construct D3104 is written as THI4a::CrTUB2-ScSUC2-PmPGH:PmACP1-1p-CpSAD1tp_ChFATB2ExtC_FLAG-CvNR::THI4a. D3104 targets integration into the THI4A locus via homologous recombination. Proceeding in the 5′ to 3′ direction, the Chlamydomonas reinhardtii β-tubulin promoter (CrTUB2) drives expression of the Saccharomyces cerevisiae sucrose invertase gene (ScSUC2). PmPGH is the Prototheca moriformis PGH3′ UTR. Next, Prototheca moriformis ACP1-1p promoter (PmACP1-1p), followed by a Chlorella protothecoides SAD transit peptide (CpSADtp) drives the expression of the Cuphea hookeriana FATB2gene (ChFATB2), followed by the Chlorella vulgaris nitrate reductase 3′ UTR (CvNR). The THI4 gene encodes an enzyme required for synthesis of thiamine. THI4 catalyzes the synthesis of a thiazole containing moiety, which eventually condenses with a pyrimidine containing moiety to produce thiamine.

Construct D3937 is written as KASI-1ver2_5′::PmHXT1-2v2-ScarMEL1-PmPGK:CvNR:PmSAD2-2v3-PmSADtp-CpauKASIVa-CvNR:PmACP1-1p-CpSAD1tp_trmd:CcFATB4-CvNR::KAS1-1ver2_3′. D3937 targets integration into the KAS1 locus thereby knocking out one or both alleles of the endogenous KAS1 gene. Proceeding in the 5′ to 3′ direction, the Prototheca moriformis HXT1-2v2 promoter drives expression of the Saccharomyces carlsbergensis MEL1 gene, conferring the ability to grow on melibiose, and was utilized as the selectable marker. PmPGK is the Prototheca moriformis PGK3′ UTR and CvNR is the Chlorella vulgaris nitrate reductase 3′ UTR. Next, the Prototheca moriformis SAD2-2v3 promoter (PmSAD2-2v3), followed by a Prototheca moriformis SAD transit peptide (PmSADtp) drives the expression of the Cuphea paucipetala KASIVa gene, followed by the Chlorella vulgaris nitrate reductase 3′ UTR (CvNR). Construct D3937 also provides sequences for expression of a Cinnamomum camphora FATB4 (CcFATB4) driven by the Prototheca moriformis ACP1-1p promoter (PmACP1-1p) and Chlorella protothecoides SAD1 transit peptide (SAD1tp-trmd), and followed by the Chlorella vulgaris nitrate reductase 3′ UTR (CvNR).

The fatty acid profiles of S6165, S7786 and S8610 are shown below in Table 10.

TABLE 10

| Strain | C8:0 | C10:0 | C12:0 | C14:0 | C16:0 | C18:0 | C18:1 | C18:2 | C18:3 α |
|---|---|---|---|---|---|---|---|---|---|
| S6165; pH 7 | 0.00 | 0.00 | 0.00 | 1.56 | 31.06 | 3.40 | 56.02 | 5.78 | 0.61 |
| S7786; pH 7 | 9.21 | 21.53 | 0.47 | 1.36 | 15.89 | 2.46 | 41.44 | 5.60 | 0.59 |
| S8610; pH 7 | 20.76 | 33.76 | 0.76 | 1.39 | 6.51 | 1.08 | 29.30 | 4.77 | 0.38 |

The triacylglycerol profile of S8610 oil is shown in table 11. As used in this application, the abbreviations “Cy” is caprylic, “Ca” is capric, “La” is lauric, “M” is myristic, “P” is palmitic, “S” is stearic, “O” is oleic, “L” is linoleic and “Ln” is linolenic. Table 11 shows that over 50% of the population of TAG molecules in S8610 oil comprise triacylglyceride molecules in which there are two caprylic or capric fatty acids and one palmitic, stearic, oleic, linoleic or linolenic fatty acid on one TAG molecule. Similarly, over 20% of the population of TAG molecules comprise triacylglycerol molecules in which there are two pamitic, stearic, oleic, linoleic or linolenic fatty acids and one caprylic or capric fatty acids on one TAG molecule.

TABLE 11

| Non- | |
|---|---|
| regiospecific | |
| TAG | |
| Profile | Area % |
| CCyCyCy | .60 |
| CCyCyCa | .44 |
| CCyCaCa | .97 |
| CCyLCy | .19 |
| CCaCaCa | .42 |
| CCyCaCa | .03 |
| CCyOCy | .29 |
| CCyCaM | .62 |
| CCyCyP | .75 |
| CCaLCa | .55 |
| CCyOCa | 3.79 |
| CCaCaM | .38 |
| CCyCaP | .23 |
| CCaOCa | 4.60 |
| CCaCaP | .32 |
| CCyOL | .08 |
| CCyOM | .46 |
| CCaOL | .94 |
| CCyOO | .36 |
| CCaOM | .25 |
| CCyOP | .28 |
| CCaOO | .73 |
| CCaOP | .14 |
| TTotal | 8.42 |

### Example 6: Preparation of Oil Enriched in Capric Acid and Lauric Acid

Triglyceride oils typically do not have both high capric acid (C10:0) and lauric acid (C12:0) content. Most plant and animal oils have vanishingly small amounts of capric acid, often reported as 0%. Commercial oils with abundant lauric acid content are coconut oil and palm kernel oil. Other commercial oils typically have lauric acid content of less than 1%. The combined capric and lauric acid content of coconut oil is about 60% and for palm kernel oil, usually less than 60%.

Prototheca moriformis was engineered to produce high levels of capric acid and lauric acid. Recombinant strain S6207 produced a combined capric acid and lauric acid content of over 80%.

Strain S6207 was prepared with base strain S1920. Base strain S1920 is a non-recombinant, classically mutagenized Prototheca moriformis strain derived from UTEX1435. UTEX 1435 was obtained from the University of Texas culture collection and classically mutagenized to increase lipid yield. Strain S6207 was created by two successive transformations of S1920. S1920 was first transformed with construct D725 (SEQ ID NO:19) by biolistic transformation to prepare strain S2655. S2655 was classically mutagenized to increase capric and lauric levels to generate strain S5050. Next, S5050 was transformed with construct D1681 (SEQ ID NO:20) by biolistic transformation to make strain S6207.

Construct D725 is written as SAD2B_5′::CrTUB2-ScSUC2-CpEF1:PmAMT3-PmFADtp_CwFATB2-CvNR:SAD2B_3′. D725 targets integration into the SAD2B locus via homologous recombination. Proceeding in the 5′ to 3′ direction, the Chlamydomonas reinhardtii β-tubulin promoter (CrTUB2) drives expression of the Saccharomyces cerevisiae sucrose invertase gene (ScSUC2) conferring the ability of the cells to grow on sucrose. CpEF1 is the Chlorella protothecoides EF1 3′UTR. Next, the Prototheca moriformis AMT3 promoter (PmAMT3), followed by a Prototheca moriformis FAD transit peptide (PmFADtp) drives the expression of the Cuphea wrightii FATB2 gene (CwFATB2), followed by the Chlorella vulgaris nitrate reductase 3′ UTR (CvNR).

Construct D1681 is written as KAS1-1_5′::CrTUB2-NeoR-CvNR:PmUAPA1-ChFATB2-CpCD181:PmAMT3-PmSADtp-CwKASA1-CvNR::KAS1-1_3′. D1681 targets integration into the KAS1-1 locus via homologous recombination thereby knocking out one or both alleles of the endogenous KAS1 gene. Proceeding in the 5′ to 3′ direction, the C. reinhardtii β-tubulin promoter (CrTUB2) drives expression of the neomycin phosphotransferase gene (NeoR) conferring the ability of the cells to grow on G418. CvNR is the Chlorella vulgaris nitrate reductase 3′UTR. Next, the Prototheca moriformis UAPA1 promoter (PmUAPA1) drives the expression of the Cuphea hookeriana FATB2 gene (ChFATB2). CpCD181 is the Chlorellaprotothecoides CD181 3′UTR. Next, the Prototheca moriformis AMT3 promoter (PmAMT3) and the Prototheca moriformis SAD transit peptide (PmSADtp) drive expression of the Cuphea wrightii KASA1, followed by the Chlorella vulgaris nitrate reductase 3′ UTR (CvNR).

The fatty acid profiles of S1920, S2655, S5050, and S6207 are shown below in Table 12.

TABLE 12

| Strain | C8:0 | C10:0 | C12:0 | C14:0 | C16:0 | C18:0 | C18:1 | C18:2 | C18:3 α |
|---|---|---|---|---|---|---|---|---|---|
| S1920; pH 7 | 0.00 | 0.01 | 0.04 | 1.45 | 30.27 | 3.73 | 57.50 | 5.36 | 0.33 |
| S2655; pH 7 | 0.03 | 3.89 | 20.22 | 11.90 | 20.96 | 1.84 | 34.60 | 5.11 | 0.47 |
| S5050; pH 7 | 0.17 | 14.81 | 43.26 | 16.26 | 11.03 | 0.61 | 9.96 | 2.75 | 0.60 |
| S6207; pH 7 | 0.97 | 37.07 | 47.11 | 4.08 | 2.31 | 0.25 | 5.26 | 1.74 | 0.28 |

### Example 7: Hydrogenation of Oil Enriched in Caprylic Acid and Capric Acid

The oil of Example 6 enriched in C8:0 and C10:0 was hydrogenated in a 2 L Parr reactor using 0.5% Pricat Ni 62/15P catalyst at a temperature of 155° C. using hydrogen at a pressure of 50PSI to completely hydrogenate the oil. Pricat NI 62/15P is a commercially available catalyst containing Ni and NiO phases on mixed supports silica, magnesia and graphite. The reaction was carried out for about 60 minutes and the iodine value of the fully hydrogenated oil was less than 1, indicating complete hydrogenation. Hydrogenated oils with iodine values of less than 4 are deemed to be fully hydrogenated by the FDA. Hydrogenation converts unsaturated fatty acid to saturated fatty acids, for example, converting oleic acid to stearic acid.

Table 13 below shows the fatty acid composition of hydrogenated oil of Example 6. The data show that the unsaturated fatty acids C18:1, C18:2 and C18:3 have been hydrogenated and converted to C18:0. The amounts of all other saturated fatty acids, with the exception of C18:0 remained constant. There is a slight decrease in the C8:0 content, but this is due to losses during processing of the hydrogenated oil.

TABLE 13

| Strain | C8:0 | C10:0 | C12:0 | C14:0 | C16:0 | C18:0 | C18:1 | C18:2 | C18:3 α |
|---|---|---|---|---|---|---|---|---|---|
| S6165; pH 7 | 0.00 | 0.00 | 0.00 | 1.56 | 31.06 | 3.40 | 56.02 | 5.78 | 0.61 |
| S8610; pH 7 | 20.76 | 33.76 | 0.76 | 1.39 | 6.51 | 1.08 | 29.30 | 4.77 | 0.38 |
| Hydrogenated | 18.33 | 32.36 | 1.13 | 1.20 | 5.41 | 40.58 | 0.07 | 0.01 | N/A |
| S8610 | | | | | | | | | |

The non-regiospecific triacylglycerol profile of hydrogenated S8610 oil is shown in table 14. Table 14 shows that about 45% of the population of TAG molecules in S8610 oil comprise triacylglyceride molecules in which there are two caprylic or capric fatty acids and one palmitic or stearic fatty acid on one TAG molecule. The hydrogenation converts TAG molecules that contain two caprylic or capric fatty acids and one oleic, linoleic or linolenic acid, the unsaturated fatty acid has been converted to stearic acid. About 30% of the population of TAG molecules comprise triacylglycerol molecules in which there are two palmitic or stearic fatty acids and one caprylic or capric fatty acid on a TAG molecule. In TAG molecules that contain one caprylic or capric fatty acid moiety and one or more oleic acid moieties, the oleic acid moieties have been converted to stearic acid.

TABLE 14

| Non- | |
|---|---|
| regiospecific | Hydrogenated |
| TAG Profile | RBD819 |
| CyCyCy | 0.53 |
| CyCyCa | 3.49 |
| CyCaCa | 6.59 |
| CaCaCa | 3.68 |
| CaCaLa | 0.23 |
| CyCaM | 0.44 |
| CyCyP | 0.61 |
| CaCaM | 0.52 |
| CyCaP | 2.61 |
| CyCyS | 6.60 |
| CaCaP | 1.81 |
| CaCyS | 20.47 |
| CaCaS | 13.94 |
| MMLa + CaMP | 0.86 |
| CyMS | 0.74 |
| LaLaS + LaMP + MMM | 0.90 |
| CyPS | 3.56 |
| CaPS | 4.18 |
| CySS | 10.71 |
| CaSS | 11.56 |
| SSP | 1.10 |
| SSS | 3.00 |
| Total | 98.13 |

### Example 8: Differential Scanning Calorimetry of Non-Hydrogenated Oil

Non-hydrogenated and hydrogenated S8610 Oils were analyzed by differential scanning calorimetry (DSC). The DSC experiments were performed with the following heating and cooling profile. The samples were heated from 30.00° C. to 80.00° C. at 1.00° C. per minute then held for 30.0 minutes at 80.00° C. Next the samples were cooled from 80.00° C. to −65.00° C. at 1.00° C. per minute. When the samples reached −65.00° C. they were held at −65.00° C. for 30.0 min. Next, the samples were heated from −65.00° C. to 80.00° C. at 1.00° C. per minute.

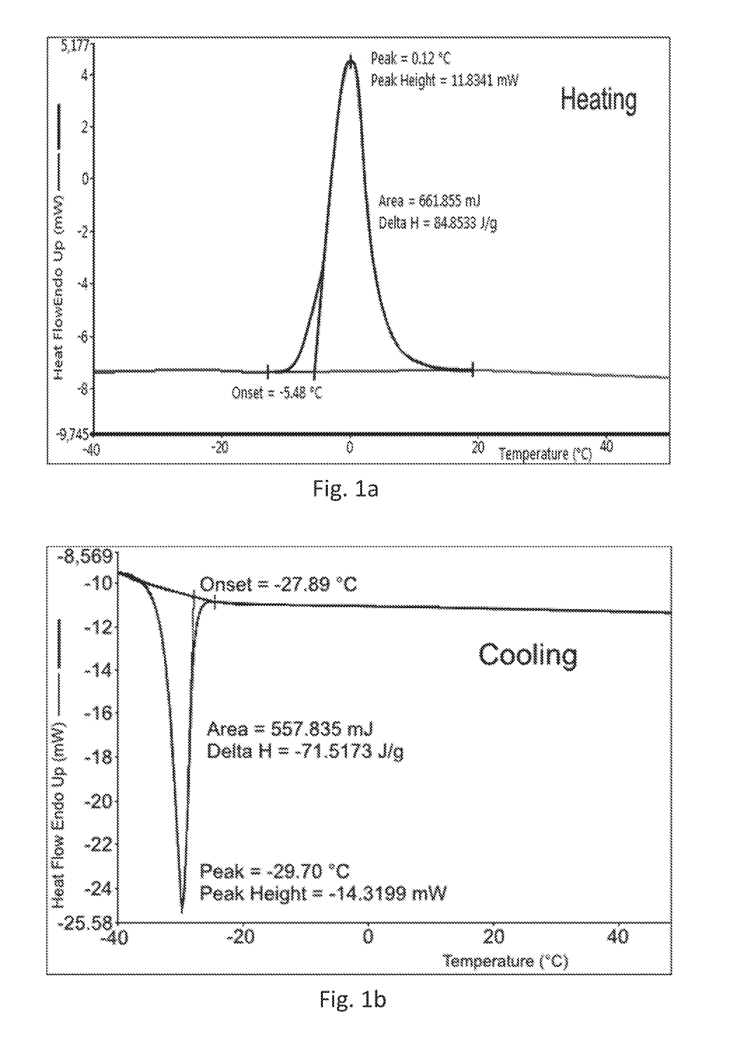

FIG. 1a is the heating curve of non-hydrogenated S8610 oil and FIG. 1b is the cooling curve of non-hydrogenated S8610 oil. The heating curve shows that the non-hydrogenated oil has a wide, single peak having a melting temperature centered at 0.12° C. The cooling curve shows a wide, single peak having a freezing temperature centered at −29.70° C.

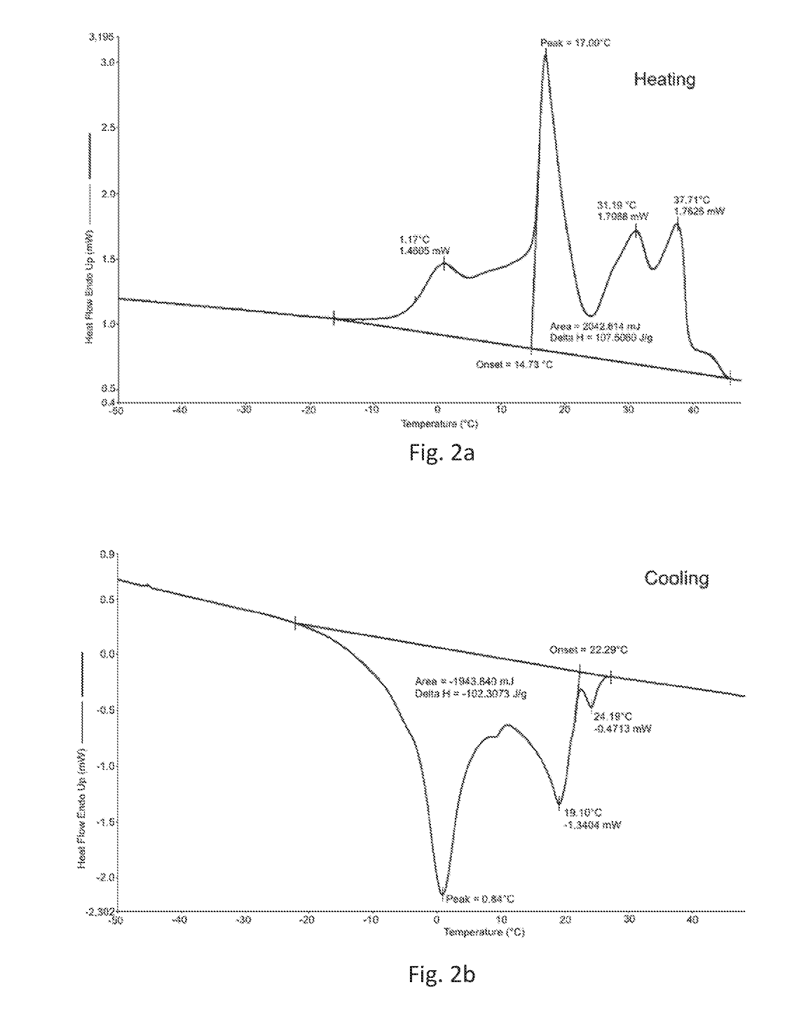

FIG. 2a is the heating curve of hydrogenated S8610 oil and FIG. 2b is the cooling curve of hydrogenated S8610 oil. The heating and cooling curves show that the hydrogenated oil has multiple melting and cooling peaks indicating that multiple populations of triacylglycerides are present. The heating curve shows at least four peaks having melting temperatures centered at 1.17° C., 17.00° C., 31.19° C., and 37.71° C. The triacylglyceride populations that melt at 31.19° C., and 37.71° C. are useful as confectionary fats because these melting temperatures are similar to the temperature of the human mouth. Fats that melt at human mouth temperatures are used as cocoa butter equivalents. The cooling curve shows at least three peaks having freezing temperatures centered at 24.19° C., 19.10° C., and 0.84° C. There appears to be a fourth peak, a shoulder, at about 10° C.

### Example 9: Fractionation of Hydrogenated S8610 Oils

Hydrogenated S8610 oil was fractionated by short path distillation at 180° C., 190° C., 200° C., 210° C., and 220° C. to separate the populations of asymmetric triacylglyceride molecules.

Table 15 shows the TAG profiles of the distillate fraction and the residue fraction of the hydrogenated S8610 oil fractionated at 210° C. The distillate fraction is enriched in triacylglyceride molecules in which there are two caprylic or capric fatty acids and one palmitic or stearic fatty acid. For example, in the distillate fraction, 10.94% of the TAG molecules possess two capric moieties and one stearic moiety but in the the residue fraction, the fraction drops to 0.75%. The residue fraction is enriched in triacylglyceride molecules in which there are two palmitic or stearic fatty acids and one caprylic or capric fatty acid on a TAG molecule. For example, in the residue fraction, 23.92% of the TAG molecules possess one capric moiety and two stearic moities.

TABLE 15

| | Distillate- | Residue- |
|---|---|---|
| | Hydrogenated | Hydrogenated |
| Non- | RBD | RBD |
| regiospecific | Algal oil | Algal oil |
| TAG | (RBD819 high | (RBD819 high |
| Profile | C8/C10)-2-rep1 | C8/C10)-210C-rep1 |
| CyCyCy | 0.81 | N.D. |
| CyCyCa | 6.07 | N.D. |
| CyCaCa | 12.11 | N.D. |
| CaCaCa | 6.70 | N.D. |
| CaCaLa | 0.54 | N.D. |
| CyCaM | 0.91 | N.D. |
| CyCyP | 1.20 | N.D. |
| CaCaM + LaLaCa | 0.88 | N.D. |
| CyCaP | 4.43 | 0.23 |
| CyCyS | 10.94 | 0.75 |
| CaCaP | 2.81 | 0.64 |
| CaCyS | 29.78 | 8.77 |
| CaCaS | 14.54 | 12.88 |
| MMLa + CaMP + LaLaP | 0.56 | 1.24 |
| CyMS | 0.52 | 0.99 |
| MMM + LaMP + LaLaS | 0.46 | 1.95 |
| CySP | 1.47 | 6.33 |
| CaPS | 0.88 | 8.19 |
| CySS | 2.31 | 21.30 |
| CaSS | 1.23 | 23.92 |
| LaPA + MPS | N.D. | 1.17 |
| PPS + SSM | N.D. | 0.99 |
| SSP | N.D. | 2.50 |
| SSS | N.D. | 7.16 |
| SSA | N.D. | 0.27 |
| Total | 99.15 | 99.28 |

N.D.: Not detected

### Example 10: Differential Scanning Calorimetry of Hydrogenated, Fractionated Oil

The hydrogenated, fractionated high caprylic/capric oil of Example X+4 was analyzed by differential scanning calorimetry. The DSC experiments were performed according to the heating and cooling profiles of Example 8.

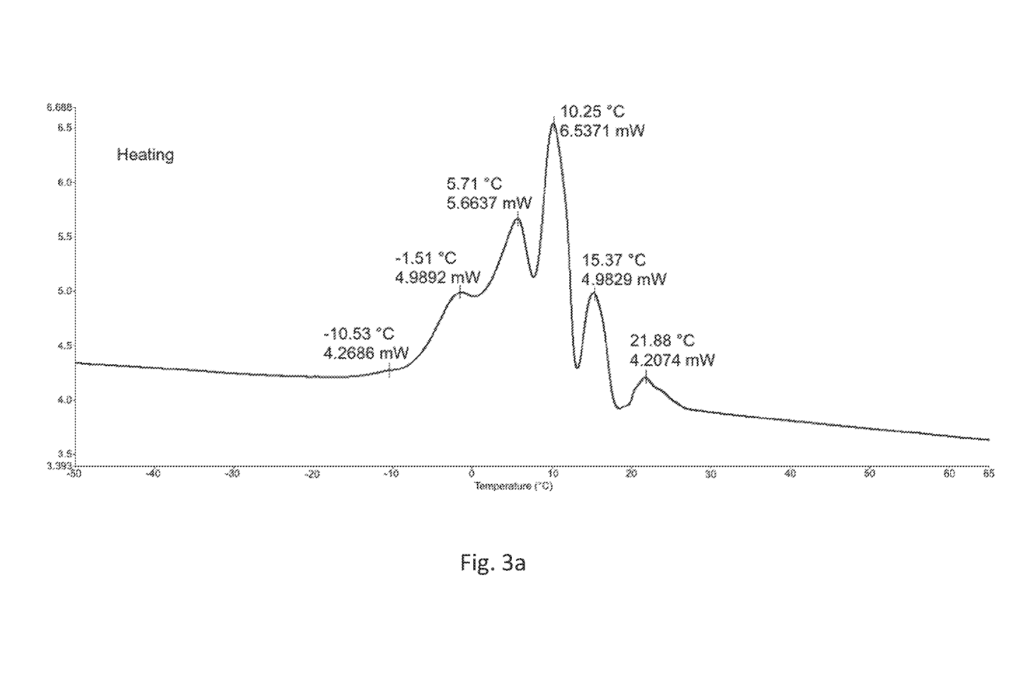

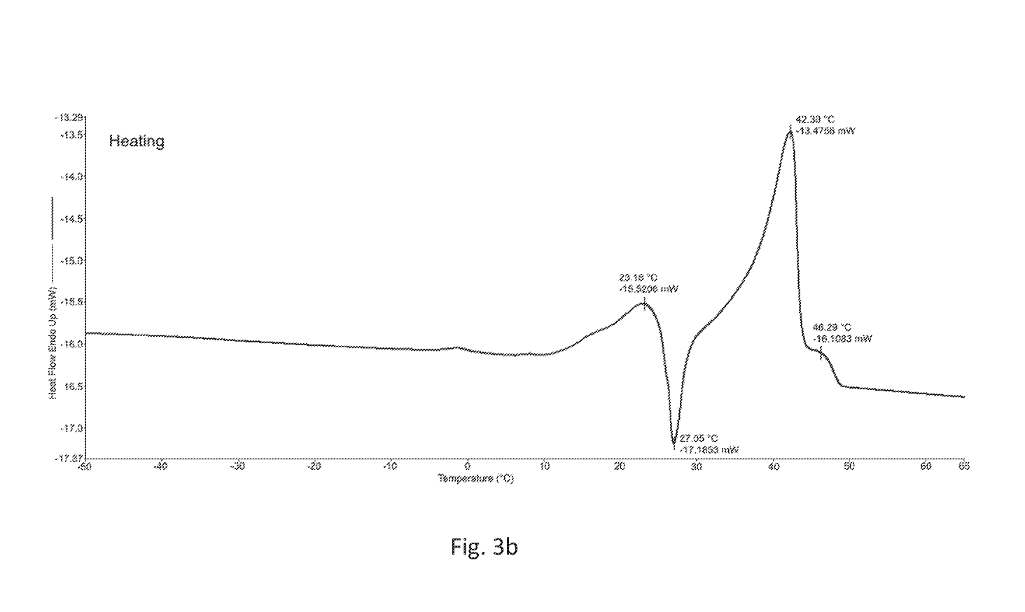

FIG. 3a is the heating curve of distillate fraction of the hydrogenated S8610 oil and FIG. 3b is the cooling curve of residue fraction the hydrogenated S8610 oil. The heating and cooling curves show that the hydrogenated oil has multiple melting and cooling peaks indicating that multiple populations of triacylglycerides are present. The heating curve shows at least five peaks having melting temperatures centered at −10.53° C., 1.51° C., 5.71° C., 10.25° C., 15.37° C., and 21.88° C. The heating curve of the distillate fraction shows an enrichment of TAG populations with lower melting points. The heating curve of the residue fraction shows at least four peaks having melting temperatures centered at 46.29° C., 42.30° C., 27.05° C., and 23.18° C. The heating curve of the residue fraction shows an enrichment of TAG populations with higher melting points. The triacylglyceride populations that melt at higher temperatures near the temperature of the human mouth are useful as confectionary fats. Fats that melt at human mouth temperatures are used as cocoa butter equivalents.

SEQUENCE LISTING

SEQ ID NO: 1
23S rRNA for UTEX 1439, UTEX 1441, UTEX 1435, UTEX 1437
Prototheca moriformis
TGTTGAAGAATGAGCCGGCGACTTAAAATAAATGGCAGGCTAAGAGAATTAATAACTCGAAA
CCTAAGCGAAAGCAAGTCTTAATAGGGCGCTAATTTAACAAAACATTAAATAAAATCTAAAG
TCATTTATTTTAGACCCGAACCTGAGTGATCTAACCATGGTCAGGATGAAACTTGGGTGACA
CCAAGTGGAAGTCCGAACCGACCGATGTTGAAAAATCGGCGGATGAACTGTGGTTAGTGGTG
AAATACCAGTCGAACTCAGAGCTAGCTGGTTCTCCCCGAAATGCGTTGAGGCGCAGCAATAT
ATCTCGTCTATCTAGGGGTAAAGCACTGTTTCGGTGCGGGCTATGAAAATGGTACCAAATCG
TGGCAAACTCTGAATACTAGAAATGACGATATATTAGTGAGACTATGGGGGATAAGCTCCAT
AGTCGAGAGGGAAACAGCCCAGACCACCAGTTAAGGCCCCAAAATGATAATGAAGTGGTAAA
GGAGGTGAAAATGCAAATACAACCAGGAGGTTGGCTTAGAAGCAGCCATCCTTTAAAGAGTG
CGTAATAGCTCACTG
SEQ ID NO: 2
Mature native Protheca moriformis KASII amino acid sequence
(native transit peptide is underlined)
AAAAADANPARPERRVVITGQGVVTSLGQTIEQFYSSLLEGVSGISQIQKFDTTGYTTTIAG
EIKSLQLDPYVPKRWAKRVDDVIKYVYIAGKQALESAGLPIEAAGLAGAGLDPALCGVLIGT
AMAGMTSFAAGVEALTRGGVRKMNPFCIPFSISNMGGAMLAMDIGFMGPNYSISTACATGNY
CILGAADHIRRGDANVMLAGGADAAIIPSGIGGFIACKALSKRNDEPERASRPWDADRDGFV
MGEGAGVLVLEELEHAKRRGATILAELVGGAATSDAHHMTEPDPQGRGVRLCLERALERARL
APERVGYVNAHGTSTPAGDVAEYRAIRAVIPQDSLRINSTKSMIGHLLGGAGAVEAVAAIQA
LRTGWLHPNLNLENPAPGVDPVVLVGPRKERAEDLDVVLSNSFGFGGHNSCVIFRKYDE
SEQ ID NO: 3
Codon-optimized coding region of Brassica napus C18:0-
preferring thioesterase from pSZ1358
ACTAGTATGCTGAAGCTGTCCTGCAACGTGACCAACAACCTGCACACCTTCTCCTTCTTCTC
CGACTCCTCCCTGTTCATCCCCGTGAACCGCCGCACCATCGCCGTGTCCTCCGGGCGCGCCT
CCCAGCTGCGCAAGCCCGCCCTGGACCCCCTGCGCGCCGTGATCTCCGCCGACCAGGGCTCC
ATCTCCCCCGTGAACTCCTGCACCCCCGCCGACCGCCTGCGCGCCGGCCGCCTGATGGAGGA
CGGCTACTCCTACAAGGAGAAGTTCATCGTGCGCTCCTACGAGGTGGGCATCAACAAGACCG
CCACCGTGGAGACCATCGCCAACCTGCTGCAGGAGGTGGCCTGCAACCACGTGCAGAAGTGC
GGCTTCTCCACCGACGGCTTCGCCACCACCCTGACCATGCGCAAGCTGCACCTGATCTGGGT
GACCGCCCGCATGCACATCGAGATCTACAAGTACCCCGCCTGGTCCGACGTGGTGGAGATCG
AGACCTGGTGCCAGTCCGAGGGCCGCATCGGCACCCGCCGCGACTGGATCCTGCGCGACTCC
GCCACCAACGAGGTGATCGGCCGCGCCACCTCCAAGTGGGTGATGATGAACCAGGACACCCG
CCGCCTGCAGCGCGTGACCGACGAGGTGCGCGACGAGTACCTGGTGTTCTGCCCCCGCGAGC
CCCGCCTGGCCTTCCCCGAGGAGAACAACTCCTCCCTGAAGAAGATCCCCAAGCTGGAGGAC
CCCGCCCAGTACTCCATGCTGGAGCTGAAGCCCCGCCGCGCCGACCTGGACATGAACCAGCA
CGTGAACAACGTGACCTACATCGGCTGGGTGCTGGAGTCCATCCCCCAGGAGATCATCGACA
CCCACGAGCTGCAGGTGATCACCCTGGACTACCGCCGCGAGTGCCAGCAGGACGACATCGTG
GACTCCCTGACCACCTCCGAGATCCCCGACGACCCCATCTCCAAGTTCACCGGCACCAACGG
CTCCGCCATGTCCTCCATCCAGGGCCACAACGAGTCCCAGTTCCTGCACATGCTGCGCCTGT
CCGAGAACGGCCAGGAGATCAACCGCGGCCGCACCCAGTGGCGCAAGAAGTCCTCCCGCATG
GACTACAAGGACCACGACGGCGACTACAAGGACCACGACATCGACTACAAGGACGACGACGA
CAAGTGAATCGAT
SEQ ID NO: 4
Brassica napus acyl-ACP thioesterase (Genbank Accession No.
CAA52070) with 3X FLAG Tag (bold)
MLKLSCNVINNLHTFSFFSDSSLFIPVNRRTIAVSS [symbol] SQLRKPALDPLRAVISADQGSIS
PVNSCTPADRLRAGRLMEDGYSYKEKFIVRSYEVGINKTATVETIANLLQEVACNHVQKCGF
STDGFATTLTMRKLHLIWVTARMHIETYKYPAWSDVVEIETWCQSEGRIGTRRDWILRDSAT
NEVIGRATSKWVMMNQDTRRLQRVTDEVRDEYLVFCPREPRLAFPEENNSSLKKIPKLEDPA
QYSMLELKPRRADLDMNQHVNNVTYIGWVLESIPQEIIDTHELQVITLDYRRECQQDDIVDS
LTTSEIPDDPISKFTGTNGSAMSSIQGHNESQFLHMLRLSENGQEINRGRTQWRKKSSRMDY
KDHDGDYKDHDIDYKDDDDK
SEQ ID NO: 5
Brassica napus acyl-ACP thioesterase (GenBank Accession No.
CAA52070) with UTEX 250 stearoyl-ACP desaturase (SAD)
chloroplast transit peptide and 3X FLAG ® Tag
MATASTFSAFNARCGDLRRSAGSGPRRPARPLPVR [symbol] SQLRKPALDPLRAVISADQGSISP
VNSCTPADRLRAGRLMEDGYSYKEKFIVRSYEVGINKTATVETIANLLQEVACNHVQKCGFS
TDGFATTLTMRKLHLIWVTARMHIEIYKYPAWSDVVEIETWCQSEGRIGTRRDWILRDSATN
EVIGRATSKWVMMNQDTRRLQRVTDEVRDEYLVFCPREPRLAFPEENNSSLKKIPKLEDPAQ
YSMLELKPRRADLDMNQHVNNVTYIGWVLESIPQEIIDTHELQVITLDYRRECQQDDIVDSL
TTSEIPDDPISKFTGTNGSAMSSIQGHNESQFLHMLRLSENGQEINRGRTQWRKKSSRMDYK
DHDGDYKDHDIDYKDDDDK
SEQ ID NO: 6
C. tinctorius FATA (GenBank Accession No. AAA33019) with UTEX
250 stearoyl-ACP desaturase (SAD) chloroplast transit peptide
MATASTFSAFNARCGDLRRSAGSGPRRPARPLPVR [symbol] ATGEQPSGVASLREADKEKSLGNR
LRLGSLTEDGLSYKEKFVIRCYEVGINKTATIETIANLLQEVGGNHAQGVGFSTDGFATTTT
MRKLHLIWVTARMHIEIYRYPAWSDVIEIETWVQGEGKVGTRRDWILKDYANGEVIGRATSK
WVMMNEDTRRLQKVSDDVREEYLVFCPRTLRLAFPEENNNSMKKIPKLEDPAEYSRLGLVPR
RSDLDMNKHVNNVTYIGWALESIPPEIIDTHELQAITLDYRRECQRDDIVDSLTSREPLGNA
AGVKFKEINGSVSPKKDEQDLSRFMHLLRSAGSGLEINRCRTEWRKKPAKRMDYKDHDGDYK
DHDIDYKDDDDK
SEQ ID NO: 7
R. communis FATA (Genbank Accession No. ABS30422) with a
3xFLAG ® epitope tag
MLKVPCCNATDPIQSLSSQCRFLTHFNNRPYFTRRPSIPTFFSSKNSSASLQAVVSDISSVE
SAACDSLANRLRLGKLTEDGFSYKEKFIV [symbol] RSYEVGINKTATVETIANLLQEVGCNHAQS
VGFSTDGFATTTSMRKMHLIWVTARMHIEIYKYPAWSDVVEVETWCQSEGRIGTRRDWILTD
YATGQIIGRATSKWVMMNQDTRRLQKVTDDVREEYLVECPRELRLAFPEENNRSSKKISKLE
DPAQYSKLGLVPRRADLDMNQHVNNVTYIGWVLESIPQEIIDTHELQTITLDYRRECQHDDI
VDSLTSVEPSENLEAVSELRGTNGSATTTAGDEDCRNFLHLLRLSGDGLEINRGRTEWRKKS
ARMDYKDHDGDYKDHDIDYKDDDDK
SEQ ID NO: 8
Theobroma cacao FATA1 with 3X FLAG ® epitope tag
MLKLSSCNVTDQRQALAQCRFLAPPAPFSFRWRTPVVVSCSPSSRPNLSPLQVVLSGQQQAG
MELVESGSGSLADRLRLGSLTEDGLSYKEKFIVRCYEVGINKTATVETIANLLQEVGCNHAQ
SVGYSTDGFATTRTMRKLHLIWVTARMHIETYKYPAWSDVIEIETWCQSEGRIGTRRDWILK
DFGTGEVIGRATSKWVMMNQDTRRLQKVSDDVREEYLVFCPRELRLAFPEENNNSLKKIAKL
DDSFQYSRLGLMPRRADLDMNQHVNNVTYIGWVLESMPQEIIDTHELQTITLDYRRECQQDD
VVDSLTSPEQVEGTEKVSAIHGTNGSAAAREDKQDCRQFLHLLRLSSDGQEINRGRTEWRKK
PARMDYKDHDGDYKDHDIDYKDDDDK
SEQ ID NO: 9
G. mangostana FATA1 (GenBank Accession No. AAB51523) with 3X
FLAG ® epitope tag
MLKLSSSRSPLARIPTRPRPNSIPPRIIVVSSSSSKVNPLKTEAVVSSGLADRLRLGSLTED
GLSYKEKFIVRCYEVGINKTATVETIANLLQEVGCNHAQSVGYSTGGFSTTPTMRKLRLIWV
TARMHIEIYKYPAWSDVVEIESWGQGEGKIGTRRDWILRDYATGQVIGRATSKWVMMNQDTR
RLQKVDVDVRDEYLVHCPRELRLAFPEENNSSLKKISKLEDPSQYSKLGLVPRRADLDMNQH
VNNVTYIGWVLESMPQEIIDTHELQTITLDYRRECQHDDVVDSLTSPEPSEDAEAVFNHNGT
NGSANVSANDHGCRNFLHLLRLSGNGLEINRGRTEWRKKPTRMDYKDHDGDYKDHDIDYKDD
DDK
SEQ ID NO: 10
Prototheca moriformis FAD-D omega 3 desaturase
MSIQFALRAAYIKGTCQRLSGRGAALGLSRDWTPGWTLPRCWPASAAATAPPRARHQERAIH
LTSGRRRHSALASDADERALPSNAPGLVMASQANYFRVRLLPEQEEGELESWSPNVRHTTLL
CKPRAMLSKLQMRVMVGDRVIVTAIDPVNMTVHAPPFDPLPATRFLVAGEAADMDITVVLNK
ADLVPEEESAALAQEVASWGPVVLTSTLTGRGLQELERQLGSTTAVLAGPSGAGKSSIINAL
ARAARERPSDASVSNVPEEQVVGEDGRALANPPPFTLADIRNAIPKDCFRKSAAKSLAYLGD
LSITGMAVLAYKINSPWLWPLYWFAQGTMFWALFVVGHDCGHQSFSTSKRLNDALAWLGALA
AGTWTWALGVLPMLNLYLAPYVWLLVTYLHHHGPSDPREEMPWYRGREWSYMRGGLTTIDRD
YGLFNKVHHDIGTHVVHH
SEQ ID NO: 11
MFWALFVVGHDCGHQSFSTSKRLNDAVGLFVHSIIGVPYHGWRISHRTHHNNHGHVENDESW
YPPTESGLKAMTDMGRQGRFHFPSMLFVYPFYLFWRSPGKTGSHFSPATDLFALWEAPLIRT
SNACQLAWLGALAAGTWALGVLPMLNLYLAPYVISVAWLDLVTYLHHHGPSDPREEMPWYRG
REWSYMRGGLTTIDRDYGLFNKVHHDIGTHVVHHLFPQIPHYNLCRATKAAKKVLGPYYREP
ERCPLGLLPVHLLAPLLRSLGQDHFVDDAGSVLFYRRAEGINPWIQKLLPWLGGARRGADAQ
RDAAQ
SEQ ID NO: 12
Camelina sativa omega-3 FAD7-2
MANLVLSECGIRPLPRIYTTPRSNFVSNNNKPIFKFRPFTSYKTSSSPLACSRDGFGKNWSL
NVSVPLTTTTPIVDESPLKEEEEEKQRFDPGAPPPFNLADIRAAIPKHCWVKNPWKSMSYVL
RDVAIVFALAAGASYLNNWIVWPLYWLAQGTMFWALFVLGHDCGHGSFSNNPRLNNVVGHLL
HSSILVPYHGWRISHRTHHQNHGHVENDESWHPMSEKIYQSLDKPTRFFRFTLPLVMLAYPF
YLWARSPGKKGSHYHPESDLFLPKEKTDVLTSTACWTAMAALLICLNFVVGPVQMLKLYGIP
YWINVMWLDFVTYLHHHGHEDKLPWYRGKEWSYLRGGLTTLDRDYGVINNIHHDIGTHVIHH
LFPQIPHYHLVEATEAVKPVLGKYYREPDKSGPLPLHLLGILAKSIKEDHYVSDEGDVVYYK
ADPNMYGEIKVGAD
SEQ ID NO: 13
Prototheca moriformis delta 12 desaturase allele 2
MAIKTNRQPVEKPPFTIGTLRKAIPAHCFERSALRSSMYLAFDIAVMSLLYVASTYIDPAPV
PTWVKYGIMWPLYWFFQGAFGTGVWVCAHECGHQAFSSSQAINDGVGLVFHSLLLVPYYSWK
HSHRRHHSNTGCLDKDEVFVPPHRAVAHEGLEWEEWLPIRMGKVLVILTLGWPLYLMFNVAS
RPYPRFANHFDPWSPIFSKRERIEVVISDLALVAVLSGLSVLGRTMGWAWLVKTYVVPYMIV
NMWLVLITLLQHTHPALPHYFEKDWDWLRGAMATVDRSMGPPFMDSILHHISDTHVLHHLFS
TIPHYHAEEASAAIRPILGKYYQSDSRWVGRALWEDWRDCRYVVPDAPEDDSALWFHK
SEQ ID NO: 14
Camelina sativa omega-3 FAD7-1
MANLVLSECGIRPLPRIYTTPRSNFVSNNNKPIFKFRPLTSYKTSSPLFCSRDGFGRNWSLN
VSVPLATTTPIVDESPLEEEEEEEKQRFDPGAPPPFNLADIRAAIPKHCWVKNPWKSMSYVL
RDVAIVFALAAGAAYLNNWIVWPLYWLAQGTMFWALFVLGHDCGHGSFSNNPRLNNVVGHLL
HSSILVPYHGWRISHRTHHQNHGHVENDESWHPMSEKIYQSLDKPTRFFRFTLPLVMLAYPF
YLWARSPGKKGSHYHPESDLFLPKEKTDVLTSTACWTAMAALLICLNFVVGPVQMLKLYGIP
YWINVMWLDFVTYLHHHGHEDKLPWYRGKEWSYLRGGLTTLDRDYGVINNIHHDIGTHVIHH
LFPQIPHYHLVEATEAVKPVLGKYYREPDKSGPLPLHLLGILAKSIKEDHYVSDEGDVVYYK
ADPNMYGEIKVGAD
SEQ ID NO: 15
D3118/pSZ4354 Sequence
Construct D3118 is written as DAO1b-5′::CrTUB2-ScSUC2-
PmPGH:PmSAD2-2p-PmSADtp-CwKASA1-CvNR:PmSAD2-2p-
CpSAD1tp_trimmed:CpauFATB1-CvNR::DAO1b-3′
agcccgcaccctcgttgatctgggagccctgcgcagccccttaaatcatctcagtcaggttt
ctgtgttcaactgagcctaaagggctttcgtcatgcgcacgagcacacgtatatcggccacg
cagtttctcaaaagcggtagaacagttcgcgagccctcgtaggtcgaaaacttgcgccagta
ctattaaattaaattaattgatcgaacgagacgcgaaacttttgcagaatgccaccgagttt
gcccagagaatgggagtggcgccattcaccatccgcctgtgcccggcttgattcgccgagac
gatggacggcgagaccagggagcggcttgcgagccccgagccggtagcaggaacaatgatcg
acaatcttcctgtccaattactggcaaccattagaaagagccggagcgcgttgaaagtctgc
aatcgagtaatttttcgatacgtcgggcctgctgaaccctaaggctccggactttgtttaag
gcgatccaagatgcacgcggccccaggcacgtatctcaagcacaaaccccagccttagtttc
gagactttgggagatagcgaccgatatctagtttggcattttgtatattaattacctcaagc
aatggagcgctctgatgcggtgcagcgtcggctgcagcacctggcagtggcgctagggtcgc
cctatcgctcggaacctggtcagctggctcccgcctcctgctcagcctcttccggtaccctt
tcttgcgctatgacacttccagcaaaaggtagggcgggctgcgagacggcttcccggcgctg
catgcaacaccgatgatgcttcgaccccccgaagctccttcggggctgcatgggcgctccga
tgccgctccagggcgagcgctgtttaaatagccaggcccccgattgcaaagacattatagcg
agctaccaaagccatattcaaacacctagatcactaccacttctacacaggccactcgagct
tgtgatcgcactccgctaagggggcgcctcttcctcttcgtttcagtcacaacccgcaaact
ctagaatatcaatgctgctgcaggccttcctgttcctgctggccggcttcgccgccaagatc
agcgcctccatgacgaacgagacgtccgaccgccccctggtgcacttcacccccaacaaggg
ctggatgaacgaccccaacggcctgtggtacgacgagaaggacgccaagtggcacctgtact
tccagtacaacccgaacgacaccgtctgggggacgcccttgttctggggccacgccacgtcc
gacgacctgaccaactgggaggaccagcccatcgccatcgccccgaagcgcaacgactccgg
cgccttctccggctccatggtggtggactacaacaacacctccggcttcttcaacgacacca
tcgacccgcgccagcgctgcgtggccatctggacctacaacaccccggagtccgaggagcag
tacatctcctacagcctggacggcggctacaccttcaccgagtaccagaagaaccccgtgct
ggccgccaactccacccagttccgcgacccgaaggtcttctggtacgagccctcccagaagt
ggatcatgaccgcggccaagtcccaggactacaagatcgagatctactcctccgacgacctg
aagtcctggaagctggagtccgcgttcgccaacgagggcttcctcggctaccagtacgagtg
ccccggcctgatcgaggtccccaccgagcaggaccccagcaagtcctactgggtgatgttca
tctccatcaaccccggcgccccggccggcggctccttcaaccagtacttcgtcggcagcttc
aacggcacccacttcgaggccttcgacaaccagtcccgcgtggtggacttcggcaaggacta
ctacgccctgcagaccttcttcaacaccgacccgacctacgggagcgccctgggcatcgcgt
gggcctccaactgggagtactccgccttcgtgcccaccaacccctggcgctcctccatgtcc
ctcgtgcgcaagttctccctcaacaccgagtaccaggccaacccggagacggagctgatcaa
cctgaaggccgagccgatcctgaacatcagcaacgccggcccctggagccggttcgccacca
acaccacgttgacgaaggccaacagctacaacgtcgacctgtccaacagcaccggcaccctg
gagttcgagctggtgtacgccgtcaacaccacccagacgatctccaagtccgtgttcgcgga
cctctccctctggttcaagggcctggaggaccccgaggagtacctccgcatgggcttcgagg
tgtccgcgtcctccttcttcctggaccgcgggaacagcaaggtgaagttcgtgaaggagaac
ccctacttcaccaaccgcatgagcgtgaacaaccagcccttcaagagcgagaacgacctgtc
ctactacaaggtgtacggcttgctggaccagaacatcctggagctgtacttcaacgacggcg
acgtcgtgtccaccaacacctacttcatgaccaccgggaacgccctgggctccgtgaacatg
acgacgggggtggacaacctgttctacatcgacaagttccaggtgcgcgaggtcaagtgaca
attgacgcccgcgcggcgcacctgacctgttctctcgagggcgcctgttctgccttgcgaaa
caagcccctggagcatgcgtgcatgatcgtctctggcgccccgccgcgcggtttgtcgccct
cgcgggcgccgcggccgcgggggcgcattgaaattgttgcaaaccccacctgacagattgag
ggcccaggcaggaaggcgttgagatggaggtacaggagtcaagtaactgaaagtttttatga
taactaacaacaaagggtcgtttctggccagcgaatgacaagaacaagattccacatttccg
tgtagaggcttgccatcgaatgtgagcgggcgggccgcggacccgacaaaacccttacgacg
tggtaagaaaaacgtggcgggcactgtccctgtagcctgaagaccagcaggagacgatcgga
agcatcacagcacaggatcccgcgtctcgaacagagcgcgcagaggaacgctgaaggtctcg
cctctgtcgcacctcagcgcggcatacaccacaataaccacctgacgaatgcgcttggttct
tcgtccattagcgaagcgtccggttcacacacgtgccacgttggcgaggtggcaggtgacaa
tgatcggtggagctgatggtcgaaacgttcacagcctagggaattcctgaagaatgggaggc
aggtgttgttgattatgagtgtgtaaaagaaaggggtagagagccgtcctcagatccgacta
ctatgcaggtagccgctcgcccatgcccgcctggctgaatattgatgcatgcccatcaaggc
aggcaggcatttctgtgcacgcaccaagcccacaatcttccacaacacacagcatgtaccaa
cgcacgcgtaaaagttggggtgctgccagtgcgtcatgccaggcatgatgtgctcctgcaca
tccgccatgatctcctccatcgtctcgggtgtttccggcgcctggtccgggagccgttccgc
cagatacccagacgccacctccgacctcacggggtacttttcgagcgtctgccggtagtcga
cgatcgcgtccaccatggagtagccgaggcgccggaactggcgtgacggagggaggagaggg
aggagagagaggggggggggggggggggatgattacacgccagtctcacaacgcatgcaaga
cccgtttgattatgagtacaatcatgcactactagatggatgagcgccaggcataaggcaca
ccgacgttgatggcatgagcaactcccgcatcatatttcctattgtcctcacgccaagccgg
tcaccatccgcatgctcatattacagcgcacgcaccgcttcgtgatccaccgggtgaacgta
gtcctcgacggaaacatctggctcgggcctcgtgctggcactccctcccatgccgacaacct
ttctgctgtcaccacgacccacgatgcaacgcgacacgacccggtgggactgatcggttcac
tgcacctgcatgcaattgtcacaagcgcatactccaatcgtatccgtttgatttctgtgaaa
actcgctcgaccgcccgcgtcccgcaggcagcgatgacgtgtgcgtgacctgggtgtttcgt
cgaaaggccagcaaccccaaatcgcaggcgatccggagattgggatctgatccgagcttgga
ccagatcccccacgatgcggcacgggaactgcatcgactcggcgcggaacccagctttcgta
aatgccagattggtgtccgataccttgatttgccatcagcgaaacaagacttcagcagcgag
cgtatttggcgggcgtgctaccagggttgcatacattgcccatttctgtctggaccgcttta
ccggcgcagagggtgagttgatggggttggcaggcatcgaaacgcgcgtgcatggtgtgtgt
gtctgttttcggctgcacaatttcaatagtcggatgggcgacggtagaattgggtgttgcgc
tcgcgtgcatgcctcgccccgtcgggtgtcatgaccgggactggaatcccccctcgcgaccc
tcctgctaacgctcccgactctcccgcccgcgcgcaggatagactctagttcaaccaatcga
cacatatggcttccgcggcattcaccatgtcggcgtgccccgcgatgactggcagggcccct
ggggcacgtcgctccggacggccagtcgccacccgcctgaggtacgtattccagtgcctggt
ggccagctgcatcgacccctgcgaccagtaccgcagcagcgccagcctgagcttcctgggcg
acaacggcttcgccagcctgttcggcagcaagcccttcatgagcaaccgcggccaccgccgc
ctgcgccgcgccagccacagcggcgaggccatggccgtggccctgcagcccgcccaggaggc
cggcaccaagaagaagcccgtgatcaagcagcgccgcgtggtggtgaccggcatgggcgtgg
tgacccccctgggccacgagcccgacgtgttctacaacaacctgctggacggcgtgagcggc
atcagcgagatcgagaccttcgactgcacccagttccccacccgcatcgccggcgagatcaa
gagcttcagcaccgacggctgggtggcccccaagctgagcaagcgcatggacaagttcatgc
tgtacctgctgaccgccggcaagaaggccctggccgacggcggcatcaccgacgaggtgatg
aaggagctggacaagcgcaagtgcggcgtgctgatcggcagcggcatgggcggcatgaaggt
gttcaacgacgccatcgaggccctgcgcgtgagctacaagaagatgaaccccttctgcgtgc
ccttcgccaccaccaacatgggcagcgccatgctggccatggacctgggctggatgggcccc
aactacagcatcagcaccgcctgcgccaccagcaacttctgcatcctgaacgccgccaacca
catcatccgcggcgaggccgacatgatgctgtgcggcggcagcgacgccgtgatcatcccca
tcggcctgggcggcttcgtggcctgccgcgccctgagccagcgcaacagcgaccccaccaag
gccagccgcccctgggacagcaaccgcgacggcttcgtgatgggcgagggcgccggcgtgct
gctgctggaggagctggagcacgccaagaagcgcggcgccaccatctacgccgagttcctgg
gcggcagcttcacctgcgacgcctaccacatgaccgagccccaccccgagggcgccggcgtg
atcctgtgcatcgagaaggccctggcccaggccggcgtgagcaaggaggacgtgaactacat
caacgcccacgccaccagcaccagcgccggcgacatcaaggagtaccaggccctggcccgct
gcttcggccagaacagcgagctgcgcgtgaacagcaccaagagcatgatcggccacctgctg
ggcgccgccggcggcgtggaggccgtgaccgtggtgcaggccatccgcaccggctggattca
ccccaacctgaacctggaggaccccgacaaggccgtggacgccaagctgctggtgggcccca
agaaggagcgcctgaacgtgaaggtgggcctgagcaacagcttcggcttcggcggccacaac
agcagcatcctgttcgccccctgcaacgtgtgactcgaggcagcagcagctcggatagtatc
gacacactctggacgctggtcgtgtgatggactgttgccgccacacttgctgccttgacctg
tgaatatccctgccgcttttatcaaacagcctcagtgtgtttgatcttgtgtgtacgcgctt
ttgcgagttgctagctgcttgtgctatttgcgaataccacccccagcatccccttccctcgt
ttcatatcgcttgcatcccaaccgcaacttatctacgctgtcctgctatccctcagcgctgc
tcctgctcctgctcactgcccctcgcacagccttggtttgggctccgcctgtattctcctgg
tactgcaacctgtaaaccagcactgcaatgctgatgcacgggaagtagtgggatgggaacac
aaatggaaagcttctgaagaatgggaggcaggtgttgttgattatgagtgtgtaaaagaaag
gggtagagagccgtcctcagatccgactactatgcaggtagccgctcgcccatgcccgcctg
gctgaatattgatgcatgcccatcaaggcaggcaggcatttctgtgcacgcaccaagcccac
aatcttccacaacacacagcatgtaccaacgcacgcgtaaaagttggggtgctgccagtgcg
tcatgccaggcatgatgtgctcctgcacatccgccatgatctcctccatcgtctcgggtgtt
tccggcgcctggtccgggagccgttccgccagatacccagacgccacctccgacctcacggg
gtacttttcgagcgtctgccggtagtcgacgatcgcgtccaccatggagtagccgaggcgcc
ggaactggcgtgacggagggaggagagggaggagagagaggggggggggggggggggatgat
tacacgccagtctcacaacgcatgcaagacccgtttgattatgagtacaatcatgcactact
agatggatgagcgccaggcataaggcacaccgacgttgatggcatgagcaactcccgcatca
tatttcctattgtcctcacgccaagccggtcaccatccgcatgctcatattacagcgcacgc
accgcttcgtgatccaccgggtgaacgtagtcctcgacggaaacatctggctcgggcctcgt
gctggcactccctcccatgccgacaacctttctgctgtcaccacgacccacgatgcaacgcg
acacgacccggtgggactgatcggttcactgcacctgcatgcaattgtcacaagcgcatact
ccaatcgtatccgtttgatttctgtgaaaactcgctcgaccgcccgcgtcccgcaggcagcg
atgacgtgtgcgtgacctgggtgtttcgtcgaaaggccagcaaccccaaatcgcaggcgatc
cggagattgggatctgatccgagcttggaccagatcccccacgatgcggcacgggaactgca
tcgactcggcgcggaacccagctttcgtaaatgccagattggtgtccgataccttgatttgc
catcagcgaaacaagacttcagcagcgagcgtatttggcgggcgtgctaccagggttgcata
cattgcccatttctgtctggaccgctttaccggcgcagagggtgagttgatggggttggcag
gcatcgaaacgcgcgtgcatggtgtgtgtgtctgttttcggctgcacaatttcaatagtcgg
atgggcgacggtagaattgggtgttgcgctcgcgtgcatgcctcgccccgtcgggtgtcatg
accgggactggaatcccccctcgcgaccctcctgctaacgctcccgactctcccgcccgcgc
gcaggatagactctagttcaaccaatcgacaactagtatggccaccgcctccaccttctccg
ccttcaacgcccgctgcggcgacctgcgccgctccgccggctccggcccccgccgccccgcc
cgccccctgcccgtgcgcgccgccatcaacgcctccgcccaccccaaggccaacggctccgc
cgtgaacctgaagtccggctccctgaacacccaggaggacacctcctcctcccccccccccc
gcgccttcctgaaccagctgcccgactggtccatgctgctgaccgccatcaccaccgtgttc
gtggccgccgagaagcagtggaccatgcgcgaccgcaagtccaagcgccccgacatgctggt
ggactccgtgggcctgaagtccgtggtgctggacggcctggtgtcccgccagatcttctcca
tccgctcctacgagatcggcgccgaccgcaccgcctccatcgagaccctgatgaaccacctg
caggagacctccatcaaccactgcaagtccctgggcctgctgaacgacggcttcggccgcac
ccccggcatgtgcaagaacgacctgatctgggtgctgaccaagatgcagatcatggtgaacc
gctaccccacctggggcgacaccgtggagatcaacacctggttctcccactccggcaagatc
ggcatggcctccgactggctgatcaccgactgcaacaccggcgagatcctgatccgcgccac
ctccgtgtgggccatgatgaaccagaagacccgccgcttctcccgcctgccctacgaggtgc
gccaggagctgaccccccactacgtggactccccccacgtgatcgaggacaacgaccgcaag
ctgcacaagttcgacgtgaagaccggcgactccatccgcaagggcctgaccccccgctggaa
cgacctggacgtgaaccagcacgtgtccaacgtgaagtacatcggctggatcctggagtcca
tgcccatcgaggtgctggagacccaggagctgtgctccctgaccgtggagtaccgccgcgag
tgcggcatggactccgtgctggagtccgtgaccgccatggacccctccgaggacgagggccg
ctcccagtacaagcacctgctgcgcctggaggacggcaccgacatcgtgaagggccgcaccg
agtggcgccccaagaacgccggcaccaacggcgccatctccaccgccaagccctccaacggc
aactccgtgtccatggactacaaggaccacgacggcgactacaaggaccacgacatcgacta
caaggacgacgacgacaagtgactcgaggcagcagcagctcggatagtatcgacacactctg
gacgctggtcgtgtgatggactgttgccgccacacttgctgccttgacctgtgaatatccct
gccgcttttatcaaacagcctcagtgtgtttgatcttgtgtgtacgcgcttttgcgagttgc
tagctgcttgtgctatttgcgaataccacccccagcatccccttccctcgtttcatatcgct
tgcatcccaaccgcaacttatctacgctgtcctgctatccctcagcgctgctcctgctcctg
ctcactgcccctcgcacagccttggtttgggctccgcctgtattctcctggtactgcaacct
gtaaaccagcactgcaatgctgatgcacgggaagtagtgggatgggaacacaaatggaaagc
tgtatagggataacagggtaatgagctcagcgtctgcgtgttgggagctggagtcgtgggct
tgacgacggcgctgcagctgttgcaggatgtgcctggcgtgcgcgttcacgtcgtggctgag
aaatatggcgacgaaacgttgacggctggggccggcgggctgtggatgccatacgcattggg
tacgcggccattggatgggattgataggcttatggagggataatagagtttttgccggatcc
aacgcatgtggatgcggtatcccggtgggctgaaagtgtggaaggatagtgcattggctatt
cacatgcactgcccaccccttttggcaggaaatgtgccggcatcgttggtgcaccgatgggg
aaaatcgacgttcgaccactacatgaagatttatacgtctgaagatgcagcgactgcgggtg
cgaaacggatgacggtttggtcgtgtatgtcacagcatgtgctggatcttgcgggctaactc
cccctgccacggcccattgcaggtgtcatgttgactggagggtacgacctttcgtccgtcaa
attcccagaggaggacccgctctgggccgacattgtgcccact
DAO1b-5′-nucleotides 1-735
CrTUB2-nucleotides 742-1053
ScSUC2-nucleotides 1066-2664
PmPGH 3′UTR-nucleotides 2671-3114
PmSAD2-2p-nucleotides 3333-4776
PmSADtp-CwKASAI-nucleotides 4780-6357
CvNR-nucleotides 6364-6764
PmSAD2-2p-nucleotides 6772-8215
CpSAD1tp_trimmed:CpauFATB1-nucleotides 8222-9508
CvNR-nucleotides 9515-9916
DAO1b-3′-nucleotides 9949-10521
SEQ ID NO: 16
D3798/pSZ4902 Sequence
Construct D3798 is written as KASI-2ver2_5′::PmHXT1-2v2-
ScarMEL1-PmPGK:CvNR:PmSAD2-2v3-PmSADtp-CpauKASIVa-CvNR:PmSAD2-
2v3-CpSAD1tp_tr2-CcFATB4-CvNR::KAS1-2ver2_3′
gtctaggttgcgaggtgactggccaggaagcagcaggcttggggtttggtgttctgatttct
ggtaatttgaggtttcattataagattctgtacggtcttgtttcgaaaacatgcaacaactc
cacacacacacactcctctcaactgagtctgcaggtttgacatctccgagttcccgaccaag
tttgcggcgcagatcaccggcttctccgtggaggactgcgtggacaagaagaacgcgcggcg
gtacgacgacgcgctgtcgtacgcgatggtggcctccaagaaggccctgcgccaggcaggcc
tggagaaggacaagtgccccgagggctacggggcgctggacaagacgcgcacgggcgtgctg
gtcggctcgggcatgggcgggctgacggtcttccaggacggcgtcaaggcgctggtggagaa
gggctacaagaagatgagccccttcttcatcccctacgccatcaccaacatgggctccgcgc
tggtgggcatcgaccagggcttcatgggccccaactactccgtctccacagcctgcgcgacg
tccaactacgcatttgtgaacgcggccaaccacatccgcaagggcgacgcggacgtcatggt
cgtcggcggcaccgaggcctccatcgtgcccgtgggcctgggcggctttgtggcctgccgcg
cgctgtccacgcgcaacgacgagcccaagcgcgcgagccggccgtgggacgagggccgcgac
ggctttggtaccccgctcccgtctggtcctcacgttcgtgtacggcctggatcccggaaagg
gcggatgcacgtggtgttgccccgccattggcgcccacgtttcaaagtccccggccagaaat
gcacaggaccggcccggctcgcacaggccatgacgaatgcccagatttcgacagcaaaacaa
tctggaataatcgcaaccattcgcgttttgaacgaaacgaaaagacgctgtttagcacgttt
ccgatatcgtgggggccgaagcatgattggggggaggaaagcgtggccccaaggtagcccat
tctgtgccacacgccgacgaggaccaatccccggcatcagccttcatcgacggctgcgccgc
acatataaagccggacgccttcccgacacgttcaaacagttttatttcctccacttcctgaa
tcaaacaaatcttcaaggaagatcctgctcttgagcaactagtatgttcgcgttctacttcc
tgacggcctgcatctccctgaagggcgtgttcggcgtctccccctcctacaacggcctgggc
ctgacgccccagatgggctgggacaactggaacacgttcgcctgcgacgtctccgagcagct
gctgctggacacggccgaccgcatctccgacctgggcctgaaggacatgggctacaagtaca
tcatcctggacgactgctggtcctccggccgcgactccgacggcttcctggtcgccgacgag
cagaagttccccaacggcatgggccacgtcgccgaccacctgcacaacaactccttcctgtt
cggcatgtactcctccgcgggcgagtacacgtgcgccggctaccccggctccctgggccgcg
aggaggaggacgcccagttcttcgcgaacaaccgcgtggactacctgaagtacgacaactgc
tacaacaagggccagttcggcacgcccgagatctcctaccaccgctacaaggccatgtccga
cgccctgaacaagacgggccgccccatcttctactccctgtgcaactggggccaggacctga
ccttctactggggctccggcatcgcgaactcctggcgcatgtccggcgacgtcacggcggag
ttcacgcgccccgactcccgctgcccctgcgacggcgacgagtacgactgcaagtacgccgg
cttccactgctccatcatgaacatcctgaacaaggccgcccccatgggccagaacgcgggcg
tcggcggctggaacgacctggacaacctggaggtcggcgtcggcaacctgacggacgacgag
gagaaggcgcacttctccatgtgggccatggtgaagtcccccctgatcatcggcgcgaacgt
gaacaacctgaaggcctcctcctactccatctactcccaggcgtccgtcatcgccatcaacc
aggactccaacggcatccccgccacgcgcgtctggcgctactacgtgtccgacacggacgag
tacggccagggcgagatccagatgtggtccggccccctggacaacggcgaccaggtcgtggc
gctgctgaacggcggctccgtgtcccgccccatgaacacgaccctggaggagatcttcttcg
actccaacctgggctccaagaagctgacctccacctgggacatctacgacctgtgggcgaac
cgcgtcgacaactccacggcgtccgccatcctgggccgcaacaagaccgccaccggcatcct
gtacaacgccaccgagcagtcctacaaggacggcctgtccaagaacgacacccgcctgttcg
gccagaagatcggctccctgtcccccaacgcgatcctgaacacgaccgtccccgcccacggc
atcgcgttctaccgcctgcgcccctcctcctgatacaacttattacgtattctgaccggcgc
tgatgtggcgcggacgccgtcgtactctttcagactttactcttgaggaattgaacctttct
cgcttgctggcatgtaaacattggcgcaattaattgtgtgatgaagaaagggtggcacaaga
tggatcgcgaatgtacgagatcgacaacgatggtgattgttatgaggggccaaacctggctc
aatcttgtcgcatgtccggcgcaatgtgatccagcggcgtgactctcgcaacctggtagtgt
gtgcgcaccgggtcgctttgattaaaactgatcgcattgccatcccgtcaactcacaagcct
actctagctcccattgcgcactcgggcgcccggctcgatcaatgttctgagcggagggcgaa
gcgtcaggaaatcgtctcggcagctggaagcgcatggaatgcggagcggagatcgaatcagg
atccgcagcagcagctcggatagtatcgacacactctggacgctggtcgtgtgatggactgt
tgccgccacacttgctgccttgacctgtgaatatccctgccgcttttatcaaacagcctcag
tgtgtttgatcttgtgtgtacgcgcttttgcgagttgctagctgcttgtgctatttgcgaat
accacccccagcatccccttccctcgtttcatatcgcttgcatcccaaccgcaacttatcta
cgctgtcctgctatccctcagcgctgctcctgctcctgctcactgcccctcgcacagccttg
gtttgggctccgcctgtattctcctggtactgcaacctgtaaaccagcactgcaatgctgat
gcacgggaagtagtgggatgggaacacaaatggaaagctgtagaattcgtgaaaactctctc
gaccgcccgcgtcccgcaggcagcgatgacgtgtgcgtgacctgggtgtttcgtcgaaaggc
cagcaaccccaaatcgcaggcgatccggagattgggatctgatccgagcttggaccagatcc
cccacgatgcggcacgggaactgcatcgactcggcgcggaacccagctttcgtaaatgccag
attggtgtccgataccttgatttgccatcagcgaaacaagacttcagcagcgagcgtatttg
gcgggcgtgctaccagggttgcatacattgcccatttctgtctggaccgctttaccggcgca
gagggtgagttgatggggttggcaggcatcgaaacgcgcgtgcatggtgtgtgtgtctgttt
tcggctgcacaatttcaatagtcggatgggcgacggtagaattgggtgttgcgctcgcgtgc
atgcctcgccccgtcgggtgtcatgaccgggactggaatcccccctcgcgaccctcctgcta
acgctcccgactctcccgcccgcgcgcaggatagactctagttcaaccaatcgacaactagt
aacaatggcttccgcggcattcaccatgtcggcgtgccccgcgatgactggcagggcccctg
gggcacgtcgctccggacggccagtcgccacccgcctgaggggctccaccttccagtgcctg
gtgaactcccacatcgacccctgcaaccagaacgtgtcctccgcctccctgtccttcctggg
cgacaacggcttcggctccaaccccttccgctccaaccgcggccaccgccgcctgggccgcg
cctcccactccggcgaggccatggccgtggccctgcagcccgcccaggaggtggccaccaag
aagaagcccgccatcaagcagcgccgcgtggtggtgaccggcatgggcgtggtgacccccct
gggccacgagcccgacgtgttctacaacaacctgctggacggcgtgtccggcatctccgaga
tcgagaccttcgactgcacccagttccccacccgcatcgccggcgagatcaagtccttctcc
accgacggctgggtggcccccaagctgtccaagcgcatggacaagttcatgctgtacctgct
gaccgccggcaagaaggccctggccgacgccggcatcaccgaggacgtgatgaaggagctgg
acaagcgcaagtgcggcgtgctgatcggctccggcatgggcggcatgaagctgttcaacgac
tccatcgaggccctgcgcgtgtcctacaagaagatgaaccccttctgcgtgcccttcgccac
caccaacatgggctccgccatgctggccatggacctgggctggatgggccccaactactcca
tctccaccgcctgcgccacctccaacttctgcatcctgaacgccgccaaccacatcatccgc
ggcgaggccgacatgatgctgtgcggcggctccgacgccgtgatcatccccatcggcctggg
cggcttcgtggcctgccgcgccctgtcccagcgcaactccgaccccaccaaggcctcccgcc
cctgggactccaaccgcgacggcttcgtgatgggcgagggcgccggcgtgctgctgctggag
gagctggagcacgccaagaagcgcggcgccaccatctacgccgagttcctgggcggctcctt
cacctgcgacgcctaccacatgaccgagccccaccccgacggcgccggcgtgatcctgtgca
tcgagaaggccctggcccagtccggcgtgtcccgcgaggacgtgaactacatcaacgcccac
gccacctccacccccgccggcgacatcaaggagtaccaggccctggcccactgcttcggcca
gaactccgagctgcgcgtgaactccaccaagtccatgatcggccacctgctgggcgccgccg
gcggcgtggaggccgtgaccgtgatccaggccatccgcaccggctggatccaccccaacctg
aacctggaggaccccgacgaggccgtggacgccaagttcctggtgggccccaagaaggagcg
cctgaacgtgaaggtgggcctgtccaactccttcggcttcggcggccacaactcctccatcc
tgttcgccccctacaacaccatgtacccctacgacgtgcccgactacgcctgatatcgaggc
agcagcagctcggatagtatcgacacactctggacgctggtcgtgtgatggactgttgccgc
cacacttgctgccttgacctgtgaatatccctgccgcttttatcaaacagcctcagtgtgtt
tgatcttgtgtgtacgcgcttttgcgagttgctagctgcttgtgctatttgcgaataccacc
cccagcatccccttccctcgtttcatatcgcttgcatcccaaccgcaacttatctacgctgt
cctgctatccctcagcgctgctcctgctcctgctcactgcccctcgcacagccttggtttgg
gctccgcctgtattctcctggtactgcaacctgtaaaccagcactgcaatgctgatgcacgg
gaagtagtgggatgggaacacaaatggaaagcttgagacggtgaaaactcgctcgaccgccc
gcgtcccgcaggcagcgatgacgtgtgcgtgacctgggtgtttcgtcgaaaggccagcaacc
ccaaatcgcaggcgatccggagattgggatctgatccgagcttggaccagatcccccacgat
gcggcacgggaactgcatcgactcggcgcggaacccagctttcgtaaatgccagattggtgt
ccgataccttgatttgccatcagcgaaacaagacttcagcagcgagcgtatttggcgggcgt
gctaccagggttgcatacattgcccatttctgtctggaccgctttaccggcgcagagggtga
gttgatggggttggcaggcatcgaaacgcgcgtgcatggtgtgtgtgtctgttttcggctgc
acaatttcaatagtcggatgggcgacggtagaattgggtgttgcgctcgcgtgcatgcctcg
ccccgtcgggtgtcatgaccgggactggaatcccccctcgcgaccctcctgctaacgctccc
gactctcccgcccgcgcgcaggatagactctagttcaaccaatcgacaactagtaacaatgg
ccaccgcctccaccttctccgccttcaacgcccgctgcggcgacctgcgccgctccgccggc
tccggcccccgccgccccgcccgccccctgcccgtgcgcgccgccatcggcaacgagcgcaa
ctcctgcaaggtgatcaacggcaccaaggtgaaggacaccgagggcctgaagggctgctcca
ccctgcagggccagtccatgctggacgaccacttcggcctgcacggcctggtgttccgccgc
accttcgccatccgctgctacgaggtgggccccgaccgctccacctccatcatggccgtgat
gaaccacctgcaggaggccgcccgcaaccacgccgagtccctgggcctgctgggcgacggct
tcggcgagaccctggagatgtccaagcgcgacctgatctgggtggtgcgccgcacccacgtg
gccgtggagcgctaccccgcctggggcgacaccgtggaggtggaggcctgggtgggcgcctc
cggcaacaccggcatgcgccgcgacttcctggtgcgcgactgcaagaccggccacatcctga
cccgctgcacctccgtgtccgtgatgatgaacatgcgcacccgccgcctgtccaagatcccc
caggaggtgcgcgccgagatcgaccccctgttcatcgagaaggtggccgtgaaggagggcga
gatcaagaagctgcagaagctgaacgactccaccgccgactacatccagggcggctggaccc
cccgctggaacgacctggacgtgaaccagcacgtgaacaacatcatctacgtgggctggatc
ttcaagtccgtgcccgactccatctccgagaaccaccacctgtcctccatcaccctggagta
ccgccgcgagtgcacccgcggcaacaagctgcagtccctgaccaccgtgtgcggcggctcct
ccgaggccggcatcatctgcgagcacctgctgcagctggaggacggctccgaggtgctgcgc
gcccgcaccgagtggcgccccaagcacaccgactccttccagggcatctccgagcgcttccc
ccagcaggagccccacaaggactacaaggaccacgacggcgactacaaggaccacgacatcg
actacaaggacgacgacgacaagtgactcgaggcagcagcagctcggatagtatcgacacac
tctggacgctggtcgtgtgatggactgttgccgccacacttgctgccttgacctgtgaatat
ccctgccgcttttatcaaacagcctcagtgtgtttgatcttgtgtgtacgcgcttttgcgag
ttgctagctgcttgtgctatttgcgaataccacccccagcatccccttccctcgtttcatat
cgcttgcatcccaaccgcaacttatctacgctgtcctgctatccctcagcgctgctcctgct
cctgctcactgcccctcgcacagccttggtttgggctccgcctgtattctcctggtactgca
acctgtaaaccagcactgcaatgctgatgcacgggaagtagtgggatgggaacacaaatgga
aagcttgagctcgtgatgggcgagggcgcggccgtgctggtcatggagtcgctggagcacgc
gcagaagcgtggcgcgaccatcctgggcgagtacctgggcggcgccatgacctgcgacgcgc
accacatgacggacccgcaccccgagggcctgggcgtgagcacctgcatccgcctggcgctc
gaggacgccggcgtctcgcccgacgaggtcaactacgtcaacgcgcacgccacctccaccct
ggtgggcgacaaggccgaggtgcgcgcggtcaagtcggtctttggcgacatgaagggtatca
agatgaacgccaccaagagtatgatcgggcactgcctgggcgccgccggcggcatggaggcc
gtcgcgacgctcatggccatccgcaccggctgggtgcaccccaccatcaaccacgacaaccc
catcgccgaggtcgatggcctggacgtcgtcgccaacgccaaggcccagcacgacatcaacg
tcgccatctccaactccttcggctttggcgggcacaactccgtcgtcgcctttgcgcccttc
cgcgagtaggtgaagcgagcgtgctttgctgaggagggaggcggggtgcgagcgctctggcc
gtgcgcgcgatactctccccgcatgagcagactcctcgtgccacgcccgaatctacttgtca
acgagcaactgtgtgttttgtccgtggccaatcttattatttctccgactgtggccgtactc
tgtttggctgtgcaagcacc
KSI-2ver2_5′::PmHXT1-2v2-ScarMEL1-PmPGK:CvNR:PmSAD2-2v3-
PmSADtp-CpauKASIVa-CvNR:PmSAD2-2v3-CpSAD1tp_tr2-CcFATB4-
CvNR::KAS1-2ver2_3′
KSI-2ver2_5′-nucleotides 1-750
PmHXTI-2v2-nucleotides 757-1215
ScarMEL1-nucleotides 1222-2637
PmPGK 3′UTR-nucleotides 2654-3098
CvNR-nucleotides 3105-3506
PmSAD2-2v3-nucleotides 3521-4086
PmSADtp-CpauKASIVa-nucleotides 4093-5695
CvNR-nucleotides 5703-6104
PmSAD2-2v3-nucleotides 6117-6682
CpSAD1tp_tr2-CcFATB4 - 6693-7838
CvNR-nucleotides 7845-8246
KAS1-2ver2_3′-nucleotides 8259-9010
SEQ ID NO: 17
D3104/pSZ4330 Sequence
Construct D3104 is written as THI4a::CrTUB2-ScSUC2-
PmPGH:PmACP1-1p-CpSAD1tp_ChFATB2ExtC_FLAG-CvNR::THI4a
ccctcaactgcgacgctgggaaccttctccgggcaggcgatgtgcgtgggtttgcctccttg
gcacggctctacaccgtcgagtacgccatgaggcggtgatggctgtgtcggttgccacttcg
tccagagacggcaagtcgtccatcctctgcgtgtgtggcgcgacgctgcagcagtccctctg
cagcagatgagcgtgactttggccatttcacgcactcgagtgtacacaatccatttttctta
aagcaaatgactgctgattgaccagatactgtaacgctgatttcgctccagatcgcacagat
agcgaccatgttgctgcgtctgaaaatctggattccgaattcgaccctggcgctccatccat
gcaacagatggcgacacttgttacaattcctgtcacccatcggcatggagcaggtccactta
gattcccgatcacccacgcacatctcgctaatagtcattcgttcgtgtcttcgatcaatctc
aagtgagtgtgcatggatcttggttgacgatgcggtatgggtttgcgccgctggctgcaggg
tctgcccaaggcaagctaacccagctcctctccccgacaatactctcgcaggcaaagccggt
cacttgccttccagattgccaataaactcaattatggcctctgtcatgccatccatgggtct
gatgaatggtcacgctcgtgtcctgaccgttccccagcctctggcgtcccctgccccgccca
ccagcccacgccgcgcggcagtcgctgccaaggctgtctcggaggtaccctttcttgcgcta
tgacacttccagcaaaaggtagggcgggctgcgagacggcttcccggcgctgcatgcaacac
cgatgatgcttcgaccccccgaagctccttcggggctgcatgggcgctccgatgccgctcca
gggcgagcgctgtttaaatagccaggcccccgattgcaaagacattatagcgagctaccaaa
gccatattcaaacacctagatcactaccacttctacacaggccactcgagcttgtgatcgca
ctccgctaagggggcgcctcttcctcttcgtttcagtcacaacccgcaaactctagaatatc
aatgctgctgcaggccttcctgttcctgctggccggcttcgccgccaagatcagcgcctcca
tgacgaacgagacgtccgaccgccccctggtgcacttcacccccaacaagggctggatgaac
gaccccaacggcctgtggtacgacgagaaggacgccaagtggcacctgtacttccagtacaa
cccgaacgacaccgtctgggggacgcccttgttctggggccacgccacgtccgacgacctga
ccaactgggaggaccagcccatcgccatcgccccgaagcgcaacgactccggcgccttctcc
ggctccatggtggtggactacaacaacacctccggcttcttcaacgacaccatcgacccgcg
ccagcgctgcgtggccatctggacctacaacaccccggagtccgaggagcagtacatctcct
acagcctggacggcggctacaccttcaccgagtaccagaagaaccccgtgctggccgccaac
tccacccagttccgcgacccgaaggtcttctggtacgagccctcccagaagtggatcatgac
cgcggccaagtcccaggactacaagatcgagatctactcctccgacgacctgaagtcctgga
agctggagtccgcgttcgccaacgagggcttcctcggctaccagtacgagtgccccggcctg
atcgaggtccccaccgagcaggaccccagcaagtcctactgggtgatgttcatctccatcaa
ccccggcgccccggccggcggctccttcaaccagtacttcgtcggcagcttcaacggcaccc
acttcgaggccttcgacaaccagtcccgcgtggtggacttcggcaaggactactacgccctg
cagaccttcttcaacaccgacccgacctacgggagcgccctgggcatcgcgtgggcctccaa
ctgggagtactccgccttcgtgcccaccaacccctggcgctcctccatgtccctcgtgcgca
agttctccctcaacaccgagtaccaggccaacccggagacggagctgatcaacctgaaggcc
gagccgatcctgaacatcagcaacgccggcccctggagccggttcgccaccaacaccacgtt
gacgaaggccaacagctacaacgtcgacctgtccaacagcaccggcaccctggagttcgagc
tggtgtacgccgtcaacaccacccagacgatctccaagtccgtgttcgcggacctctccctc
tggttcaagggcctggaggaccccgaggagtacctccgcatgggcttcgaggtgtccgcgtc
ctccttcttcctggaccgcgggaacagcaaggtgaagttcgtgaaggagaacccctacttca
ccaaccgcatgagcgtgaacaaccagcccttcaagagcgagaacgacctgtcctactacaag
gtgtacggcttgctggaccagaacatcctggagctgtacttcaacgacggcgacgtcgtgtc
caccaacacctacttcatgaccaccgggaacgccctgggctccgtgaacatgacgacggggg
tggacaacctgttctacatcgacaagttccaggtgcgcgaggtcaagtgacaattgacgccc
gcgcggcgcacctgacctgttctctcgagggcgcctgttctgccttgcgaaacaagcccctg
gagcatgcgtgcatgatcgtctctggcgccccgccgcgcggtttgtcgccctcgcgggcgcc
gcggccgcgggggcgcattgaaattgttgcaaaccccacctgacagattgagggcccaggca
ggaaggcgttgagatggaggtacaggagtcaagtaactgaaagtttttatgataactaacaa
caaagggtcgtttctggccagcgaatgacaagaacaagattccacatttccgtgtagaggct
tgccatcgaatgtgagcgggcgggccgcggacccgacaaaacccttacgacgtggtaagaaa
aacgtggcgggcactgtccctgtagcctgaagaccagcaggagacgatcggaagcatcacag
cacaggatcccgcgtctcgaacagagcgcgcagaggaacgctgaaggtctcgcctctgtcgc
acctcagcgcggcatacaccacaataaccacctgacgaatgcgcttggttcttcgtccatta
gcgaagcgtccggttcacacacgtgccacgttggcgaggtggcaggtgacaatgatcggtgg
agctgatggtcgaaacgttcacagcctagggatatcgcctgctcaagcgggcgctcaacatg
cagagcgtcagcgagacgggctgtggcgatcgcgagacggacgaggccgcctctgccctgtt
tgaactgagcgtcagcgctggctaaggggagggagactcatccccaggctcgcgccagggct
ctgatcccgtctcgggcggtgatcggcgcgcatgactacgacccaacgacgtacgagactga
tgtcggtcccgacgaggagcgccgcgaggcactcccgggccaccgaccatgtttacaccgac
cgaaagcactcgctcgtatccattccgtgcgcccgcacatgcatcatcttttggtaccgact
tcggtcttgttttacccctacgacctgccttccaaggtgtgagcaactcgcccggacatgac
cgagggtgatcatccggatccccaggccccagcagcccctgccagaatggctcgcgctttcc
agcctgcaggcccgtctcccaggtcgacgcaacctacatgaccaccccaatctgtcccagac
cccaaacaccctccttccctgcttctctgtgatcgctgatcagcaacaactagtaacaatgg
ccaccgcatccactttctcggcgttcaatgcccgctgcggcgacctgcgtcgctcggcgggc
tccgggccccggcgcccagcgaggcccctccccgtgcgcgggcgcgcctccagcctgagccc
ctccttcaagcccaagtccatccccaacggcggcttccaggtgaaggccaacgacagcgccc
accccaaggccaacggctccgccgtgagcctgaagagcggcagcctgaacacccaggaggac
acctcctccagcccccccccccgcaccttcctgcaccagctgcccgactggagccgcctgct
gaccgccatcaccaccgtgttcgtgaagtccaagcgccccgacatgcacgaccgcaagtcca
agcgccccgacatgctggtggacagcttcggcctggagtccaccgtgcaggacggcctggtg
ttccgccagtccttctccatccgctcctacgagatcggcaccgaccgcaccgccagcatcga
gaccctgatgaaccacctgcaggagacctccctgaaccactgcaagagcaccggcatcctgc
tggacggcttcggccgcaccctggagatgtgcaagcgcgacctgatctgggtggtgatcaag
atgcagatcaaggtgaaccgctaccccgcctggggcgacaccgtggagatcaacacccgctt
cagccgcctgggcaagatcggcatgggccgcgactggctgatctccgactgcaacaccggcg
agatcctggtgcgcgccaccagcgcctacgccatgatgaaccagaagacccgccgcctgtcc
aagctgccctacgaggtgcaccaggagatcgtgcccctgttcgtggacagccccgtgatcga
ggactccgacctgaaggtgcacaagttcaaggtgaagaccggcgacagcatccagaagggcc
tgacccccggctggaacgacctggacgtgaaccagcacgtgtccaacgtgaagtacatcggc
tggatcctggagagcatgcccaccgaggtgctggagacccaggagctgtgctccctggccct
ggagtaccgccgcgagtgcggccgcgactccgtgctggagagcgtgaccgccatggacccca
gcaaggtgggcgtgcgctcccagtaccagcacctgctgcgcctggaggacggcaccgccatc
gtgaacggcgccaccgagtggcgccccaagaacgccggcgccaacggcgccatctccaccgg
caagaccagcaacggcaactccgtgtccatggactacaaggaccacgacggcgactacaagg
accacgacatcgactacaaggacgacgacgacaagtgactcgaggcagcagcagctcggata
gtatcgacacactctggacgctggtcgtgtgatggactgttgccgccacacttgctgccttg
acctgtgaatatccctgccgcttttatcaaacagcctcagtgtgtttgatcttgtgtgtacg
cgcttttgcgagttgctagctgcttgtgctatttgcgaataccacccccagcatccccttcc
ctcgtttcatatcgcttgcatcccaaccgcaacttatttacgctgtcctgctatccctcagc
gctgctcctgctcctgctcactgcccctcgcacagccttggtttgggctccgcctgtattct
cctggtactgcaacctgtaaaccagcactgcaatgctgatgcacgggaagtagtgggatggg
aacacaaatggaaagctgtatagggataacagggtaatgagctccagcgccatgccacgccc
tttgatggcttcaagtacgattacggtgttggattgtgtgtttgttgcgtagtgtgcatggt
ttagaataatacacttgatttcttgctcacggcaatctcggcttgtccgcaggttcaacccc
atttcggagtctcaggtcagccgcgcaatgaccagccgctacttcaaggacttgcacgacaa
cgccgaggtgagctatgtttaggacttgattggaaattgtcgtcgacgcatattcgcgctcc
gcgacagcacccaagcaaaatgtcaagtgcgttccgatttgcgtccgcaggtcgatgttgtg
atcgtcggcgccggatccgccggtctgtcctgcgcttacgagctgaccaagcaccctgacgt
ccgggtacgcgagctgagattcgattagacataaattgaagattaaacccgtagaaaaattt
gatggtcgcgaaactgtgctcgattgcaagaaattgatcgtcctccactccgcaggtcgcca
tcatcgagcagggcgttgctcccggcggcggcgcctggctggggggacagctgttctcggcc
atgtgtgtacgtagaaggatgaatttcagctggttttcgttgcacagctgtttgtgcatgat
ttgtttcagactattgttgaatgtttttagatttcttaggatgcatgatttgtctgcatgcg
act
THI4a::CrTUB2-ScSUC2-PmPGH:PmACP1-1p-
CpSAD1tp_ChFATB2ExtC_FLAG-CvNR::THI4a
THI4A_5′-nucleotides 1-787
CrTUB2-nucletodies 794-1105
ScSUC2-nucleotides 1118-2716
PmPGH-nucleotides 2723-3166
PmACP1-1p-nucleotides 3385-3955
CpSAD1tp_ChFATB2ExtC_FLAG-nucleotides 3965-5308
CvNR-nucleotides 5315-5716
THI4A_3′-nucleotides 5749-6451
SEQ ID NO: 18
D3937/pSZ5075 Sequence
Construct D3937 is written as KASI-1ver2_5′::PmHXT1-2v2-
ScarMEL1-PmPGK:CvNR:PmSAD2-2v3-PmSADtp-CpauKASIVa-CvNR:PmACP1-
1p-CpSAD1tp_trmd:CcFATB4-CvNR::KAS1-1ver2_3′
gtctaggttgggaggcggctggcgaggaagcagcaggcttggggtttggtgttccgatttct
ggcaatttgaggtttcattgtgagattctatgcggtcttgtttcgaaaacatgcaacaactc
cacacacacacactcctctccaccaactctgcaggtttgacatctccgagttcccgaccaag
tttgcggcgcagatcaccggcttctccgtggaggactgcgtggacaagaagaacgcgcggcg
gtacgacgacgcgctgtcgtacgcgatggtggcctccaagaaggccctgcgccaggcgggac
tggagaaggacaagtgccccgagggctacggagcgctggataagacgcgcgcgggcgtgctg
gtcggctcgggcatgggcgggctgacggtcttccaggacggcgtcaaggcgctggtggagaa
gggctacaagaagatgagccccttcttcatcccctacgccatcaccaacatgggctccgcgc
tggtgggcatcgaccagggcttcatggggcccaactactccgtctccacggcctgcgcgacc
tccaactacgcctttgtgaacgcggccaaccacatccgcaagggcgacgcggacgtcatggt
cgtgggcggcaccgaggcctccatcgtgcccgtgggcctgggcggctttgtggcctgccgcg
cgctgtccacgcgcaacgacgagcccaagcgcgcgagccggccgtgggacgagggccgcgac
ggcttcggtaccccgctcccgtctggtcctcacgttcgtgtacggcctggatcccggaaagg
gcggatgcacgtggtgttgccccgccattggcgcccacgtttcaaagtccccggccagaaat
gcacaggaccggcccggctcgcacaggccatgacgaatgcccagatttcgacagcaaaacaa
tctggaataatcgcaaccattcgcgttttgaacgaaacgaaaagacgctgtttagcacgttt
ccgatatcgtgggggccgaagcatgattggggggaggaaagcgtggccccaaggtagcccat
tctgtgccacacgccgacgaggaccaatccccggcatcagccttcatcgacggctgcgccgc
acatataaagccggacgccttcccgacacgttcaaacagttttatttcctccacttcctgaa
tcaaacaaatcttcaaggaagatcctgctcttgagcaactagtatgttcgcgttctacttcc
tgacggcctgcatctccctgaagggcgtgttcggcgtctccccctcctacaacggcctgggc
ctgacgccccagatgggctgggacaactggaacacgttcgcctgcgacgtctccgagcagct
gctgctggacacggccgaccgcatctccgacctgggcctgaaggacatgggctacaagtaca
tcatcctggacgactgctggtcctccggccgcgactccgacggcttcctggtcgccgacgag
cagaagttccccaacggcatgggccacgtcgccgaccacctgcacaacaactccttcctgtt
cggcatgtactcctccgcgggcgagtacacgtgcgccggctaccccggctccctgggccgcg
aggaggaggacgcccagttcttcgcgaacaaccgcgtggactacctgaagtacgacaactgc
tacaacaagggccagttcggcacgcccgagatctcctaccaccgctacaaggccatgtccga
cgccctgaacaagacgggccgccccatcttctactccctgtgcaactggggccaggacctga
ccttctactggggctccggcatcgcgaactcctggcgcatgtccggcgacgtcacggcggag
ttcacgcgccccgactcccgctgcccctgcgacggcgacgagtacgactgcaagtacgccgg
cttccactgctccatcatgaacatcctgaacaaggccgcccccatgggccagaacgcgggcg
tcggcggctggaacgacctggacaacctggaggtcggcgtcggcaacctgacggacgacgag
gagaaggcgcacttctccatgtgggccatggtgaagtcccccctgatcatcggcgcgaacgt
gaacaacctgaaggcctcctcctactccatctactcccaggcgtccgtcatcgccatcaacc
aggactccaacggcatccccgccacgcgcgtctggcgctactacgtgtccgacacggacgag
tacggccagggcgagatccagatgtggtccggccccctggacaacggcgaccaggtcgtggc
gctgctgaacggcggctccgtgtcccgccccatgaacacgaccctggaggagatcttcttcg
actccaacctgggctccaagaagctgacctccacctgggacatctacgacctgtgggcgaac
cgcgtcgacaactccacggcgtccgccatcctgggccgcaacaagaccgccaccggcatcct
gtacaacgccaccgagcagtcctacaaggacggcctgtccaagaacgacacccgcctgttcg
gccagaagatcggctccctgtcccccaacgcgatcctgaacacgaccgtccccgcccacggc
atcgcgttctaccgcctgcgcccctcctcctgatacaacttattacgtattctgaccggcgc
tgatgtggcgcggacgccgtcgtactctttcagactttactcttgaggaattgaacctttct
cgcttgctggcatgtaaacattggcgcaattaattgtgtgatgaagaaagggtggcacaaga
tggatcgcgaatgtacgagatcgacaacgatggtgattgttatgaggggccaaacctggctc
aatcttgtcgcatgtccggcgcaatgtgatccagcggcgtgactctcgcaacctggtagtgt
gtgcgcaccgggtcgctttgattaaaactgatcgcattgccatcccgtcaactcacaagcct
actctagctcccattgcgcactcgggcgcccggctcgatcaatgttctgagcggagggcgaa
gcgtcaggaaatcgtctcggcagctggaagcgcatggaatgcggagcggagatcgaatcagg
atccgcagcagcagctcggatagtatcgacacactctggacgctggtcgtgtgatggactgt
tgccgccacacttgctgccttgacctgtgaatatccctgccgcttttatcaaacagcctcag
tgtgtttgatcttgtgtgtacgcgcttttgcgagttgctagctgcttgtgctatttgcgaat
accacccccagcatccccttccctcgtttcatatcgcttgcatcccaaccgcaacttatcta
cgctgtcctgctatccctcagcgctgctcctgctcctgctcactgcccctcgcacagccttg
gtttgggctccgcctgtattctcctggtactgcaacctgtaaaccagcactgcaatgctgat
gcacgggaagtagtgggatgggaacacaaatggaaagctgtagaattcgtgaaaactctctc
gaccgcccgcgtcccgcaggcagcgatgacgtgtgcgtgacctgggtgtttcgtcgaaaggc
cagcaaccccaaatcgcaggcgatccggagattgggatctgatccgagcttggaccagatcc
cccacgatgcggcacgggaactgcatcgactcggcgcggaacccagctttcgtaaatgccag
attggtgtccgataccttgatttgccatcagcgaaacaagacttcagcagcgagcgtatttg
gcgggcgtgctaccagggttgcatacattgcccatttctgtctggaccgctttaccggcgca
gagggtgagttgatggggttggcaggcatcgaaacgcgcgtgcatggtgtgtgtgtctgttt
tcggctgcacaatttcaatagtcggatgggcgacggtagaattgggtgttgcgctcgcgtgc
atgcctcgccccgtcgggtgtcatgaccgggactggaatcccccctcgcgaccctcctgcta
acgctcccgactctcccgcccgcgcgcaggatagactctagttcaaccaatcgacaactagt
aacaatggcttccgcggcattcaccatgtcggcgtgccccgcgatgactggcagggcccctg
gggcacgtcgctccggacggccagtcgccacccgcctgaggggctccaccttccagtgcctg
gtgaactcccacatcgacccctgcaaccagaacgtgtcctccgcctccctgtccttcctggg
cgacaacggcttcggctccaaccccttccgctccaaccgcggccaccgccgcctgggccgcg
cctcccactccggcgaggccatggccgtggccctgcagcccgcccaggaggtggccaccaag
aagaagcccgccatcaagcagcgccgcgtggtggtgaccggcatgggcgtggtgacccccct
gggccacgagcccgacgtgttctacaacaacctgctggacggcgtgtccggcatctccgaga
tcgagaccttcgactgcacccagttccccacccgcatcgccggcgagatcaagtccttctcc
accgacggctgggtggcccccaagctgtccaagcgcatggacaagttcatgctgtacctgct
gaccgccggcaagaaggccctggccgacgccggcatcaccgaggacgtgatgaaggagctgg
acaagcgcaagtgcggcgtgctgatcggctccggcatgggcggcatgaagctgttcaacgac
tccatcgaggccctgcgcgtgtcctacaagaagatgaaccccttctgcgtgcccttcgccac
caccaacatgggctccgccatgctggccatggacctgggctggatgggccccaactactcca
tctccaccgcctgcgccacctccaacttctgcatcctgaacgccgccaaccacatcatccgc
ggcgaggccgacatgatgctgtgcggcggctccgacgccgtgatcatccccatcggcctggg
cggcttcgtggcctgccgcgccctgtcccagcgcaactccgaccccaccaaggcctcccgcc
cctgggactccaaccgcgacggcttcgtgatgggcgagggcgccggcgtgctgctgctggag
gagctggagcacgccaagaagcgcggcgccaccatctacgccgagttcctgggcggctcctt
cacctgcgacgcctaccacatgaccgagccccaccccgacggcgccggcgtgatcctgtgca
tcgagaaggccctggcccagtccggcgtgtcccgcgaggacgtgaactacatcaacgcccac
gccacctccacccccgccggcgacatcaaggagtaccaggccctggcccactgcttcggcca
gaactccgagctgcgcgtgaactccaccaagtccatgatcggccacctgctgggcgccgccg
gcggcgtggaggccgtgaccgtgatccaggccatccgcaccggctggatccaccccaacctg
aacctggaggaccccgacgaggccgtggacgccaagttcctggtgggccccaagaaggagcg
cctgaacgtgaaggtgggcctgtccaactccttcggcttcggcggccacaactcctccatcc
tgttcgccccctacaacaccatgtacccctacgacgtgcccgactacgcctgatatcgaggc
agcagcagctcggatagtatcgacacactctggacgctggtcgtgtgatggactgttgccgc
cacacttgctgccttgacctgtgaatatccctgccgcttttatcaaacagcctcagtgtgtt
tgatcttgtgtgtacgcgcttttgcgagttgctagctgcttgtgctatttgcgaataccacc
cccagcatccccttccctcgtttcatatcgcttgcatcccaaccgcaacttatctacgctgt
cctgctatccctcagcgctgctcctgctcctgctcactgcccctcgcacagccttggtttgg
gctccgcctgtattctcctggtactgcaacctgtaaaccagcactgcaatgctgatgcacgg
gaagtagtgggatgggaacacaaatggaaagcttatcgcctgctcaagcgggcgctcaacat
gcagagcgtcagcgagacgggctgtggcgatcgcgagacggacgaggccgcctctgccctgt
ttgaactgagcgtcagcgctggctaaggggagggagactcatccccaggctcgcgccagggc
tctgatcccgtctcgggcggtgatcggcgcgcatgactacgacccaacgacgtacgagactg
atgtcggtcccgacgaggagcgccgcgaggcactcccgggccaccgaccatgtttacaccga
ccgaaagcactcgctcgtatccattccgtgcgcccgcacatgcatcatcttttggtaccgac
ttcggtcttgttttacccctacgacctgccttccaaggtgtgagcaactcgcccggacatga
ccgagggtgatcatccggatccccaggccccagcagcccctgccagaatggctcgcgctttc
cagcctgcaggcccgtctcccaggtcgacgcaacctacatgaccaccccaatctgtcccaga
ccccaaacaccctccttccctgcttctctgtgatcgctgatcagcaacaactagtaacaatg
gccaccgcctccaccttctccgccttcaacgcccgctgcggcgacctgcgccgctccgccgg
ctccggcccccgccgccccgcccgccccctgcccgtgcgcgccgccatcggcaacgagcgca
actcctgcaaggtgatcaacggcaccaaggtgaaggacaccgagggcctgaagggctgctcc
accctgcagggccagtccatgctggacgaccacttcggcctgcacggcctggtgttccgccg
caccttcgccatccgctgctacgaggtgggccccgaccgctccacctccatcatggccgtga
tgaaccacctgcaggaggccgcccgcaaccacgccgagtccctgggcctgctgggcgacggc
ttcggcgagaccctggagatgtccaagcgcgacctgatctgggtggtgcgccgcacccacgt
ggccgtggagcgctaccccgcctggggcgacaccgtggaggtggaggcctgggtgggcgcct
ccggcaacaccggcatgcgccgcgacttcctggtgcgcgactgcaagaccggccacatcctg
acccgctgcacctccgtgtccgtgatgatgaacatgcgcacccgccgcctgtccaagatccc
ccaggaggtgcgcgccgagatcgaccccctgttcatcgagaaggtggccgtgaaggagggcg
agatcaagaagctgcagaagctgaacgactccaccgccgactacatccagggcggctggacc
ccccgctggaacgacctggacgtgaaccagcacgtgaacaacatcatctacgtgggctggat
cttcaagtccgtgcccgactccatctccgagaaccaccacctgtcctccatcaccctggagt
accgccgcgagtgcacccgcggcaacaagctgcagtccctgaccaccgtgtgcggcggctcc
tccgaggccggcatcatctgcgagcacctgctgcagctggaggacggctccgaggtgctgcg
cgcccgcaccgagtggcgccccaagcacaccgactccttccagggcatctccgagcgcttcc
cccagcaggagccccacaaggactacaaggaccacgacggcgactacaaggaccacgacatc
gactacaaggacgacgacgacaagtgactcgaggcagcagcagctcggatagtatcgacaca
ctctggacgctggtcgtgtgatggactgttgccgccacacttgctgccttgacctgtgaata
tccctgccgcttttatcaaacagcctcagtgtgtttgatcttgtgtgtacgcgcttttgcga
gttgctagctgcttgtgctatttgcgaataccacccccagcatccccttccctcgtttcata
tcgcttgcatcccaaccgcaacttatctacgctgtcctgctatccctcagcgctgctcctgc
tcctgctcactgcccctcgcacagccttggtttgggctccgcctgtattctcctggtactgc
aacctgtaaaccagcactgcaatgctgatgcacgggaagtagtgggatgggaacacaaatgg
aaagcttgagctcgtgatgggcgagggcgcggccgtgctggtcatggagtcgctggagcacg
cgcagaagcgcggcgcgaccatcctgggcgagtacctggggggcgccatgacctgcgacgcg
caccacatgacggacccgcaccccgagggcctgggcgtgagcacctgcatccgcctggcgct
cgaggacgccggcgtctcgcccgacgaggtcaactacgtcaacgcgcacgccacctccaccc
tggtgggcgacaaggccgaggtgcgcgcggtcaagtcggtctttggcgacatgaagggcatc
aagatgaacgccaccaagtccatgatcgggcactgcctgggcgccgccggcggcatggaggc
cgtcgccacgctcatggccatccgcaccggctgggtgcaccccaccatcaaccacgacaacc
ccatcgccgaggtcgacggcctggacgtcgtcgccaacgccaaggcccagcacaaaatcaac
gtcgccatctccaactccttcggcttcggcgggcacaactccgtcgtcgcctttgcgccctt
ccgcgagtaggcggagcgagcgcgcttggctgaggagggaggcggggtgcgagccctttggc
tgcgcgcgatactctccccgcacgagcagactccacgcgcctgaatctacttgtcaacgagc
aaccgtgtgttttgtccgtggccattcttattatttctccgactgtggccgtactctgtttg
gctgtgcaagcacc
KASI-1ver2_5′::PmHXT1-2v2-ScarMEL1-PmPGK:CvNR:PmSAD2-2v3-
PmSADtp-CpauKASIVa-CvNR:PmACP1-1p-CpSAD1tp_trmd:CcFATB4-
CvNR::KAS1-1ver2_3′
KASI-1ver2_5′-nucleotides 1-750
PmHXT1-2v2-nucleotides 757-1215
ScarMEL1-nucleotides 1222-2637
PmPGK 3′UTR-nucleotides 2654-3098
CvNR-nucleotides 3105-3506
PmSAD2-2v3-nucleotides 3521-4086
PmSADtp-CpauKASIVa-nucleotides 4093-5695
CvNR-nucleotides 5703-6104
PmACP1-1p-nucleotides 6111-6684
CpSAD1tp_trmd:CcFATB4-nucleotides 6694-7839
CvNR-nucleotides 7846-8247
KAS1-1ver2_3′-nucleotides 8261-9004
SEQ ID NO: 19
D725/pSZ1413 Sequence
Construct D725 is written as SAD2B_5′::CrTUB2-ScSUC2-
CpEF1:PmAMT3-PmFADtp_CwFATB2-CvNR:SAD2B_3′
cgcctggagctggtgcagagcatggggcagtttgcggaggagagggtgctccccgtgctgca
ccccgtggacaagctgtggcagccgcaggacttcctgcccgaccccgagtcgcccgacttcg
aggaccaggtggcggagctgcgcgcgcgcgccaaggacctgcccgacgagtactttgtggtg
ctggtgggcgacatgatcacggaggaggcgctgccgacctacatggccatgctcaacacctt
ggacggtgtgcgcgacgacacgggcgcggctgaccacccgtgggcgcgctggacgcggcagt
gggtggccgaggagaaccggcacggcgacctgctgaacaagtactgttggctgacggggcgc
gtcaacatgcgggccgtggaggtgaccatcaacaacctgatcaagagcggcatgaacccgca
gacggacaacaacccttacttgggcttcgtctacacctccttccaggagcgcgccaccaagt
aggtaccctttcttgcgctatgacacttccagcaaaaggtagggcgggctgcgagacggctt
cccggcgctgcatgcaacaccgatgatgcttcgaccccccgaagctccttcggggctgcatg
ggcgctccgatgccgctccagggcgagcgctgtttaaatagccaggcccccgattgcaaaga
cattatagcgagctaccaaagccatattcaaacacctagatcactaccacttctacacaggc
cactcgagcttgtgatcgcactccgctaagggggcgcctcttcctcttcgtttcagtcacaa
cccgcaaactctagaatatcaatgctgctgcaggccttcctgttcctgctggccggcttcgc
cgccaagatcagcgcctccatgacgaacgagacgtccgaccgccccctggtgcacttcaccc
ccaacaagggctggatgaacgaccccaacggcctgtggtacgacgagaaggacgccaagtgg
cacctgtacttccagtacaacccgaacgacaccgtctgggggacgcccttgttctggggcca
cgccacgtccgacgacctgaccaactgggaggaccagcccatcgccatcgccccgaagcgca
acgactccggcgccttctccggctccatggtggtggactacaacaacacctccggcttcttc
aacgacaccatcgacccgcgccagcgctgcgtggccatctggacctacaacaccccggagtc
cgaggagcagtacatctcctacagcctggacggcggctacaccttcaccgagtaccagaaga
accccgtgctggccgccaactccacccagttccgcgacccgaaggtcttctggtacgagccc
tcccagaagtggatcatgaccgcggccaagtcccaggactacaagatcgagatctactcctc
cgacgacctgaagtcctggaagctggagtccgcgttcgccaacgagggcttcctcggctacc
agtacgagtgccccggcctgatcgaggtccccaccgagcaggaccccagcaagtcctactgg
gtgatgttcatctccatcaaccccggcgccccggccggcggctccttcaaccagtacttcgt
cggcagcttcaacggcacccacttcgaggccttcgacaaccagtcccgcgtggtggacttcg
gcaaggactactacgccctgcagaccttcttcaacaccgacccgacctacgggagcgccctg
ggcatcgcgtgggcctccaactgggagtactccgccttcgtgcccaccaacccctggcgctc
ctccatgtccctcgtgcgcaagttctccctcaacaccgagtaccaggccaacccggagacgg
agctgatcaacctgaaggccgagccgatcctgaacatcagcaacgccggcccctggagccgg
ttcgccaccaacaccacgttgacgaaggccaacagctacaacgtcgacctgtccaacagcac
cggcaccctggagttcgagctggtgtacgccgtcaacaccacccagacgatctccaagtccg
tgttcgcggacctctccctctggttcaagggcctggaggaccccgaggagtacctccgcatg
ggcttcgaggtgtccgcgtcctccttcttcctggaccgcgggaacagcaaggtgaagttcgt
gaaggagaacccctacttcaccaaccgcatgagcgtgaacaaccagcccttcaagagcgaga
acgacctgtcctactacaaggtgtacggcttgctggaccagaacatcctggagctgtacttc
aacgacggcgacgtcgtgtccaccaacacctacttcatgaccaccgggaacgccctgggctc
cgtgaacatgacgacgggggtggacaacctgttctacatcgacaagttccaggtgcgcgagg
tcaagtgacaattgacggagcgtcgtgcgggagggagtgtgccgagcggggagtcccggtct
gtgcgaggcccggcagctgacgctggcgagccgtacgccccgagggtccccctcccctgcac
cctcttccccttccctctgacggccgcgcctgttcttgcatgttcagcgacggatcccgcgt
ctcgaacagagcgcgcagaggaacgctgaaggtctcgcctctgtcgcacctcagcgcggcat
acaccacaataaccacctgacgaatgcgcttggttcttcgtccattagcgaagcgtccggtt
cacacacgtgccacgttggcgaggtggcaggtgacaatgatcggtggagctgatggtcgaaa
cgttcacagcctagggatatcgaattcggccgacaggacgcgcgtcaaaggtgctggtcgtg
tatgccctggccggcaggtcgttgctgctgctggttagtgattccgcaaccctgattttggc
gtcttattttggcgtggcaaacgctggcgcccgcgagccgggccggcggcgatgcggtgccc
cacggctgccggaatccaagggaggcaagagcgcccgggtcagttgaagggctttacgcgca
aggtacagccgctcctgcaaggctgcgtggtggaattggacgtgcaggtcctgctgaagttc
ctccaccgcctcaccagcggacaaagcaccggtgtatcaggtccgtgtcatccactctaaag
aactcgactacgacctactgatggccctagattcttcatcaaaaacgcctgagacacttgcc
caggattgaaactccctgaagggaccaccaggggccctgagttgttccttccccccgtggcg
agctgccagccaggctgtacctgtgatcgaggctggcgggaaaataggcttcgtgtgctcag
gtcatgggaggtgcaggacagctcatgaaacgccaacaatcgcacaattcatgtcaagctaa
tcagctatttcctcttcacgagctgtaattgtcccaaaattctggtctaccgggggtgatcc
ttcgtgtacgggcccttccctcaaccctaggtatgcgcgcatgcggtcgccgcgcaactcgc
gcgagggccgagggtttgggacgggccgtcccgaaatgcagttgcacccggatgcgtggcac
cttttttgcgataatttatgcaatggactgctctgcaaaattctggctctgtcgccaaccct
aggatcagcggcgtaggatttcgtaatcattcgtcctgatggggagctaccgactaccctaa
tatcagcccgactgcctgacgccagcgtccacttttgtgcacacattccattcgtgcccaag
acatttcattgtggtgcgaagcgtccccagttacgctcacctgtttcccgacctccttactg
ttctgtcgacagagcgggcccacaggccggtcgcagccactagtatggctatcaagacgaac
aggcagcctgtggagaagcctccgttcacgatcgggacgctgcgcaaggccatccccgcgca
ctgtttcgagcgctcggcgcttcgtgggcgcgcccccaaggccaacggcagcgccgtgagcc
tgaagtccggcagcctgaacaccctggaggacccccccagcagcccccccccccgcaccttc
ctgaaccagctgcccgactggagccgcctgcgcaccgccatcaccaccgtgttcgtggccgc
cgagaagcagttcacccgcctggaccgcaagagcaagcgccccgacatgctggtggactggt
tcggcagcgagaccatcgtgcaggacggcctggtgttccgcgagcgcttcagcatccgcagc
tacgagatcggcgccgaccgcaccgccagcatcgagaccctgatgaaccacctgcaggacac
cagcctgaaccactgcaagagcgtgggcctgctgaacgacggcttcggccgcacccccgaga
tgtgcacccgcgacctgatctgggtgctgaccaagatgcagatcgtggtgaaccgctacccc
acctggggcgacaccgtggagatcaacagctggttcagccagagcggcaagatcggcatggg
ccgcgagtggctgatcagcgactgcaacaccggcgagatcctggtgcgcgccaccagcgcct
gggccatgatgaaccagaagacccgccgcttcagcaagctgccctgcgaggtgcgccaggag
atcgccccccacttcgtggacgccccccccgtgatcgaggacaacgaccgcaagctgcacaa
gttcgacgtgaagaccggcgacagcatctgcaagggcctgacccccggctggaacgacttcg
acgtgaaccagcacgtgagcaacgtgaagtacatcggctggattctggagagcatgcccacc
gaggtgctggagacccaggagctgtgcagcctgaccctggagtaccgccgcgagtgcggccg
cgagagcgtggtggagagcgtgaccagcatgaaccccagcaaggtgggcgaccgcagccagt
accagcacctgctgcgcctggaggacggcgccgacatcatgaagggccgcaccgagtggcgc
cccaagaacgccggcaccaaccgcgccatcagcacctgattaattaactcgaggcagcagca
gctcggatagtatcgacacactctggacgctggtcgtgtgatggactgttgccgccacactt
gctgccttgacctgtgaatatccctgccgcttttatcaaacagcctcagtgtgtttgatctt
gtgtgtacgcgcttttgcgagttgctagctgcttgtgctatttgcgaataccacccccagca
tccccttccctcgtttcatatcgcttgcatcccaaccgcaacttatctacgctgtcctgcta
tccctcagcgctgctcctgctcctgctcactgcccctcgcacagccttggtttgggctccgc
ctgtattctcctggtactgcaacctgtaaaccagcactgcaatgctgatgcacgggaagtag
tgggatgggaacacaaatggaaagcttgagctccagccacggcaacaccgcgcgccttgcgg
ccgagcacggcgacaagaacctgagcaagatctgcgggctgatcgccagcgacgagggccgg
cacgagatcgcctacacgcgcatcgtggacgagttcttccgcctcgaccccgagggcgccgt
cgccgcctacgccaacatgatgcgcaagcagatcaccatgcccgcgcacctcatggacgaca
tgggccacggcgaggccaacccgggccgcaacctcttcgccgacttctccgcggtcgccgag
aagatcgacgtctacgacgccgaggactactgccgcatcctggagcacctcaacgcgcgctg
gaaggtggacgagcgccaggtcagcggccaggccgccgcggaccaggagtacgtcctgggcc
tgccccagcgcttccggaaactcgccgagaagaccgccgccaagcgcaagcgcgtcgcgcgc
aggcccgtcgccttctcctgga
SAD2B_5′::CrTUB2-ScSUC2-CpEF1:PmAMT3-PmFADtp_CwFATB2-
CvNR:SAD2B_3′
SAD2B_5′-nucleotides 1-497
CrTUB2-nucleotides 504-815
ScSUC2-nucleotides 828-2426
CpEF1_3′UTR-nucleotides 2433-2594
PmA4T3-nucleotides 2818-3882
PmFADtp CwFATB2-nucleotides 3889-5061
CvNR-nucleotides 5076-5483
SAD2B_3′-nucleotides 5490-5974
SEQ ID NO: 20
D1681/pSZ2746 Sequence
Construct D1681 is written as KAS1-1_5′::CrTUB2-NeoR-
CvNR:PmUAPA1-ChFATB2-CpCD181:PmAMT3-PmSADtp-CwKASA1-
CvNR::KAS1-1_3′
ctcaccgcgtgaattgctgtcccaaacgtaagcatcatcgtggctcggtcacgcgatcctgg
atccggggatcctagaccgctggtggagagcgctgccgtcggattggtggcaagtaagattg
cgcaggttggcgaagggagagaccaaaaccggaggctggaagcgggcacaacatcgtattat
tgcgtatagtagagcagtggcagtcgcatttcgaggtccgcaacggatctcgcaagctcgct
acgctcacagtaggagaaaggggaccactgcccctgccagaatggtcgcgaccctctccctc
gccggccccgcctgcaacacgcagtgcgtatccggcaagcgggctgtcgccttcaaccgccc
ccatgttggcgtccgggctcgatcaggtgcgctgaggggggtttggtgtgcccgcgcctctg
ggcccgtgtcggccgtgcggacgtggggccctgggcagtggatcagcagggtttgcgtgcaa
atgcctataccggcgattgaatagcgatgaacgggatacggttgcgctcactccatgcccat
gcgaccccgtttctgtccgccagccgtggtcgcccgggctgcgaagcgggaccccacccagc
gcattgtgatcaccggaatgggcgtggggtaccctttcttgcgctatgacacttccagcaaa
aggtagggcgggctgcgagacggcttcccggcgctgcatgcaacaccgatgatgcttcgacc
ccccgaagctccttcggggctgcatgggcgctccgatgccgctccagggcgagcgctgttta
aatagccaggcccccgattgcaaagacattatagcgagctaccaaagccatattcaaacacc
tagatcactaccacttctacacaggccactcgagcttgtgatcgcactccgctaagggggcg
cctcttcctcttcgtttcagtcacaacccgcaaactctagaatatcaatgatcgagcaggac
ggcctccacgccggctcccccgccgcctgggtggagcgcctgttcggctacgactgggccca
gcagaccatcggctgctccgacgccgccgtgttccgcctgtccgcccagggccgccccgtgc
tgttcgtgaagaccgacctgtccggcgccctgaacgagctgcaggacgaggccgcccgcctg
tcctggctggccaccaccggcgtgccctgcgccgccgtgctggacgtggtgaccgaggccgg
ccgcgactggctgctgctgggcgaggtgcccggccaggacctgctgtcctcccacctggccc
ccgccgagaaggtgtccatcatggccgacgccatgcgccgcctgcacaccctggaccccgcc
acctgccccttcgaccaccaggccaagcaccgcatcgagcgcgcccgcacccgcatggaggc
cggcctggtggaccaggacgacctggacgaggagcaccagggcctggcccccgccgagctgt
tcgcccgcctgaaggcccgcatgcccgacggcgaggacctggtggtgacccacggcgacgcc
tgcctgcccaacatcatggtggagaacggccgcttctccggcttcatcgactgcggccgcct
gggcgtggccgaccgctaccaggacatcgccctggccacccgcgacatcgccgaggagctgg
gcggcgagtgggccgaccgcttcctggtgctgtacggcatcgccgcccccgactcccagcgc
atcgccttctaccgcctgctggacgagttcttctgacaattggcagcagcagctcggatagt
atcgacacactctggacgctggtcgtgtgatggactgttgccgccacacttgctgccttgac
ctgtgaatatccctgccgcttttatcaaacagcctcagtgtgtttgatcttgtgtgtacgcg
cttttgcgagttgctagctgcttgtgctatttgcgaataccacccccagcatccccttccct
cgtttcatatcgcttgcatcccaaccgcaacttatctacgctgtcctgctatccctcagcgc
tgctcctgctcctgctcactgcccctcgcacagccttggtttgggctccgcctgtattctcc
tggtactgcaacctgtaaaccagcactgcaatgctgatgcacgggaagtagtgggatgggaa
cacaaatggaaagctgtatagggataaaagcttatagcgactgctaccccccgaccatgtgc
cgaggcagaaattatatacaagaagcagatcgcaattaggcacatcgctttgcattatccac
acactattcatcgctgctgcggcaaggctgcagagtgtatttttgtggcccaggagctgagt
ccgaagtcgacgcgacgagcggcgcaggatccgacccctagacgagcactgtcattttccaa
gcacgcagctaaatgcgctgagaccgggtctaaatcatccgaaaagtgtcaaaatggccgat
tgggttcgcctaggacaatgcgctgcggattcgctcgagtccgctgccggccaaaaggcggt
ggtacaggaaggcgcacggggccaaccctgcgaagccgggggcccgaacgccgaccgccggc
cttcgatctcgggtgtccccctcgtcaatttcctctctcgggtgcagccacgaaagtcgtga
cgcaggtcacgaaatccggttacgaaaaacgcaggtcttcgcaaaaacgtgagggtttcgcg
tctcgccctagctattcgtatcgccgggtcagacccacgtgcagaaaagcccttgaataacc
cgggaccgtggttaccgcgccgcctgcaccagggggcttatataagcccacaccacacctgt
ctcaccacgcatttctccaactcgcgacttttcggaagaaattgttatccacctagtataga
ctgccacctgcaggaccttgtgtcttgcagtttgtattggtcccggccgtcgagcacgacag
atctgggctagggttggcctggccgctcggcactcccctttagccgcgcgcatccgcgttcc
agaggtgcgattcggtgtgtggagcattgtcatgcgcttgtgggggtcgttccgtgcgcggc
gggtccgccatgggcgccgacctgggccctagggtttgttttcgggccaagcgagcccctct
cacctcgtcgcccccccgcattccctctctcttgcagccactagtatggctatcaagacgaa
caggcagcctgtggagaagcctccgttcacgatcgggacgctgcgcaaggccatccccgcgc
actgtttcgagcgctcggcgcttcgtgggcgcgcccagctgcccgactggagccgcctgctg
accgccatcaccaccgtgttcgtgaagtccaagcgccccgacatgcacgaccgcaagtccaa
gcgccccgacatgctggtggacagcttcggcctggagtccaccgtgcaggacggcctggtgt
tccgccagtccttctccatccgctcctacgagatcggcaccgaccgcaccgccagcatcgag
accctgatgaaccacctgcaggagacctccctgaaccactgcaagagcaccggcatcctgct
ggacggcttcggccgcaccctggagatgtgcaagcgcgacctgatctgggtggtgattaaga
tgcagatcaaggtgaaccgctaccccgcctggggcgacaccgtggagatcaacacccgcttc
agccgcctgggcaagatcggcatgggccgcgactggctgatctccgactgcaacaccggcga
gatcctggtgcgcgccaccagcgcctacgccatgatgaaccagaagacccgccgcctgtcca
agctgccctacgaggtgcaccaggagatcgtgcccctgttcgtggacagccccgtgatcgag
gactccgacctgaaggtgcacaagttcaaggtgaagaccggcgacagcatccagaagggcct
gacccccggctggaacgacctggacgtgaaccagcacgtgtccaacgtgaagtacatcggct
ggatcctggagagcatgcccaccgaggtgctggagacccaggagctgtgctccctggccctg
gagtaccgccgcgagtgcggccgcgactccgtgctggagagcgtgaccgccatggaccccag
caaggtgggcgtgcgctcccagtaccagcacctgctgcgcctggaggacggcaccgccatcg
tgaacggcgccaccgagtggcgccccaagaacgccggcgccaacggcgccatctccaccggc
aagaccagcaacggcaactccgtgtccatggactacaaggaccacgacggcgactacaagga
ccacgacatcgactacaaggacgacgacgacaagtgactcgagagcgtccagcgtgtgggat
gaagggtgcgatggaacggggctgccgccccccctctgggcatctagctctgcaccgcacgc
caggaagcccaagccaggccccgtcacactccctcgctgaagtgttccccccctgccccaca
ctcatccaggtatcaacgccatcatgttctacgtccccgtcatcttcaactccctggggagc
gggcgccgcgcgtcgctgctgaacaccatcatcatcaacgccgtcaactttgttaattaaga
attcggccgacaggacgcgcgtcaaaggtgctggtcgtgtatgccctggccggcaggtcgtt
gctgctgctggttagtgattccgcaaccctgattttggcgtcttattttggcgtggcaaacg
ctggcgcccgcgagccgggccggcggcgatgcggtgccccacggctgccggaatccaaggga
ggcaagagcgcccgggtcagttgaagggctttacgcgcaaggtacagccgctcctgcaaggc
tgcgtggtggaattggacgtgcaggtcctgctgaagttcctccaccgcctcaccagcggaca
aagcaccggtgtatcaggtccgtgtcatccactctaaagaactcgactacgacctactgatg
gccctagattcttcatcaaaaacgcctgagacacttgcccaggattgaaactccctgaaggg
accaccaggggccctgagttgttccttccccccgtggcgagctgccagccaggctgtacctg
tgatcgaggctggcgggaaaataggcttcgtgtgctcaggtcatgggaggtgcaggacagct
catgaaacgccaacaatcgcacaattcatgtcaagctaatcagctatttcctcttcacgagc
tgtaattgtcccaaaattctggtctaccgggggtgatccttcgtgtacgggcccttccctca
accctaggtatgcgcgcatgcggtcgccgcgcaactcgcgcgagggccgagggtttgggacg
ggccgtcccgaaatgcagttgcacccggatgcgtggcaccttttttgcgataatttatgcaa
tggactgctctgcaaaattctggctctgtcgccaaccctaggatcagcggcgtaggatttcg
taatcattcgtcctgatggggagctaccgactaccctaatatcagcccgactgcctgacgcc
agcgtccacttttgtgcacacattccattcgtgcccaagacatttcattgtggtgcgaagcg
tccccagttacgctcacctgtttcccgacctccttactgttctgtcgacagagcgggcccac
aggccggtcgcagcccatatggcttccgcggcattcaccatgtcggcgtgccccgcgatgac
tggcagggcccctggggcacgtcgctccggacggccagtcgccacccgcctgaggtacgtat
tccagtgcctggtggccagctgcatcgacccctgcgaccagtaccgcagcagcgccagcctg
agcttcctgggcgacaacggcttcgccagcctgttcggcagcaagcccttcatgagcaaccg
cggccaccgccgcctgcgccgcgccagccacagcggcgaggccatggccgtggccctgcagc
ccgcccaggaggccggcaccaagaagaagcccgtgatcaagcagcgccgcgtggtggtgacc
ggcatgggcgtggtgacccccctgggccacgagcccgacgtgttctacaacaacctgctgga
cggcgtgagcggcatcagcgagatcgagaccttcgactgcacccagttccccacccgcatcg
ccggcgagatcaagagcttcagcaccgacggctgggtggcccccaagctgagcaagcgcatg
gacaagttcatgctgtacctgctgaccgccggcaagaaggccctggccgacggcggcatcac
cgacgaggtgatgaaggagctggacaagcgcaagtgcggcgtgctgatcggcagcggcatgg
gcggcatgaaggtgttcaacgacgccatcgaggccctgcgcgtgagctacaagaagatgaac
cccttctgcgtgcccttcgccaccaccaacatgggcagcgccatgctggccatggacctggg
ctggatgggccccaactacagcatcagcaccgcctgcgccaccagcaacttctgcatcctga
acgccgccaaccacatcatccgcggcgaggccgacatgatgctgtgcggcggcagcgacgcc
gtgatcatccccatcggcctgggcggcttcgtggcctgccgcgccctgagccagcgcaacag
cgaccccaccaaggccagccgcccctgggacagcaaccgcgacggcttcgtgatgggcgagg
gcgccggcgtgctgctgctggaggagctggagcacgccaagaagcgcggcgccaccatctac
gccgagttcctgggcggcagcttcacctgcgacgcctaccacatgaccgagccccaccccga
gggcgccggcgtgatcctgtgcatcgagaaggccctggcccaggccggcgtgagcaaggagg
acgtgaactacatcaacgcccacgccaccagcaccagcgccggcgacatcaaggagtaccag
gccctggcccgctgcttcggccagaacagcgagctgcgcgtgaacagcaccaagagcatgat
cggccacctgctgggcgccgccggcggcgtggaggccgtgaccgtggtgcaggccatccgca
ccggctggattcaccccaacctgaacctggaggaccccgacaaggccgtggacgccaagctg
ctggtgggccccaagaaggagcgcctgaacgtgaaggtgggcctgagcaacagcttcggctt
cggcggccacaacagcagcatcctgttcgccccctgcaacgtgtgactcgaggcagcagcag
ctcggatagtatcgacacactctggacgctggtcgtgtgatggactgttgccgccacacttg
ctgccttgacctgtgaatatccctgccgcttttatcaaacagcctcagtgtgtttgatcttg
tgtgtacgcgcttttgcgagttgctagctgcttgtgctatttgcgaataccacccccagcat
ccccttccctcgtttcatatcgcttgcatcccaaccgcaacttatctacgctgtcctgctat
ccctcagcgctgctcctgctcctgctcactgcccctcgcacagccttggtttgggctccgcc
tgtattctcctggtactgcaacctgtaaaccagcactgcaatgctgatgcacgggaagtagt
gggatgggaacacaaatggaaagcttgagctccacctgcatccgcctggcgctcgaggacgc
cggcgtctcgcccgacgaggtcaactacgtcaacgcgcacgccacctccaccctggtgggcg
acaaggccgaggtgcgcgcggtcaagtcggtctttggcgacatgaagggcatcaagatgaac
gccaccaagtccatgatcgggcactgcctgggcgccgccggcggcatggaggccgtcgccac
gctcatggccatccgcaccggctgggtgcaccccaccatcaaccacgacaaccccatcgccg
aggtcgacggcctggacgtcgtcgccaacgccaaggcccagcacaaaatcaacgtcgccatc
tccaactccttcggcttcggcgggcacaactccgtcgtcgcctttgcgcccttccgcgagta
ggcggagcgagcgcgcttggctgaggagggaggcggggtgcgagccctttggctgcgcgcga
tactctccccgcacgagcagactccacgcgcctgaatctacttgtcaacgagcaaccgtgtg
ttttgtccgtggccattcttattatttctccgactgtggccgtactctgtttggctgtgcaa
gcacc
KAS1-1_5′::CrTUB2-NeoR-CvNR:PmUAPA1-ChFATB2-CpCD181:PmAMT3-
PmSADtp-CwKASA1-CvNR::KAS1-1_3′
KSI-1_5′-nucleotides 1-646
CrTUB2-nucleotides 654-965
NeoR-nucleotides 978-1772
CvNR-nucleotides 1779-2180
PmUAPA1-nucleotides 2204-3201
ChFATB2-nucleotides 3322-4377
CpCD181-nucleotides 4384-4648
PmA4T3-nucleotides 4655-5719
PmSADtp-CwKASA1-nucleotides 5723-7300
CvNR-nucleotides 7307-7707
KSI-1_3′-nucleotides 7721-8313

1. A method of preparing a triglyceride oil, the triglyceride oil comprising a first population of asymmetric triglyceride molecules and/or a second population of asymmetric triglyceride molecules, the first population comprising triglyceride molecules consisting of a C8:0 fatty acid or a C10:0 fatty acid at the sn-1 position and the sn-2 position, and C14:0, C16:0 or C18:0 at the sn-3 position, the second population comprising triglyceride molecules consisting of a C14:0, C16:0 fatty acid or a C18:0 fatty acid at the sn-1 position and the sn-2 position, and C8:0 or C10:0 fatty acid at the sn-3 position, the method comprising the steps of:
a. obtaining a triglyceride oil isolated from a recombinant microalgal cell, wherein the recombinant microalgal cell comprises an exogenous gene encoding an active sucrose invertase; and
b. hydrogenating the triglyceride oil to produce the asymmetric triglyceride molecules.

2. The method of claim 1, wherein the first population or the second population of triglyceride molecules is enriched by fractionation or preparative liquid chromatography.

3. The method of claim 1, wherein the first population of triglyceride molecules comprises at least 20% of all triglyceride molecules.

4. The method of claim 1, wherein the first population of triglyceride molecules comprises at least 30% of all triglyceride molecules.

5. The method of claim 1, wherein the first population of triglyceride molecules comprises at least 40% of all triglyceride molecules.

6. The method of claim 1, wherein the second population of triglyceride molecules comprises at least 15% of all triglyceride molecules.

7. The method of claim 1, wherein the second population of triglyceride molecules comprises at least 20% of all triglyceride molecules.

8. The method of claim 1, wherein the second population of triglyceride molecules comprises at least 25% of all triglyceride molecules.

9. The method of claim 1, wherein the first and second populations of triglyceride molecules together comprises at least 40% of all triglyceride molecules.

10. The method of claim 1, wherein the first and second populations of triglyceride molecules together comprises at least 45% of all triglyceride molecules.

11. The method of claim 1, wherein together the first and second populations of triglyceride molecules comprises at least 50% of all triglyceride molecules.

12. The method of claim 1, wherein together the first and second populations of triglyceride molecules comprises at least 60% of all triglyceride molecules.

13. The method of claim 1, wherein the triglyceride oil has less than 9 kilocalories per gram.

14. The method of claim 13, wherein the triglyceride oil has 5 to 8 kilocalories per gram.

15. The method of claim 14, wherein the triglyceride oil has 6 to 8 kilocalories per gram.

16. The method of claim 1, wherein the triglyceride oil is a solid at ambient temperature and pressure.

17. The method of claim 1, wherein the triglyceride oil is a structuring fat, laminating fat or a coating fat.

18. The method of claim 1, wherein the melting curve of the asymmetric triglyceride oil has one or more melting points at about 17° C., 31° C., and 37° C.

19. The method of claim 1, wherein the triglyceride oil forms a crystalline polymorph of the β or β′ form.

20. The method of claim 1, wherein the recombinant microalgal cell further comprises one or more exogenous gene encoding a fatty acyl-ACP thioesterase, a ketoacyl-ACP synthase, or a desaturase enzyme.

21. The method of any claim 1, wherein the recombinant microalgal cell further comprises one or more exogenous gene that disrupts the expression of an endogenous gene encoding a fatty acyl-ACP thioesterase, a ketoacyl-ACP synthase, or a desaturase enzyme.

22. A triglyceride oil produced by the method of claim 1.
